(19) United States
(12) Patent Application Publication
(10) Pub. No.: US 2025/0035631 A1
(43) Pub. Date: Jan. 30, 2025

(54) METHOD OF DETECTING ADENOMA

(71) Applicant: Vision Tech Bio Pty Ltd, Parkville, Victoria (AU)

(72) Inventors: Trevor LOCKETT, Parkville, Victoria (AU); Michael BUCKLEY, New South Wales (AU); Craig LEWIS, Parkville, Victoria (AU); Christian DAISH, Parkville, Victoria (AU); Louise FORMBY-MILLER, Parkville, Victoria (AU); Leah COSGROVE, Australian Capital Territory (AU); Kim Yoke Ching FUNG, Australian Capital Territory (AU); Ilka PRIEBE, Australian Capital Territory (AU)

(73) Assignees: Vision Tech Bio Pty Ltd, Parkville, Victoria (AU); Commonwealth Scientific and Industrial Research Organisation, Australian Capital Territory (AU)

(21) Appl. No.: 18/682,862

(22) PCT Filed: Aug. 11, 2022
(86) PCT No.: PCT/AU2022/050882

§ 371 Date: Feb. 9, 2024

(30) Foreign Application Priority Data
Aug. 11, 2021 (AU) 2021902501

(51) Int. Cl. G01N 33/574

(52) U.S. Cl. CPC G01N 33/57419; G01N 2333/4703; G01N 2333/4745; G01N 2333/495; G01N 2333/5421; G01N 2333/5437; G01N 2333/8146

(57) ABSTRACT

The disclose relates to adenomas of the colon. More particularly, the present disclosure relates to biomarkers which may be used for the detection of advanced colorectal pre-cancerous adenomas (APA) The detection and measurement of these biomarkers in a biological sample may be used to inform the clinician as to whether further invasive procedures such as polypectomy are required.

This application claims priority to AU 2021902501 filed 11 Aug. 2021, the entire contents of which are herein incorporated by reference.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

### REFERENCE TO SEQUENCE LISTING

The entire content of the electronic submission of the sequence listing is incorporated by reference in its entirety for all purposes.

### FIELD OF THE DISCLOSURE

The disclose relates to adenomas of the colon. More particularly, the present disclosure relates to biomarkers which are associated with a higher risk of developing colorectal cancer. The detection and measurement of these biomarkers in a biological sample may be used to inform the clinician as to whether further invasive procedures such as polypectomy are required.

### BACKGROUND

Colorectal cancers most often begin within otherwise benign outgrowths of the colonic mucosa called adenomas which can develop into a malignant tumour over ten to twenty years. If adenomas are found at an early stage, surgical treatment is effective and complete recovery is possible before any chance of malignant transformation.

An adenoma is a benign tumour of epithelial tissue with glandular origin. While most adenomas are benign, over time they can transform to become malignant at which point they are called adenocarcinomas. Although most adenomas do not transform, even while benign, they have the potential to cause serious health complications by compressing other structures or by producing large amounts of hormones in an unregulated, non-feedback-dependent manner (causing paraneoplastic syndromes).

In the case of benign colorectal adenomas, low invasive endoscopic resection can be performed. Even in the case of a malignant tumour, if it is at an early stage, an endoscopic resection can be performed. Furthermore, even in the case of advanced cancer, surgical treatments are often effective. Because of the slow development process of colorectal cancer, there is an opportunity for prevention and early intervention. Accordingly, it is possible to reduce the morbidity rate and the mortality rate of colorectal adenoma and even reduce the incidence of colorectal cancer through early stage detection and resection of adenomas.

The currently performed adenoma or cancer detection methods such as the fecal occult blood test (FOBT), double contrast barium enema, sigmoidoscopy and total colonoscopy have various issues.

The FOBT detects blood contained in the faeces from a bleeding adenoma or cancer. However, many cases of adenoma or early-stage tumour may result in false negatives, and thus the sensitivity cannot be said to be sufficient. Moreover, cases of bleeding which occurs not from an adenoma or tumour but from a non-neoplastic intestinal tract lesion or injury (such as haemorrhoid) may result in false positives, and thus the specificity cannot be said to be high.

The barium enema is an X-ray photographic method in which barium and air are injected from the anus after a thorough laxative pre-treatment. This test can clarify the accurate position and size of cancer, the degree of narrowness of the intestine, and the like. Therefore, it is possible to detect a large-shaped advanced cancer. However, the shortcoming is that it is difficult to detect a small-shaped early-stage cancer or a flattened cancer and detection of neoplasia still requires colonoscopy to confirm diagnosis and to inform the most appropriate treatment choice.

Sigmoidoscopy and total colonoscopy are videoscopic methods in which the inside of the intestine is observed after a thorough laxative pre-treatment. The laxative pre-treatment in these methods requires the administration of two to three litres of laxative, which imposes an unpleasant burden on the subject. Furthermore, tearing or perforation may occur during the method. Accordingly, such methods are not ideal in screening for adenoma.

Accordingly, these methods have certain disadvantages for screening for adenomas.

Furthermore, reliance on bodily fluid samples such as excrement samples are not easily obtained and do not give reliable results. The use of faeces as a specimen has several problems. First, various types of substances can be present in faeces including substances derived from cancer cells or pathogenic bacteria. Furthermore, collection methods, storage handling of the faeces samples may also affect the accuracy of the screening test on that sample.

Therefore, there is a need for a low invasive test method which is able to screen for early-stage detection of colorectal cancer, particularly at the level of advanced adenoma detection. Such methods would thus identify patients who require further investigation by colonoscopy while minimising the number of unnecessary colonoscopies.

### SUMMARY OF THE DISCLOSURE

The inventors investigated blood biomarkers associated with advanced adenoma detection, more particularly advanced pre-cancerous adenomas (APA) in subjects. The present disclosure is based on the finding that certain blood biomarkers are useful for detecting APA in a subject. Accordingly, methods are described here which provide for the identification of subjects having, or at greater risk of having, APA and/or subjects having multiple small adenomas or polyps. The methods of the present disclosure also provide for detection of sessile serrated adenomas of any size within a subject.

A panel of biomarkers including brain derive neurotrophic factor (BDNF), insulin-like growth factor binding protein 2 (IGFBP2), dickkoph-related protein 3 (DKK-3), tumour pyruvate kinase isozyme M2 (PK-M2, also referred to herein as M2PK), Mac-2 binding protein (Mac2BP), transforming growth factor beta 1 (TGFβ1), tissue inhibitor matrix metalloproteinase 1 (TIMP1), interleukin 8 (IL-8), interleukin 13 (IL-13) and endothelial cell adhesion molecule (EpCAM) were investigated to find biomarker combinations that would identify subjects at higher risk of developing early stage colorectal cancer, for example, subjects at greater risk of presenting with adenoma and/or polyps and therefore requiring further investigation by a more invasive method such as sigmoidoscopy or colonoscopy. The methods of the present disclosure can be used to identify patients at greater risk for adenoma and/or polyp detection in the colon and rectum. The methods of the present disclosure can alternatively or additionally be used for detecting multiple adenomas and/or polyps in a subject. Alternatively or additionally, the present methods can be used for detecting the presence and/or level of protein biomarkers in a subject suspected of having advanced pre-cancerous adenoma (APA), including advanced colorectal adenoma.

In a first aspect, there is provided a method for the detection of colorectal pre-cancerous adenomas (APA) in a subject, the method comprising:
determining a measurement for a panel of biomarkers in a biological sample obtained from the subject, the panel comprising at least IGFBP2 and one or more further biomarkers selected from the group consisting of DKK-3, tumour M2PK, Mac2BP, TGFβ1, TIMP1, IL-8, IL-13 and EpCAM;
wherein the measurement comprises measuring a level of each of the biomarkers in the panel.

In one example, there is provided a method for the detection of colorectal pre-cancerous adenomas (APA) in a subject, the method comprising:
determining a measurement for a panel of biomarkers in a biological sample obtained from the subject, the panel comprising:
   at least IGFBP2 and one or more further biomarkers selected from the group consisting of DKK-3, tumour M2PK, Mac2BP, TGFβ1, TIMP1, IL-8, IL-13 and EpCAM; and
   optionally brain-derived neurotrophic factor (BDNF);
wherein the measurement comprises measuring a level of each of the biomarkers in the panel.

In one example, the panel of biomarkers comprises BDNF and IGFBP2.

In one example, the adenoma is a colorectal adenoma.

In another example, the adenoma is an advanced colorectal adenoma.

In another example, the biological sample is blood, plasma or serum. In another example, the biological sample is another bodily fluid such as saliva or urine.

In one example according to the first aspect and any further aspect described herein, determining a measurement comprises detecting biomarkers in the biological sample by contacting the sample with detectable binding agents that specifically bind to the biomarkers. In a further example, the method comprises detecting specific binding between the specific binding agents and the biomarkers using a detection assay. In a further example, determining a measurement comprises measuring the concentration of biomarker in the biological sample. In a further example, determining a measurement comprises performing a statistical analysis.

In one example, the method comprises detecting IGFBP2 and two further biomarkers selected from the group consisting of DKK-3, tumour M2PK, Mac2BP, TGFβ1, TIMP1, IL-8, IL-13 and EpCAM.

In one example, the method comprises detecting IGFBP2 and three further biomarkers selected from the group consisting of DKK-3, tumour M2PK, Mac2BP, TGFβ1, TIMP1, IL-8, IL-13 and EpCAM.

In one example, the method comprises detecting IGFBP2 and at least four biomarkers selected from the group consisting of DKK-3, tumour M2PK, Mac2BP, TGFβ1, TIMP1, IL-8, IL-13 and EpCAM.

In one example, the method comprises detecting IGFBP2 and at least five biomarkers selected from the group consisting of DKK-3, tumour M2PK, Mac2BP, TGFβ1, TIMP1, IL-8, IL-13 and EpCAM.

In one example, the method comprises detecting IGFBP2 and at least six biomarkers selected from the group consisting of DKK-3, tumour M2PK, Mac2BP, TGFβ1, TIMP1, IL-8, IL-13 and EpCAM.

In one example, the method comprises detecting IGFBP2 and at least seven biomarkers selected from the group consisting of DKK-3, tumour M2PK, Mac2BP, TGFβ1, TIMP1, IL-8, IL-13 and EpCAM.

In one example, the method comprises detecting biomarkers IGFBP2, DKK-3, tumour M2PK, Mac2BP, TGFβ1, TIMP1, IL-8, IL-13 and EpCAM.

In one example, the method comprises detecting IGFBP2, BDNF and two further biomarkers selected from the group consisting of DKK-3, tumour M2PK, Mac2BP, TGFβ1, TIMP1, IL-8, IL-13 and EpCAM.

In one example, the method comprises detecting IGFBP2, BDNF and three further biomarkers selected from the group consisting of DKK-3, tumour M2PK, Mac2BP, TGFβ1, TIMP1, IL-8, IL-13 and EpCAM.

In one example, the method comprises detecting IGFBP2, BDNF and at least four biomarkers selected from the group consisting of DKK-3, tumour M2PK, Mac2BP, TGFβ1, TIMP1, IL-8, IL-13 and EpCAM.

In one example, the method comprises detecting IGFBP2, BDNF and at least five biomarkers selected from the group consisting of DKK-3, tumour M2PK, Mac2BP, TGFβ1, TIMP1, IL-8, IL-13 and EpCAM.

In one example, the method comprises detecting IGFBP2, BDNF and at least six biomarkers selected from the group consisting of DKK-3, tumour M2PK, Mac2BP, TGFβ1, TIMP1, IL-8, IL-13 and EpCAM.

In one example, the method comprises detecting IGFBP2, BDNF and at least seven biomarkers selected from the group consisting of DKK-3, tumour M2PK, Mac2BP, TGFβ1, TIMP1, IL-8, IL-13 and EpCAM.

In one example, the method comprises detecting biomarkers IGFBP2, DKK-3, tumour M2PK, TIMP1, and BDNF.

In certain examples, two biomarkers are detected, three biomarkers are detected, four biomarkers are detected, five biomarkers are detected, six biomarkers are detected, seven biomarkers are detected, eight biomarkers are detected or nine biomarkers are detected.

In one example, the biomarkers are protein biomarkers.

The biological sample may be selected from the group consisting of whole blood, plasma or serum.

In one example, the method comprises detecting IGFBP2 and one further biomarker or at least one further biomarker selected from the group consisting of Mac2BP, TIMP1, TGFβ1, EpCAM and IL-13.

In one example, the three biomarker panels are selected from:
(i) IGFBP2, Mac2BP, TIMP1; and
(ii) IGFBP2, Mac2BP, TGFβ1.

In one example, the four biomarker panels are selected from:
(i) IGFBP2, Mac2BP, TGFβ1, IL-13;
(ii) IGFBP2, Mac2BP, TIMP1, IL-13; and
(iii) IGFBP2, Mac2BP, TIMP1, EpCAM.

In one example, the five biomarker panels are selected from:
(i) IGFBP2, Mac2BP, TGFβ1, TIMP1, EpCAM;
(ii) IGFBP2, M2PK, IL-13, TIMP1, EpCAM;
(iii) IGFBP2, Mac2BP, TGFβ1, M2PK, EpCAM;
(iv) IGFBP2, Mac2BP, IL-13, TIMP1, EpCAM;
(v) IGFBP2, Mac2BP, TGFβ1, TIMP1, IL-13;
(vi) IGFBP2, M2PK, IL-13, TIMP1, IL-8; and
(vii) IGFBP2, Mac2BP, IL-13, TIMP1, DKK3.

In one example, the six biomarker panels comprise IGFBP2, TIMP1, IL-13 and Mac2BP and a further biomarker selected from DKK3 or EpCAM.

In one example, the six biomarker panels are selected from:
(i) IGFBP2, Mac2BP, TGFβ1, TIMP1, DKK3, IL-13;
(ii) IGFBP2, Mac2BP, M2PK, TIMP1, EpCAM, IL-13; and
(iii) IGFBP2, Mac2BP, M2PK, TIMP1, DKK3; IL-13.

In one example, the biomarker panel further comprises BDNF.

In one example, the biomarker panels comprise IGFBP2 and TIMP1 and a further one or more biomarkers selected from the group consisting of DKK3, BDNF, M2PK, Mac2BP, IL-13 or EpCAM.

In one example, the biomarker panels comprise IGFBP2, TIMP1 and DKK3 and a further one or more biomarkers selected from the group consisting of M2PK, BDNF, Mac2BP, IL-13 and EpCAM.

In one example, the biomarker panel comprises or consists of IGFBP2, TIMP1, DKK3 and M2PK. In one example, the biomarker panel comprises or consists of IGFBP2, TIMP1, DKK3, M2PK and BDNF.

In some examples, the methods of the disclosure also contemplate the inclusion of the subject's age as a biomarker in a biomarker panel described herein.

In a second aspect, there is provided a method for the detection of pre-cancerous colorectal adenomas in a subject, the method comprising:
determining a measurement for a panel of biomarkers in a biological sample obtained from the subject, the panel comprising at least IGFBP2 and the subject's age as a biomarker and one or more further biomarkers selected from the group consisting of DKK-3, tumour M2PK, Mac2BP, TGFβ1, TIMP1, IL-8, IL-13 and EpCAM;
wherein the measurement comprises measuring a level of each of the biomarkers in the panel.

In one example, there is provided a method for the detection of pre-cancerous colorectal adenomas in a subject, the method comprising:
determining a measurement for a panel of biomarkers in a biological sample obtained from the subject, the panel comprising:
   at least IGFBP2 and the subject's age as a biomarker and one or more further biomarkers selected from the group consisting of DKK-3, tumour M2PK, Mac2BP, TGFβ1, TIMP1, IL-8, IL-13 and EpCAM; and
   optionally BDNF;
wherein the measurement comprises measuring a level of each of the biomarkers in the panel.

In one example, the panel of biomarkers comprises BDNF and IGFBP2.

In one example, according to the second aspect, determining a measurement comprises detecting biomarkers in the biological sample by contacting the sample with detectable binding agents that specifically bind to the biomarkers. In a further example, the method comprises detecting specific binding between the specific binding agents and the biomarkers using a detection assay. In a further example, determining a measurement comprises measuring the concentration of biomarker in the biological sample. In a further example, determining a measurement comprises performing a statistical analysis.

In certain examples, two biomarkers are detected, three biomarkers are detected, four biomarkers are detected, five biomarkers are detected, six biomarkers are detected, seven biomarkers are detected, eight biomarkers are detected or nine biomarkers are detected.

In one example according to the second aspect, the biomarker panels are selected from:
(i) IGFBP2 and Mac2BP;
(ii) IGFBP2 and TGFβ1;
(iii) IGFBP2 and TIMP1;
(iv) IGFBP2 and EpCAM;
(v) IGFBP2 and DKK-3; and
(vi) IGFBP2 and M2PK.

In one example according to the second aspect, the biomarker panels are selected from:
(i) IGFBP2, Mac2BP and TIMP1;
(ii) IGFBP2, Mac2BP and TGFβ1;
(iii) IGFBP2, Mac2BP and DKK3;
(iv) IGFBP2, TGFβ1 and TIMP1; and
(v) IGFBP2, TGFβ1 and EpCAM.

In one particular example, the biomarkers are IGFBP2, Mac2BP and TGFβ1 and the subject's age.

In one example according to the second aspect, the biomarker panels are selected from:
(i) IGFBP2, Mac2BP, TGFβ1, DKK3;
(ii) IGFBP2, Mac2BP, TGFβ1, TIMP1;
(iii) IGFBP2, Mac2BP, EpCAM, TIMP1;
(iv) IGFBP2, Mac2BP, IL-13, TIMP1;
(v) IGFBP2, Mac2BP, TGFβ1, IL-13; and
(vi) IGFBP2, EpCAM, TGFβ1, DKK3.

In one example according to the second aspect, the biomarker panels are selected from:
(i) IGFBP2, Mac2BP, TGFβ1, TIMP1, EpCAM;
(ii) IGFBP2, Mac2BP, TGFβ1, TIMP1, M2PK;
(iii) IGFBP2, Mac2BP, TGFβ1, DKK3, IL-13;
(iv) IGFBP2, Mac2BP, TGFβ1, TIMP1, IL-13; and
(v) IGFBP2, Mac2BP, TGFβ1, TIMP1, DKK3

In one example according to the second aspect, the biomarker panels are selected from:
(i) IGFBP2, Mac2BP, TGFβ1, TIMP1, IL-8, EpCAM; and
(ii) IGFBP2, Mac2BP, TGFβ1, TIMP1, DKK3, IL-13.

In one example, the biomarker panel further comprises BDNF.

In one example, the biomarker panel comprises IGFBP2 and TIMP1 and a further one or more biomarkers selected from the group consisting of DKK3, BDNF, M2PK, Mac2BP, IL-13 or EpCAM.

In one example, the biomarker panel comprises IGFBP2, TIMP1 and DKK3 and a further one or more biomarkers selected from the group consisting of M2PK, BDNF, Mac2BP, IL-13 and EpCAM.

In one example according to the second aspect, the biomarker panel comprises or consists of IGFBP2, TIMP1, DKK3 and M2PK and the subject's age as a biomarker. In one example according to the second aspect, the biomarkers comprise or consist of IGFBP2, TIMP1, DKK3, M2PK and BDNF and the subject's age as a biomarker. In some examples, the methods of the disclosure also contemplate the inclusion of the subject's gender as a biomarker in a biomarker panel described herein.

In a third aspect, there is provided a method for the detection of pre-cancerous colorectal adenomas in a subject, the method comprising:
determining a measurement for a panel of biomarkers in a biological sample obtained from the subject, the panel comprising at least IGFBP2 and the subject's gender as a biomarker and one or more further biomarkers selected from the group consisting of DKK-3, tumour M2PK, Mac2BP, TGFβ1, TIMP1, IL-8, IL-13 and EpCAM;
wherein the measurement comprises measuring a level of each of the biomarkers in the panel.

In one example, there is provided a method for the detection of pre-cancerous colorectal adenomas in a subject, the method comprising:
determining a measurement for a panel of biomarkers in a biological sample obtained from the subject, the panel comprising:
at least IGFBP2 and the subject's gender as a biomarker and one or more further biomarkers selected from the group consisting of DKK-3, tumour M2PK, Mac2BP, TGFβ1, TIMP1, IL-8, IL-13 and EpCAM; and
optionally BDNF;
wherein the measurement comprises measuring a level of each of the biomarkers in the panel,
In one example, the panel of biomarkers comprises BDNF and IGFBP2.

In some examples, gender is factored into the method by separating the samples from males and females and analysing them separately. Alternatively, gender is factored into the algorithm by assigning an arbitrary value for females and a different arbitrary value for males. In one example, the subject's gender is factored into the algorithm by assigning an arbitrary value for males and females (for example, 1.1 for females and 1.0 for males or 1.0 for females and 0 for males). In one example, the subject's gender is factored into the algorithm by assigning an arbitrary value of 1.0 for females and 0 for males.

In one example, according to the third aspect, determining a measurement comprises detecting biomarkers in the biological sample by contacting the sample with detectable binding agents that specifically bind to the biomarkers. In a further example, the method comprises detecting specific binding between the specific binding agents and the biomarkers using a detection assay. In a further example, determining a measurement comprises measuring the concentration of biomarker in the biological sample. In a further example, determining a measurement comprises performing a statistical analysis.

In certain examples, two biomarkers are detected, three biomarkers are detected, four biomarkers are detected, five biomarkers are detected, six biomarkers are detected, seven biomarkers are detected, eight biomarkers are detected or nine biomarkers are detected.

In one example according to the third aspect, the biomarker panels are selected from:
(i) IGFBP2 and TIMP1; and
(ii) IGFBP2 and IL-13.

In one example according to the third aspect, the biomarker panels are selected from:
(i) IGFBP2, Mac2BP, TIMP1;
(ii) IGFBP2, Mac2BP, IL-13;
(iii) IGFBP2, Mac2BP, TGFβ1;
(iv) IGFBP2, IL-8, IL-13;
(v) IGFBP2, DKK-3, IL-13; and
(vi) IGFBP2, IL-13, EpCAM.

In one example according to the third aspect, the biomarker panels are selected from:
(i) IGFBP2, Mac2BP, IL-8, IL-13;
(ii) IGFBP2, Mac2BP, M2PK, TIMP1;
(iii) IGFBP2, Mac2BP, TGFβ1, EpCAM;
(iv) IGFBP2, M2PK, TIMP1, IL-13;
(v) IGFBP2, Mac2BP, M2PK, IL-13;
(vi) IGFBP2, Mac2BP, TGFβ1, TIMP1;
(vii) IGFBP2, IL-8, IL-13, EpCAM;
(viii) IGFBP2, IL-8, IL-13, TIMP1;
(ix) IGFBP2, IL-8, IL-13, DKK3;
(x) IGFBP2, Mac2BP, IL-13, TIMP1;
(xi) IGFBP2, Mac2BP, TGFβ1, IL-13;
(xii) IGFBP2, Mac2BP, DKK3, TIMP1;
(xiii) IGFBP2, EpCAM, IL-13, TIMP1;
(xiv) IGFBP2, Mac2BP, IL-13, DKK3;
(xv) IGFBP2, EpCAM, IL-13, DKK3; and
(xvi) IGFBP2, TGFβ1, IL-13, IL-8

In one example according to the third aspect, the biomarker panels are selected from:
(i) IGFBP2, Mac2BP, IL-8, IL-13, EpCAM;
(ii) IGFBP2, TGFβ1, IL-8, IL-13, TIMP1;
(iii) IGFBP2, M2PK, EpCAM, IL-13, TIMP1;
(iv) IGFBP2, Mac2BP, IL-8, IL-13, DKK3;
(v) IGFBP2, Mac2BP, M2PK, IL-13, TGFβ1;
(vi) IGFBP2, DKK3, IL-8, IL-13, EpCAM;
(vii) IGFBP2, M2PK, IL-8, IL-13, TIMP1;
(viii) IGFBP2, Mac2BP, IL-8, TGFβ1, TIMP1;
(ix) IGFBP2, Mac2BP, M2PK, IL-13, EpCAM;
(x) IGFBP2, M2PK, TGFβ1, IL-13, TIMP1;
(xi) IGFBP2, Mac2BP, DKK3, IL-13, TIMP1;
(xii) IGFBP2, Mac2BP; DKK3, IL-8, TIMP1;
(xiii) IGFBP2, Mac2BP, M2PK, TGFβ1, TIMP1;
(xiv) IGFBP2, EpCAM, IL-8, IL-13, TIMP1;
(xv) IGFBP2, M2PK, IL-8, IL-13, EpCAM;
(xvi) IGFBP2, Mac2BP, M2PK, DKK3, IL-13;
(xvii) IGFBP2, Mac2BP, TIMP1, IL-13, EpCAM; and
(xviii) IGFBP2, DKK3, IL-8, IL-13, TIMP1.

In one example according to the third aspect, the biomarker panels are selected from:
(i) IGFBP2, Mac2BP, M2PK, DKK3, IL-8, IL-13;
(ii) IGFBP2, Mac2BP, TIMP1, EpCAM, IL-8, IL-13;
(iii) IGFBP2, Mac2BP, TIMP1, DKK3, IL-8, IL-13;
(iv) IGFBP2, Mac2BP, Dkk3, EpCAM, IL8, IL13;
(v) IGFBP2, TGFβ1, TIMP1, EpCAM, IL-8, IL-13;
(vi) IGFBP2, M2PK, TIMP1, TGFβ1, EpCAM, IL-13; and
(vii) IGFBP2, Mac2BP; M2PK, TIMP1, IL-8, DKK3.

In one example according to the third aspect, the biomarker panel is IGFBP2, Mac2BP, DKK3, IL-8, EpCAM, TIMP1, and IL-13.

In one example, the biomarker panel further comprises BDNF.

In one example, the biomarker panel comprises IGFBP2 and TIMP1 and a further one or more biomarkers selected from the group consisting of DKK3, BDNF, M2PK, Mac2BP, IL-13 or EpCAM.

In one example, the biomarker panel comprises IGFBP2, TIMP1 and DKK3 and a further one or more biomarkers selected from the group consisting of M2PK, BDNF, Mac2BP, IL-13 and EpCAM.

In one example according to the third aspect, the biomarkers comprise or consist of IGFBP2, TIMP1, DKK3 and M2PK and the subject's gender as a biomarker. In one example according to the third aspect, the biomarkers comprise or consist of IGFBP2, TIMP1, DKK3, M2PK and BDNF and the subject's gender as a biomarker.

In some examples, the methods of the disclosure also contemplate the inclusion of the subject's age as a biomarker in a biomarker panel described herein.

The methods of the disclosure involve detecting the presence of biomarkers in a biological sample from a subject, preferably a human subject, by determining a measurement for each biomarker in the sample. In some examples this may comprise measuring expression of a given biomarker. In other examples this may comprise measuring the concentration of a given biomarker in the sample.

In some examples, the level of at least one biomarker in the panel of biomarkers is increased or decreased relative to a level of the same biomarker in a reference panel.

More particularly, the measurement of a biomarker is relative to a reference concentration for that biomarker determined in known cases of APA and/or control samples by an algorithm trained on the case and control samples.

In some examples, the methods of the disclosure comprise:
(i) performing a measurement of the concentration of each biomarker in a biomarker panel described herein;
(ii) inputting the values from (i) into an algorithm that has been determined to maximise the differentiation between cases and controls based on known case and control data;
(iii) obtaining an APA likelihood score; and optionally
(iv) comparing the value obtained in step (iii) with a threshold value.

In one example, the threshold value is derived from known case and control samples that give the highest sensitivity for differentiation between cases and controls at a given specificity. In one example, the specificity is 86.4% or greater. In another example, the specificity is 90%, a yet a further example the specificity is 95%.

In certain examples, the biomarker reference panel is the corresponding biomarkers measured in control and case subjects.

In some examples, the biomarkers are protein biomarkers. In one example, the biomarkers are polynucleotide biomarkers.

In some examples, the methods of the disclosure comprise contacting the biological sample with antibodies that specifically bind to the biomarker proteins. Preferably, there is at least one antibody that binds individually to each biomarker sought to be detected in the biological sample. Preferably, the antibodies specifically bind to a given biomarker. In some examples, more than one antibody may bind to a single biomarker, for example in a “sandwich” format.

In some examples, the measuring format is an immunoassay. In another example, the immunoassay is an ELISA, typically there would be a capture antibody bound to the surface of the ELISA plate and a detection antibody to detect binding of the biomarker to the capture antibody. In one example, the detection antibody may be detectably labelled. In one example the capture antibody may be the same antibody or a different antibody to the detection antibody. Methods of labelling antibodies are known in the art.

In some examples, the expression or concentration of a biomarker, e.g., IGFBP2, will be higher in an APA subject compared to a reference value determined from controls, however for certain biomarkers, e.g., DKK3, expression or concentration of that biomarker is decreased relative to a reference value from controls. In another example, the detection of a given biomarker comprises performing mass spectrometry on the sample.

In a fourth aspect, the disclosure provides a method of identifying a subject with APA, the method comprising:
(i) contacting a biological sample obtained from the subject with compounds that specifically and individually bind to a panel of biomarkers as set forth herein;
(ii) determining the expression or concentration of each biomarker in the sample to obtain a value for each biomarker;
(iii) inputting the values obtained in step (ii) into a logistic regression algorithm;
(iv) comparing the values obtained in step (iii) to a value obtained from the concentration of the same biomarkers in a corresponding biomarker reference panel of case and control samples; and
(v) obtaining a disease likelihood score.

In some examples, the score is a binary score of positive or negative.

In one example, the method comprises inputting a mathematical transformation of the value obtained in step (ii). In another example, the mathematical transformation is the logarithm of that value.

In some examples, the logistic regression algorithm is as defined herein.

In some examples, the sample is whole blood, plasma or serum. In some examples, the method is performed by ELISA. In some examples, the biomarkers are protein biomarkers. In some examples, step (i) is performed by contacting individual biomarkers with an antibody that specifically binds to that biomarker. In some examples, the method comprises determining the expression and/or concentration of each biomarker relative to a defined threshold value for that biomarker determined from case and control samples. Suitably, case samples are those obtained or derived from subjects with colonoscopically-confirmed colorectal cancer, or preferably subjects with colonoscopically-confirmed advanced neoplasia, such as colorectal cancer and advanced adenoma, or more preferably, subjects with colonoscopically-confirmed advanced adenoma. Suitably, control samples are those obtained or derived from subjects with colorectal lesions other than colorectal cancer and advanced adenoma and/or subjects with substantially no colorectal lesions. The case sample may or may not have colorectal cancer (CRC). In one example, the case sample does not have CRC.

Methods of performing model building and statistical analysis will be known to persons skilled in the art. In some examples, linear or non-linear regression is performed. The methods may also utilise a Baeysian probability algorithm.

In some examples, the disease score is obtained by processing the value obtained in step (ii) via multivariate analysis (e.g., regression analysis). In some examples, the disease score is used to identify APA subjects. In some examples, the subject will be referred onto further testing, for example sigmoidoscopy or colonoscopy; or surgery.

In one example, the method according to this aspect comprises obtaining a disease score for a biomarker combination set forth herein. In another example, the method according to this aspect comprises obtaining a disease score for a biomarker combination set forth in any one of Tables 6 to 28. In another example, the method according to this aspect comprises obtaining a disease score for a biomarker combination set forth in any one of Tables 6 to 28 and 31 to 33.

In one example, the methods herein identify an APA subject with a sensitivity of greater than or equal to 30% at a specificity of 86.4%. In one example, the method identifies an APA subject with a sensitivity of greater than or equal to 32% at a specificity of 86.4%. In one example, the method identifies an APA subject with a sensitivity of greater than or equal to 34% at a specificity of 86.4%. In another example, the methods herein identify an APA subject with a sensitivity of greater than or equal to 30% at the specificity of 90% or 95%.

In one example, the biomarkers are protein or peptide biomarkers. In one example, the biomarkers are cell surface expressed or secreted. In another example, the biomarkers are polynucleotide biomarkers. In one example, the method comprises detecting the biomarker polypeptides by microarray. In another example, the method comprises detecting the biomarkers by ELISA. In another example, concentration of the biomarkers is determined by ELISA.

Methods of analysing biomarkers present in a biological sample will be familiar to persons skilled in the art. In one example, the method comprises detecting and measuring the expression of the biomarker polypeptides by mass spectrometry. In other examples, the method comprises detecting and measuring the expression of the biomarker polypeptides by electrochemiluminescence. In further examples, the method comprises detecting and measuring the expression of the biomarker polypeptides by fluorescence resonance energy transfer (FRET) or proximity extension assay (PEA).

In one example, the methods of the disclosure comprise contacting the biological sample with at least one antibody that binds to a biomarker. Preferably, there is at least one antibody that binds individually to each biomarker sought to be detected in the biological sample. Preferably, the antibodies specifically bind to a given biomarker. In some examples, more than one antibody may bind to a single biomarker. For example, the detection and measuring format is an immunoassay. In another example, the immunoassay is an ELISA, e.g. sandwich ELISA. Typically, a “capture” antibody will be bound to a solid support e.g., the surface of the ELISA plate. The biological sample is then passed over the bound antibodies so as to allow for biomarkers present in the sample to contact the capture antibodies. Binding is confirmed using a “detection” antibody to detect binding of the biomarker to the capture antibody. In one example, the detection antibody may be labelled. In one example the capture antibody may be same antibody or a different antibody to the detection antibody. Methods of labelling antibodies are known in the art. Suitable labels include fluorescent labels, or enzyme linked labels.

In one example, the method comprises contacting the biological sample with a labelled aptamer.

In some examples, if the biomarkers are polynucleotides, then the analysis method may comprise detecting and/or measuring a gene transcript corresponding to an individual biomarker. In one example, the transcript is detected using an oligonucleotide probe, in another, by high throughput RNA sequencing. Such methods will be familiar to those in the art.

In one example, the methods of the disclosure comprise performing a linear logistic regression analysis using the base-10 logarithms of the biomarker concentration. In one example, the methods of the disclosure comprise performing a linear logistic regression analysis using the base-2 logarithms of the biomarker concentration. In one example, the analysis is a Bayesian probability algorithm. Other analytic procedures may include logistic regression, adaptive index modelling, partial least squares discriminant analysis, feature vector (weighted and unweighted) and random forest. Re-sampling and cross validation may be used to control bias and provide confidence intervals of sensitivity.

The methods of the present disclosure can be used to identify subjects with APA. In some examples, a disease score is obtained for a subject which may be used to assess whether the subject requires further investigation. Examples of such investigations include colonoscopy, sigmoidoscopy, CT colonography or barium enema etc.

In some examples, the subject is identified as requiring administration of a therapeutic agent, such as after a confirmatory colonoscopy. Examples of such therapeutic agents include chemopreventatives or hormone regulating agents. In one example, the therapeutic agent is a combination of erlotinib (Tarceva) and sulindac (Aflodac).

In some examples, the subject is identified as requiring administration of chemotherapy and/or radiotherapy. In some examples, the methods of treatment described herein comprise administering a therapeutic agent (e.g., a chemotherapeutic/chemopreventative or hormone regulating agent) or radiotherapy, such as after a confirmatory colonoscopy.

In some examples, the subject is identified as requiring surgical resection. Accordingly, in some examples, the methods of the disclosure comprise a step of surgical resection.

In another example, the method further describes obtaining a biological sample from the subject. In a further example, the biological sample is a blood sample. In another example, the sample is a serum or plasma sample. In one example, the biological sample is a urine sample or any other bodily fluid in which the APA biomarkers can be detected and/or measured e.g. faeces.

In some examples the method further comprises obtaining a biological sample from a subject, more particularly an APA subject. Methods of obtaining a biological sample will be known to those skilled in the art. For example, for the extraction of a blood sample, it is preferred that a venous or arterial draw is performed.

In one example, the subject according to any aspect has no symptoms or family history of adenoma or polyps of the colon.

In some examples, the subject according to any aspect has previously received a FOBT. In certain examples, the subject according to any aspect has a positive diagnosis of adenoma, cancer or polyps based on FOBT. In another example, the subject according to any aspect has a negative diagnosis of adenoma, cancer or polyps based on FOBT.

In another example, the subject has a hereditary or other condition that increases their risk of developing polyps and/or adenomas. In one example, the subject has familial adenomatous polyposis (FAP).

In another example, the subject has previously been treated for polyps or adenomas.

In some examples, the methods described herein can be used for the diagnosis, prediction, prognosis and/or monitoring of polyp and/or adenoma formation in a subject.

In a fifth aspect, the disclosure provides a method of screening a subject to identify whether the subject requires further investigation by diagnostic colonoscopy or sigmoidoscopy, comprising:
(i) performing the method according to the fourth aspect of the disclosure; and
(ii) based on the disease score obtained, providing a recommendation for definitive diagnosis by colonoscopy or sigmoidoscopy.

In one example, the method detects biomarker combinations set forth herein. In another example, the method detects biomarker combinations set forth in any one of Tables 6 to 29.

Preferably the subject according to the methods described herein is human. In one example, the subject being screened has one or more risk factors for APA including, but not limited to being over 50 years of age, being overweight, having a family history of adenoma or colorectal adenocarcinoma, having ovarian or uterine cancer before the age of 50, having Crohn's disease or ulcerative colitis, having type 2 diabetes or having a hereditary disorder such as Lynch syndrome, Gardner's syndrome, familial adenomatous polyposis (FAP), MYH-associated polyposis (MAP) syndrome and serrated polyposis syndrome.

The subject according to any aspect described herein may or may not have colorectal cancer (CRC). In one example, the subject does not have CRC.

In some examples, the subject presents with one or more of the following: rectal bleeding, change in stool colour, change in bowel habits, abdominal pain and/or iron deficiency anaemia.

In a sixth aspect, the disclosure provides a composition comprising labelled antibodies that specifically bind to the biomarkers in a biomarker panel as described herein.

In one example, each compound individually binds to a biomarker. In one example, the compounds are antibodies.

In one example, the compounds bind to a biomarker combination set forth in any one of Tables 6 to 28. In one example, the compounds bind to a biomarker combination set forth in any one of Tables 13 to 28. In one example, the compounds bind to a biomarker combination set forth in any one of Tables 6 to 28 and 31 to 33.

The disclosure also provides a composition when used for identifying a subject at risk of APA, the composition comprising one or more (e.g. two or more, three or more, four or more, or five or more) labelled compounds that specifically binds to the biomarkers within a biomarker panel as described herein. In one example, the composition comprises five labelled compounds.

The disclosure also provides a composition for identifying a subject at risk of APA, the composition comprising one or more (e.g. two or more, three or more, four or more, or five or more) labelled compounds that specifically binds to the biomarkers within a biomarker panel as described herein. In one example, the composition comprises five labelled compounds.

In some examples, the methods identify a subject at risk of APA.

In a seventh aspect, the disclosure provides a kit for detecting APA in a subject comprising:
(i) one or more compounds that specifically bind to the biomarkers in a biomarker panel as described herein;
(ii) optionally one or more labelled probes that specifically bind to the biomarkers;
(iii) optionally a detection reagent for detecting binding of the one or more labelled probes and/or the one or more compounds to the biomarkers; and
(iv) optionally instructions for use.

In some examples, the kit further comprises a container for receiving a biological sample from the subject.

In one example, each of the one or more compounds individually binds to each of the biomarkers.

In one example, the one or more compounds are antibodies.

In some examples, the one or more compounds are labelled.

In alternative examples, the one or more compounds are not labelled.

In one example, the compounds bind to a biomarker combination set forth as described herein. In another example, the compounds bind to a biomarker combination set forth in any one of Tables 6 to 28. In one example, the compounds bind to a biomarker combination set forth in any one of Tables 6 to 28 and 31 to 33.

In particular examples, the compounds are coupled, bound, affixed or otherwise linked to a substrate.

In one example, the kit further comprises an ELISA plate to which the one or more compounds are coupled. In one particular example, the kit comprises an ELISA plate on which is immobilised capture antibodies corresponding to IGFBP2 and one or more, two or more, three or more, four or more, five or more, or six or more biomarkers selected from the group consisting of DKK-3, tumour M2PK, Mac2BP, TGFβ1, TIMP1, IL-8, IL-13 and EpCAM. In one example, the ELISA plate further comprises immobilised capture antibodies corresponding to BDNF.

In another example, the kit comprises a bead to which the one or more compounds are coupled. In one specific example, the kit further comprises a bead on which is immobilised capture antibodies corresponding to IGFBP2 and one or more, two or more, three or more, four or more, five or more, or six or more biomarkers selected from the group consisting of DKK-3, tumour M2PK, Mac2BP, TGFβ1, TIMP1, IL-8, IL-13 and EpCAM, or corresponding to a biomarker panel as described herein. In one example, the bead further comprises immobilised capture antibodies corresponding to BDNF.

In some examples, the kit comprises a membrane to which the one or more compounds are coupled. In one specific example, the kit further comprises a membrane on which is immobilised capture antibodies corresponding to IGFBP2 and one or more, two or more, three or more, four or more, five or more, or six or more biomarkers selected from the group consisting of DKK-3, tumour M2PK, Mac2BP, TGFβ1, TIMP1, IL-8, IL-13 and EpCAM, or corresponding to a biomarker panel as described herein. In one example, the membrane further comprises immobilised capture antibodies corresponding to BDNF.

In some examples, the kit also provides instructions for the analysis of the detected biomarkers by computer generated algorithms. In a further example, a clinical report is generated.

Suitably, the kit is for use in the method of any one of the first, second, third, fourth, fifth or seventh aspects.

In an eighth aspect, the disclosure provides a method of treating a subject, the method comprising:
(i) performing the method according to the fourth or fifth aspect to obtain a disease score for the subject's risk of APA;
(ii) administering to the subject one or more of colonoscopy with concomitant polypectomy or referral for surgical polyp removal.

In a ninth aspect, the disclosure provides a method for detecting the presence and/or level of protein biomarkers in a subject suspected of having APA or a patient having APA, the method comprising:
(a) providing a blood, plasma or serum sample obtained from the subject or the patient;
(b) contacting the sample with antibodies that specifically bind to IGFBP2 and one or more protein biomarkers in the sample, wherein the one or more protein biomarkers are selected from the group consisting of DKK-3, tumour M2PK, Mac2BP, TGFβ1, TIMP1, IL-8, IL-13 and EpCAM or wherein the protein biomarkers comprise a panel of biomarkers as described herein; and
(c) detecting antibody binding to the protein biomarkers, thereby detecting the presence and/or level of the biomarkers.

In one example, the one or more protein biomarkers comprise BDNF and IGFBP2.

In one example, the present method further includes the step of contacting the antibodies with secondary antibodies that are detectably labelled.

In one example, the detecting step (c) detects the protein biomarkers in the sample of the subject or the patient with a sensitivity of at least 30% and a specificity of at least 86%.

In a tenth aspect, there is provided a method for the detection of colorectal pre-cancerous adenomas (APA) in a subject, the method comprising:
determining a measurement for a panel of biomarkers in a biological sample obtained from the subject, the panel of biomarkers selected from any one of those in Tables 6 to 28 herein;
wherein the measurement comprises measuring a level of each of the biomarkers in the panel.

It will be understood that, in addition to age and/or gender, the present disclosure encompasses additional demographic or morphometric terms which are not specifically described herein. Examples of these other demographic or morphometric terms include, but are not limited to, smoking history, body mass index (BMI) and hip to waist ratio.

In examples of the first, second, third and further aspects described herein, the biomarker panel does not include TFF3 (Trefoil factor 3). In one example, the biomarker panel does not include Flt3L (Fms-related tyrosine kinase 3 ligand). In one example, the biomarker panel does not include TFF3 and FIt3L.

Suitably, the present method further comprises one or more features according to the methods of the above aspects.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

The term “and/or”, e.g., “X and/or Y” shall be understood to mean either “X and Y” or “X or Y” and shall be taken to provide explicit support for both meanings or for either meaning.

As used herein, the terms “a”, “an” and “the” include both singular and plural aspects, unless the context clearly indicates otherwise.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, 3rd edn, Cold Spring Harbour Laboratory Press (2001), R. Scopes, Protein Purification—Principals and Practice, 3rd edn, Springer (1994), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “APA” refers to “advanced pre-cancerous adenoma(s)”. This term includes “advanced colorectal adenomas” or “advanced adenoma”. As used herein, APA is also understood to include “adenomatous polyps” of the type that originate in the caecum, colon or rectum and which have the potential to develop into colorectal cancer. Broadly speaking, adenomatous polyps can range in diameter from less than 5 mm (diminutive) to over 30 mm (giant) and are typically <10 mm in diameter. They include polyps with low grade dysplasia (mildly abnormal) and those with high-grade dysplasia (abnormal in appearance) Adenomatous polyps falling within the APA description are typically benign and appear at the precancerous stage on the developmental pathway of colorectal cancer but have a higher risk of progressing to a cancerous/metastatic stage than other adenomatous polyps. APA may be defined or diagnosed by the protocol or features described in Imperiale et al. (N Engl J Med 2014; 370:1287-97), which is incorporated by reference herein. APA as used herein can be described as advanced precancerous lesions of the colorectal mucosa with high-grade dysplasia (any size) or with ≥20% villous histologic features (any size) or measuring ≥10 mm in the greatest dimension and including sessile serrated polyps measuring 10 mm or more in the greatest dimension. APA may also include circumstances where 3 or more adenomas ≥5 mm and <10 mm in their longest dimension are contemporaneously present at colonoscopy. A polyp is defined as an abnormal growth of tissue projecting from a mucous membrane sometimes attached to the surface by a narrow, elongated stalk. As used herein, the term APA includes sessile adenoma (having a flattened, broad based appearance by histology) sessile serrated adenoma and serrated adenoma (having a saw-tooth appearance by histology). The term APA is also understood to encompass tubular adenoma greater than 10 mm in its longest dimension, tubulovillus adenoma of any size where the villous content is >20% and villous adenoma of any size. Tubular adenomas are the most common of the adenomatous polyps and they are the least likely of colon polyps to develop into colon cancer. Tubulovillous adenoma is yet another type. Villous adenomas are a third type that is normally larger in size than the other two types of adenomas and they are associated with the highest morbidity and mortality rates of all polyps. For the avoidance of doubt, APA includes advanced adenomas.

An “APA subject” as used herein refers to a subject who is suspected of having advanced precancerous adenomas of the type described above, including a subject presenting with one or more risk factors that contribute to the formation of adenomas as described herein. An APA subject also includes a subject with a known hereditary disorder that increases the probability of colon polyp formation. The APA subject may or may not have CRC. In one example, the APA subject does not have CRC.

The term, “biomarker” as used herein, refers to any biological compound that can be measured as an indicator of the physiological status of a biological system. In some examples, the biomarker is a polynucleotide or nucleic acid. In some examples, the biomarker is a polypeptide or protein.

As used herein, the term “biological sample” refers to any material in which a biomarker as described herein can be detected. A biological sample can refer to a cell or population of cells or a quantity of tissue or fluid from a subject. Preferably, the sample is obtained from the subject so that the detection of biomarkers can be performed in vitro. Alternatively, biomarker detection may occur in vivo. The sample can be used as obtained directly from the source or following at least one step of (partial) purification. The sample can be prepared in any convenient medium which does not interfere with the methods described herein. Typically, the sample is in an aqueous solution, biological fluid, cells or tissue. Preferably, the sample is whole blood, plasma, lymph or serum. Methods of sample preparation can include filtration, distillation, separation, concentration, inactivation of interfering components and the addition of reagents.

The terms “polypeptide,” “peptide” and “protein” are used interchangeably herein to refer to a polymer of amino acid residues. A polypeptide is a single linear polymer chain of amino acids bonded together by peptide bonds between the carboxyl and amino groups of adjacent amino acid residues. Polypeptides can be modified, e.g., by the addition of carbohydrate, phosphorylation, etc. The term polypeptide includes an antibody.

The term “immunoassay” is an assay that uses an antibody to specifically bind an antigen (e.g., a marker). The immunoassay is characterized by the use of specific binding properties of a particular antibody to isolate, target, and/or quantify the antigen.

The term “antibody” refers to a polypeptide ligand substantially encoded by an immunoglobulin gene or immunoglobulin genes, or fragments thereof, which specifically binds and recognizes an epitope. Antibodies exist, e.g., as intact immunoglobulins or as a number of well-characterized fragments produced by digestion with various peptidases. This includes, e.g., Fab″ and F(ab)″2 fragments. As used herein, the term “antibody” also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, but does not include the heavy chain variable region.

A “control sample” as referred to herein refers to a non-diseased, healthy condition that is used as a relative marker in which to study fluctuations or compare the normal non-diseased healthy condition, or it can also be used to calibrate or normalise values. Accordingly, in some examples, the control sample can be obtained from a subject determined to have substantially no colorectal lesions, such as colorectal cancer and advanced adenoma (e.g., a subject with no pathological findings at colonoscopy). In an alternative example, control samples are those obtained from persons with colorectal lesions other than colorectal cancer and advanced adenoma. Such control samples may include samples from persons who have non-advanced adenoma(s) (e.g., one or two adenomas ≥5 mm and <10 mm with low or no dysplasia and villous histological features <25% and one or two adenomas <5 mm in diameter with no advanced histological features) and/or non-neoplastic lesions (e.g., no polyps/adenomas but with diverticula disease or haemorrhoids present). In this regard, the present method may be utilised to identify the APA subject from subjects having non-advanced adenomas and/or subjects with substantially no colorectal lesions (e.g., subjects having no colorectal cancer, advanced adenoma and non-advanced adenoma).

The term “case sample” as referred to herein generally refers to those samples obtained or derived from subjects with a colorectal cancer, or particularly subjects with an advanced colorectal neoplasia, such as colorectal cancer and advanced adenoma, or more particularly, subjects with an advanced adenoma. In particular examples, the colorectal cancer or adenoma can be confirmed by colonoscopy. The case sample may or may not have colorectal cancer (CRC). In one example, the case sample does not have CRC.

The term “measurement” as used herein refers to assessing the presence, absence, quantity or amount of a given substance within a sample, including the derivation of qualitative or quantitative concentration levels of such substances. The term “measuring” means methods which include detecting the presence or absence of biomarker(s) in the sample, quantifying the amount of biomarker(s) in the sample, and/or qualifying the type of biomarker. Measuring can be accomplished by methods known in the art and those further described herein, including but not limited to mass spectrometry approaches and immunoassay approaches (e.g. ELISA, surface plasmon resonance) or any suitable methods can be used to detect and measure one or more of the biomarkers described herein.

The term “detect” refers to identifying the presence, absence or amount of the object (e.g. biomarker) to be detected. Non-limiting examples include, but are not limited to, detection of a DNA molecules, proteins, peptides, protein complexes, or RNA molecules. Detection of a biomarker can be accomplished by methods known in the art and those further described herein, including but not limited to mass spectrometry approaches and immunoassay approaches (e.g. ELISA).

The term “diagnosis” means identifying the presence or nature of a pathological condition, or a subtype of a pathologic condition i.e. presence or risk of colon polyps. Diagnostic methods differ in their sensitivity and specificity. Diagnostic methods may not provide a definitive diagnosis of a condition; however, it suffices if the method provides a positive indication that aids in diagnosis or provides an indication that a subject is at an increased risk of advanced adenoma of the colon.

The term “expression” as used herein refers, in one context to the presence of a biomarker protein on the cell surface that can be detected by a compound (e.g. antibody). In some examples, detecting the expression of a biomarker protein in a biological sample includes determining the concentration of that protein in the biological sample.

The term “subject,” refers to a vertebrate, preferably a mammal, more preferably a human. Mammals include, but are not limited to, murines, simians, farm animals, sport animals, and pets. Specific mammals include rats, mice, cats, dogs, monkeys, and humans. Non-human mammals include all mammals other than humans.

A “healthy subject” as described herein is taken to mean an individual who is known not to have APA or colon polyps as determined by colonoscopy for example. Healthy subjects also include subjects that may present with a low number of colonic polys (e.g. less than 3) that are small and not histologically threatening or in need of clinical intervention.

The term “prognosis” is used herein to refer to the prediction of the likelihood of disease, disease stage or disease progression, including recurrence and/or therapeutic response.

The term “prediction” is used herein to refer to the likelihood that a patient will have a particular clinical outcome, whether positive or negative. The predictive methods of the disclosure can be used clinically to make treatment decisions by choosing the most appropriate treatment modalities for any particular subject.

The term “report” refers to a printed or electronic result provided from the methods of the present disclosure to the physician. The report can indicate the likelihood of the presence of, nature of, or risk for the pathological condition. The report can also indicate what treatment is most appropriate e.g. no action, surgery, further tests, or administering therapeutic agents.

The term “mass spectrometry” as used herein refers to a gas phase ion spectrometer that measures a parameter that can be translated into mass-to charge (m/z) ratios of gas phase ions. Mass spectrometers generally include an ion source and a mass analyser. Examples of mass spectrometers are time-of-flight, magnetic sector, quadrupole filter, ion trap, ion cyclotron resonance, electrostatic sector analyser and hybrids of these. Mass spectrometry refers to the use of a mass spectrometer to detect gas phase ions. The use of mass spectrometry for biomarker detection is known in the art and is described, for example in C A Crutchfield et al., (2016) Clin Proteomics13:1).

The term “treating” as used herein may include administering a therapeutically effective amount of a compound sufficient to reduce the size of, or eliminate an adenoma or polyp as described herein. The term also includes polypectomy, being the surgical removal of a polyp.

### DETAILED DESCRIPTION

### General Overview

The present disclosure provides methods and compositions for the analysis of a biological sample from a subject using an assay coupled with an algorithm executable by a computer for determining a biomarker which is indicative of the likelihood or presence of advanced precancerous adenomas (APA). Generally, the methods use polynucleotides or polypeptides present in the biological sample of the subject to identify biomarkers or a biomarker profile of early-stage precancerous polyps or adenomas and thus identify subjects who may require further screening such as colonoscopy or sigmoidoscopy. In one example, the methods use polypeptides present in the biological sample of the subject.

The present disclosure also provides a commercial diagnostic kit that in general will include compositions used for the detection of biomarkers (e.g. protein biomarkers) provided herein. The kit may also include a report that indicates the risk of APA in a subject.

### Biomarkers and APA

The present disclosure provides biomarker panels for the detection of advanced pre-cancerous colorectal neoplasia, more particularly advanced pre-cancerous adenomas (APA) in a subject. In one example, the subject is a subject at risk of APA. In another example, the subject is one with no risk of APA.

A colon polyp is benign clump of cells that forms on the lining of the large intestine or colon. Almost all polyps are initially non-malignant. However, over time some can turn into cancerous lesions. The cause of most colon polyps is not known, but they are common in adults. For some people found to have colorectal polyps at colonoscopy, surveillance colonoscopy is recommended at three yearly intervals. This includes those presenting with a large adenoma (more than 1 cm in diameter), adenomas with high-grade dysplasia or villous change or multiple (3 or more) adenomas.

Currently, the most effective methods used for screening for polyps are highly invasive and expensive. Thus, despite the benefit of colonoscopy screening in the prevention and reduction of colon cancer, many of the people for whom the procedure is recommended decline to undertake it, primarily due to concerns about cost, discomfort, and adverse events. Less invasive screening tests such as the fecal occult blood test (FOBT), only detect advanced adenomas with very low sensitivity.

A simple blood test which helps classify the likelihood that a patient has a higher risk for the presence of APA may help physicians to guide patients' attitudes and actions regarding reluctance to undergo colonoscopy. Increased colonoscopy screening compliance would result in early detection and removal of pre-cancerous adenoma leading to a reduction in colon cancer cases and cancer-related morbidity and mortality.

The present disclosure provides for a biomarker blood test, which is less invasive than a colonoscopy, that will determine an individual's protein expression profile. In some examples of the disclosure, a report is generated based on the predicted likelihood an individual's polyp status and/or risk of developing colon polyps based on that profile. Thus, the present disclosure provides methods, compositions and kits, compositions that provide information for an individual's colon polyp status and/or risk of developing APA. The present disclosure utilises a panel of biomarkers measured in a biological sample obtained from a subject. In some examples, the biomarker is a protein biomarker. In other examples, the biomarker is a nucleic acid biomarker.

The biomarkers found to be useful in identifying subjects having APA or at greater risk of developing APA were IGFBP2, DKK-3, tumour M2PK, Mac2BP, TGFβ1, TIMP1, IL-8, IL-13 and EpCAM. Reference to the protein sequences for these biomarkers is found in Table 2. The biomarkers may optionally include BDNF. In one example, the biomarkers found to be useful in identifying subjects having APA or at greater risk of developing APA were BDNF, IGFBP2, DKK-3, tumour M2PK, Mac2BP, TGFβ1, TIMP1, IL-8, IL-13 and EpCAM. Reference to the protein sequences for BDNF is found in Table 2. Without wishing to be bound by theory, the inventors have found that the inclusion of BDNF as an additional biomarker may increase the sensitivity of detection. For example, when BDNF is combined with IGFB2, TIMP1, DKK3 and M2PK in a biomarker panel, the sensitivity of detection is comparable to, or greater than, that achieved with the fecal occult blood test (FOBT). Reference to any of these biomarkers includes reference to all polypeptide and polynucleotide variants such as isoforms and transcript variants as would be known by the person skilled in the art. As would be understood by the person skilled in the art, the biomarkers may also undergo processing (for example, to remove a signal sequence from a pro-form or other post-translational modification) to form a mature or processed polypeptide. In one example, the biomarker is the mature or processed polypeptide.

In some examples, the biomarker panel may include IGFB2 and 1, 2, 3, 4, 5, 6 or more biomarkers selected from the group consisting of DKK-3, tumour M2PK, Mac2BP, TGFβ1, TIMP1, IL-8, IL-13 and EpCAM, and optionally BDNF. In some examples, the biomarkers comprise IGFBP2 and BDNF. In some examples, the biomarker panel may include IGFB2, BDNF and 3 biomarkers selected from the group consisting of DKK-3, tumour M2PK, Mac2BP, TGFβ1, TIMP1, IL-8, IL-13 and EpCAM.

It will be understood that, in some examples, one or more demographic or morphometric terms may also be factored into the analysis, for example, using a logistic regression or other machine learning-derived algorithms. Demographic or morphometric terms, include but are not limited to, age, gender, smoking history, body mass index (BMI) and hip to waist ratio.

In some examples, the methods of the disclosure also contemplate the inclusion of the subject's gender as a biomarker in a biomarker panel described herein. In some examples, the methods of the disclosure also contemplate the inclusion of the subject's age as a biomarker in a biomarker panel described herein. In some examples, the methods of the disclosure also contemplate the inclusion of the subject's BMI as a biomarker in a biomarker panel described herein.

### Sample Preparation and Processing

Before analysing the biological sample, it may be desirable to perform one or more sample preparation operations upon the sample. Generally, these sample preparation operations may include such manipulations as extraction and isolation of intracellular material from a cell or tissue such as, the extraction of nucleic acids, protein, or other macromolecules from the samples.

Sample preparation which can be used with the methods of disclosure include but are not limited to, centrifugation, affinity chromatography, magnetic separation, fractionation, precipitation, and combinations thereof.

Sample preparation can further include dilution by an appropriate solvent and amount to ensure the appropriate range of concentration levels is detected by a given assay.

Accessing the nucleic acids and macromolecules from the intracellular space of the sample may generally be performed by either physical, chemical methods, or a combination of both. In some applications of the methods, following the isolation of the crude extract, it will often be desirable to separate the nucleic acids, proteins, cell membrane particles, and the like. In some examples of the methods it will be desirable to keep the nucleic acids with its proteins, and cell membrane particles.

In some examples of the methods provided herein, nucleic acids and proteins can be extracted from a biological sample prior to analysis using methods of the disclosure. Extraction can be by means including, but not limited to, the use of detergent lysates, sonication, or vortexing with glass beads.

In some examples, molecules can be isolated using any technique suitable in the art including, but not limited to, techniques using gradient centrifugation (e.g., cesium chloride gradients, sucrose gradients, glucose gradients, etc.), differential centrifugation protocols, boiling, purification kits, and the use of liquid extraction with agent extraction methods such as methods using Trizol or DNAzol.

Samples may be prepared according to standard biological sample preparation depending on the desired detection method. For example for mass spectrometry detection, biological samples obtained from a patient may be centrifuged, filtered, processed by immunoaffinity column, separated into fractions, partially digested, and combinations thereof. Various fractions may be resuspended in appropriate carrier media such as buffer or other types of loading solution for detection and analysis, including LCMS loading buffer.

### Biomarker Measurement

Measurement of a biomarker panel relates to a quantitative measurement of a plurality of biomarkers. The present disclosure provides for methods for detecting biomarkers in biological samples. Biomarkers can include but are not limited to proteins, DNA molecules, and RNA molecules. More specifically the present disclosure is based on the discovery of protein biomarkers that are differentially expressed in subjects that have an adenoma or an increased risk of advanced adenoma and thus an increased risk of progression to colorectal cancer. Therefore the detection of one or more of these differentially expressed biomarkers in a biological sample provides useful information whether or not a subject is at risk and what type of nature or state of the condition. Any suitable method known to the skilled person can be used to detect one or more of the biomarker described herein.

Useful analyte capture agents that can be used with the present disclosure include but are not limited to antibodies, such as crude serum containing antibodies, purified antibodies, monoclonal antibodies, polyclonal antibodies, synthetic antibodies, antibody fragments (for example, Fab fragments); antibody interacting agents, such as protein A, carbohydrate binding proteins, and other interactants; protein interactants (for example avidin and its derivatives); peptides; and small chemical entities, such as enzyme substrates, cofactors, metal ions/chelates, and haptens. Antibodies may be modified or chemically treated to optimize binding to targets or solid surfaces (e.g. biochips and columns).

In one particular example of the disclosure, the biomarker can be detected in a biological sample using an immunoassay. Immunoassays are assay that use an antibody that specifically bind to or recognizes an antigen (e.g. site on a protein or peptide, biomarker target). The method includes the steps of contacting the biological sample with the antibody and allowing the antibody to form a complex with the antigen in the sample, washing the sample and detecting the antibody-antigen complex with a detection reagent. In one example, antibodies that recognize the biomarkers may be commercially available. In another examples, an antibody that recognizes the biomarkers may be generated by known methods of antibody production.

Alternatively, the marker in the sample can be detected using an indirect assay, wherein, for example, a second, labelled antibody is used to detect bound marker-specific antibody. Exemplary detectable labels include magnetic beads (e.g., DYNABEADS™), fluorescent dyes, radiolabels, enzymes (e.g., horse radish peroxide, alkaline phosphatase and others commonly used), and colorimetric labels such as colloidal gold or coloured glass or plastic beads. The marker in the sample can be detected using and/or in a competition or inhibition assay wherein, for example, a monoclonal antibody which binds to a distinct epitope of the marker is incubated simultaneously with the mixture.

The conditions to detect an antigen using an immunoassay will be dependent on the particular antibody used. Also, the incubation time will depend upon the assay format, marker, volume of solution, concentrations and the like. In general, the immunoassays will be carried out at room temperature, although they can be conducted over a range of temperatures, such as 10 degrees to 40 degrees Celsius depending on the antibody used.

There are various types of immunoassay known in the art that, as a starting basis, can be used to tailor the assay for the detection of the biomarkers of the present disclosure. Useful assays can include, for example, an enzyme immune assay (EIA) such as enzyme-linked immunosorbent assay (ELISA), including the sandwich ELISA. There are many variants of these approaches, but all are based on a similar idea. For example, if an antigen can be bound to a solid support or surface, it can be detected by reacting it with a specific antibody and the antibody can be quantitated by reacting it with either a secondary antibody or by incorporating a label directly into the primary antibody. Alternatively, an antibody can be bound to a solid surface and the antigen added. A second antibody that recognizes a distinct epitope on the antigen can then be added and detected. This is frequently called a ‘sandwich assay’ and can frequently be used to avoid problems of high background or non-specific reactions. These types of assays are sensitive and reproducible enough to measure low concentrations of antigens in a biological sample.

Immunoassays can be used to determine presence or absence of a marker in a sample as well as the quantity of a marker in a sample. Methods for measuring the amount of, or presence of, antibody-marker complex include but are not limited to, fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). In general these regents are used with optical detection methods, such as various forms of microscopy, imaging methods and non-imaging methods. Electrochemical methods include voltammetry, amperometry and elecrochemiluminescence methods. Radio frequency methods include multipolar resonance spectroscopy. PCR methods include Proximity Extension Assay (PEA).

In one example, the disclosure can use antibodies for the detection of the biomarkers. Antibodies can be made that specifically bind to the biomarkers of the present assay can be prepared using standard methods known in the art. For example polyclonal antibodies can be produced by injecting an antigen into a mammal, such as a mouse, rat, rabbit, goat, sheep, or horse for large quantities of antibody. Blood isolated from these animals contains polyclonal antibodies—multiple antibodies that bind to the same antigen. Alternatively polyclonal antibodies can be produced by injecting the antigen into chickens for generation of polyclonal antibodies in egg yolk. In addition, antibodies can be made that specifically recognize modified forms for the biomarkers such as a phosphorylated form of the biomarker, that is to say, they will recognize a tyrosine or a serine after phosphorylation, but not in the absence of phosphate. In this way antibodies can be used to determine the phosphorylation state of a particular biomarker.

Antibodies can be obtained commercially or produced using well-established methods. To obtain antibody that is specific for a single epitope of an antigen, antibody-secreting lymphocytes are isolated from the animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies.

Polyclonal and monoclonal antibodies can be purified in several ways. For example, one can isolate an antibody using antigen-affinity chromatography which the antigen is coupled to bacterial proteins such as Protein A, Protein G, Protein L or the recombinant fusion protein, Protein A/G followed by detection via UV light at 280 nm absorbance of the eluate fractions to determine which fractions contain the antibody. Protein A/G binds to all subclasses of human IgG, making it useful for purifying polyclonal or monoclonal IgG antibodies whose subclasses have not been determined. In addition, it binds to IgA, IgE, IgM and (to a lesser extent) IgD. Protein A/G also binds to all subclasses of mouse IgG but does not bind mouse IgA, IgM or serum albumin. This feature, allows Protein A/G to be used for purification and detection of mouse monoclonal IgG antibodies, without interference from IgA, IgM and serum albumin.

Antibodies can be derived from different classes or isotypes of molecules such as, for example, IgA, IgD, IgE, IgM and IgG. IgA are designed for secretion into bodily fluids while others, like the IgM are designed to be expressed on the cell surface. The antibody that is most useful in biological studies is the IgG class, a protein molecule that is made and secreted and can recognize specific antigens. The IgG is composed of two subunits including two “heavy” chains and two “light” chains. These are assembled in a symmetrical structure and each IgG has two identical antigen recognition domains. The antigen recognition domain is a combination of amino acids from both the heavy and light chains. The molecule is roughly shaped like a “Y” and the arms/tips of the molecule comprise the antigen-recognizing regions or Fab (fragment, antigen binding) region, while the stem of Fc (Fragment, crystallizable) region is not involved in recognition and is fairly constant. The constant region is identical in all antibodies of the same isotype, but differs in antibodies of different isotypes.

It is also possible to use an antibody to detect a protein after fractionation by western blotting. In one example, the disclosure can use western blotting for the detection of the biomarkers. Western blot (protein immunoblot) is an analytical technique used to detect specific proteins in the given sample or protein extract from a sample. It uses gel electrophoresis, SDS-PAGE to separate either native proteins by their 3-dimensional structure or it can be run under denaturing conditions to separate proteins by their length. After separation by gel electrophoresis, the proteins are then transferred to a membrane (typically nitrocellulose or PVDF). The proteins transferred from the SDS-PAGE to a membrane can then be incubated with particular antibodies under gentle agitation, rinsed to remove non-specific binding and the protein-antibody complex bound to the blot can be detected using either a one-step or two step detection methods. The one step method includes a probe antibody which both recognizes the protein of interest and contains a detectable label, probes which are often available for known protein tags. The two-step detection method involves a secondary antibody that has a reporter enzyme or reporter bound to it. With appropriate reference controls, this approach can be used to measure the abundance of a protein.

In one example, the method of the disclosure can use flow cytometry. Flow cytometry is a laser based, biophysical technology that can be used for biomarker detection, quantification (cell counting) and cell isolation. This technology is routinely used in the diagnosis of health disorders, especially blood cancers. In general, flow cytometry works by suspending single cells in a stream of fluid, a beam of light (usually laser light) of a single wavelength is directed onto the stream of liquid, and the light scattering caused by the passing cell is detected by an electronic detection apparatus. Fluorescence-activated cell sorting (FACS) is a specialized type of flow cytometry that often uses the aid of florescent-labelled antibodies to detect antigens on cell of interest. This additional feature of antibody labelling use in FACS provides for simultaneous multiparametric analysis and quantification based upon the specific light scattering and fluorescent characteristics of each florescent-labelled cell and it provides physical separation of the population of cells of interest just as efficiently as traditional flow cytometry does.

In another example, the flow cytometry is combined with bead systems, wherein the target antigen is attached to a bead. Such systems are known to persons skilled in the art.

A wide range of fluorophores can be used as labels in flow cytometry. Fluorophores are typically attached to an antibody that recognizes a target feature on or in the cell. Examples of suitable fluorescent labels include, but are not limited to: fluorescein (FITC), 5,6-carboxymethyl fluorescein, Texas red, nitrobenz-2-oxa-1,3-diazol-4-yl (NBD), and the cyanine dyes Cy3, Cy3.5, Cy5, Cy5.5 and Cy7. Other Fluorescent labels such as Alexa Fluor® dyes, DNA content dye such as DAPI and Hoechst dyes are well known in the art and all can be easily obtained from a variety of commercial sources. Each fluorophore has a characteristic peak excitation and emission wavelength, and the emission spectra often overlap. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus choosing ones that do not have a lot of spectra overlap allows their simultaneous detection. The fluorescent labels can be obtained from a variety of commercial sources. The maximum number of distinguishable fluorescent labels is thought to be around approximately 17 or 18 different fluorescent labels. This level of complex read-out necessitates laborious optimization to limit artefacts, as well as complex deconvolution algorithms to separate overlapping spectra. Quantum dots are sometimes used in place of traditional fluorophores because of their narrower emission peaks. Other methods that can be used for detecting include isotope labelled antibodies, such as lanthanide isotopes. However this technology ultimately destroys the cells, precluding their recovery for further analysis.

In one example, the method of the disclosure can use immunohistochemistry for detecting the expression levels of the biomarkers of the present disclosure. Thus, antibodies specific for each marker are used to detect expression of the claimed biomarkers in a biological sample. The antibodies can be detected by direct labelling of the antibodies themselves, for example, with radioactive labels, fluorescent labels, hapten labels such as, biotin, or an enzyme such as horse radish peroxidase or alkaline phosphatase. Alternatively, unlabelled primary antibody is used in conjunction with a labelled secondary antibody, comprising antisera, polyclonal antisera or a monoclonal antibody specific for the primary antibody. Immunohistochemistry protocols are well known in the art and protocols and antibodies are commercially available. Alternatively, one could make an antibody to the biomarkers or modified versions of the biomarker or binding partners as disclosed herein that would be useful for determining the expression levels of in a biological sample.

In an example, the binding agents or compounds for detecting the biomarkers described herein are coupled, bound, affixed or otherwise linked to a substrate. To this end, the first and/or second binding agents disclosed herein can be coupled, bound, affixed or otherwise linked to a substrate that may be a bead, a matrix, a cross-linked polymer, a gel, a particle, a surface, a plate, a membrane, a well or other solid or semi-solid substrate. In a particular example, the substrate comprises one or more of a sensor chip surface (e.g., for BIACore or surface plasmon resonance), a biochip, an ELISA plate, a sepharose, an agarose, Protein A, Protein G, a magnetic bead or a paramagnetic particle, a nitrocellulose membrane, a PVDF membrane, or other substrate known to those skilled in the art. It is envisaged that the binding agents described herein can be formulated as discrete agents, such as in separate channels, chambers, wells or the like of a substrate.

In one example, the method of the disclosure can use a biochip. Biochips can be used to screen a large number of macromolecules. In this technology macromolecules are attached to the surface of the biochip in an ordered array format. The grid pattern of the test regions allowed analysed by imaging software to rapidly and simultaneously quantify the individual analytes at their predetermined locations (addresses). The CCD camera is a sensitive and high-resolution sensor able to accurately detect and quantify very low levels of light on the chip.

Biochips can be designed with immobilized nucleic acid molecules, full-length proteins, antibodies, affibodies (small molecules engineered to mimic monoclonal antibodies), aptamers (nucleic acid-based ligands) or chemical compounds. A chip could be designed to detect multiple macromolecule types on one chip. For example, a chip could be designed to detect nucleic acid molecules, proteins and metabolites on one chip. The biochip is used and designed to simultaneously analyze a panel biomarker in a single sample, producing a subjects profile for these biomarkers. The use of the biochip allows for the multiple analyses to be performed reducing the overall processing time and the amount of sample required.

Protein microarrays are a particular type of biochip which can be used with the present disclosure. The chip consists of a support surface such as a glass slide, nitrocellulose membrane, bead, or microtitre plate, to which an array of capture proteins are bound in an arrayed format onto a solid surface. Protein array detection methods must give a high signal and a low background. Detection probe molecules, typically labelled with a fluorescent dye, are added to the array. Any reaction between the probe and the immobilized protein emits a fluorescent signal that is read by a laser scanner. Such protein microarrays are rapid, can be automated, and offer high sensitivity of protein biomarker read-outs for diagnostic tests. However, it would be immediately appreciated to those skilled in the art that there is a variety of detection methods that can be used with this technology.

There are at least three types of protein microarrays that are currently used to study the biochemical activities of proteins. For example there are analytical microarrays (also known as capture arrays), Functional protein microarrays (also known as target protein arrays) and Reverse phase protein microarrays (RPA).

The present disclosure provides for the detection of the biomarkers using an analytical protein microarray, such as Luminex xMAP Technology. Analytical protein microarrays are constructed using a library of antibodies, aptamers or affibodies. The array is probed with a complex protein solution such as a blood, serum or a cell lysate that function by capturing protein molecules they specifically bind to. Analysis of the resulting binding reactions using various detection systems can provide information about expression levels of particular proteins in the sample as well as measurements of binding affinities and specificities. This type of protein microarray is especially useful in comparing protein expression in different samples.

In one example, the method of the disclosure can use functional protein microarrays. These are constructed by immobilising large numbers of purified full-length functional proteins or protein domains and are used to identify protein-protein, protein-DNA, protein-RNA, protein-phospholipid, and protein-small molecule interactions, to assay enzymatic activity and to detect antibodies and demonstrate their specificity. These protein microarray biochips can be used to study the biochemical activities of the entire proteome in a sample.

In one example, the method of the disclosure can use reverse phase protein microarrays (RPA). Reverse phase protein microarrays are constructed from tissue and cell lysates that are arrayed onto the microarray and probed with antibodies against the target protein of interest. These antibodies are typically detected with chemiluminescent, fluorescent or colorimetric assays. In addition to the protein in the lysate, reference control peptides are printed on the slides to allow for protein quantification. RPAs allow for the determination of the presence of altered proteins or other agents that may be the result of disease and present in a diseased cell.

In some examples detection of biomarkers utilises the ARCHITECT system (Abbott).

The present disclosure provides for the detection of the biomarkers using mass spectroscopy (alternatively referred to as mass spectrometry). Mass spectrometry (MS) is an analytical technique that measures the mass-to-charge ratio of charged particles. It is primarily used for determining the elemental composition of a sample or molecules, and for elucidating the chemical structures of molecules, such as peptides and other chemical compounds. MS works by ionizing chemical compounds to generate charged molecules or molecule fragments and measuring their mass-to-charge ratios. MS instruments typically consist of three modules (1) an ion source, which can convert gas phase sample molecules into ions (or, in the case of electrospray ionization, move ions that exist in solution into the gas phase) (2) a mass analyser, which sorts the ions by their masses by applying electromagnetic fields and (3) detector, which measures the value of an indicator quantity and thus provides data for calculating the abundances of each ion present.

Suitable mass spectrometry methods to be used with the present disclosure include but are not limited to, one or more of electrospray ionization mass spectrometry (ESI-MS), ESI-MS/MS, ESI-MS/(MS) n, matrix-assisted laser desorption ionization time-of-flight mass spectrometry (MALDI-TOF-MS), surface-enhanced laser desorption/ionization time-of-flight mass spectrometry (SELDI-TOF-MS), tandem liquid chromatography-mass spectrometry (LC-MS/MS) mass spectrometry, desorption/ionization on silicon (DIOS), secondary ion mass spectrometry (SIMS), quadrupole time-of-flight (Q-TOF), atmospheric pressure chemical ionization mass spectrometry (APCI-MS), APCI-MS/MS, APCI-(MS), atmospheric pressure photoionization mass spectrometry (APPI-MS), APPI-MS/MS, and APPI-(MS) n, quadrupole mass spectrometry, Fourier transform mass spectrometry (FTMS), and ion trap mass spectrometry, where n is an integer greater than zero.

To gain insight into the underlying proteomics of a sample, LC-MS is commonly used to resolve the components of a complex mixture. The LC-MS method generally involves protease digestion and denaturation (usually involving a protease, such as trypsin and a denaturant, such as urea, to denature tertiary structure and iodoacetamide to cap cysteine residues) followed by LC-MS with peptide mass fingerprinting or LC-MS/MS (tandem MS) to derive sequence of individual peptides. LC-MS/MS is most commonly used for proteomic analysis of complex samples where peptide masses may overlap even with a high-resolution mass spectrometer. Samples of complex biological fluids like human serum may be first separated on an SDS-PAGE gel or HPLC-SCX and then run in LC-MS/MS allowing for the identification of over 1000 proteins.

While multiple mass spectrometric approaches can be used with the methods of the disclosure as provided herein, in some applications it may be desired to quantify proteins in biological samples from a selected subset of proteins of interest. One such MS technique that can be used with the present disclosure is Multiple Reaction Monitoring Mass Spectrometry (MRM-MS), or alternatively referred to as Selected Reaction Monitoring Mass Spectrometry (SRM-MS).

The MRM-MS technique uses a triple quadrupole (QQQ) mass spectrometer to select a positively charged ion from the peptide of interest, fragment the positively charged ion and then measure the abundance of a selected positively charged fragment ion. This measurement is commonly referred to as a transition.

In some applications the MRM-MS is coupled with High-Pressure Liquid Chromatography (HPLC) and more recently Ultra High-Pressure Liquid Chromatography (UHPLC). In other applications MRM-MS is coupled with UHPLC with a QQQ mass spectrometer to make the desired LC-MS transition measurements for all of the peptides and proteins of interest.

In some applications the utilization of a quadrupole time-of-flight (qTOF) mass spectrometer, time-of-flight (TOF-TOF) mass spectrometer, Orbitrap mass spectrometer, quadrupole Orbitrap mass spectrometer or any Quadrupolar lon Trap mass spectrometer can be used to select for a positively charged ion from one or more proteins of interest. The fragmented, positively charged ions can then be measured to determine the abundance of a positively charged ion for the quantitation of the peptide or protein of interest.

In some applications the utilization of a time-of-flight (TOF), quadrupole time-of-flight (qTOF) mass spectrometer, time-of-flight (TOF-TOF) mass spectrometer, Orbitrap mass spectrometer or quadrupole Orbitrap mass spectrometer can be used to measure the mass and abundance of a positively charged peptide ion from the protein of interest without fragmentation for quantitation. In this application, the accuracy of the analyte mass measurement can be used as selection criteria of the assay. An isotopically labelled internal standard of a known composition and concentration can be used as part of the mass spectrometric quantitation methodology.

In some applications, time-of-flight (TOF), quadrupole time-of-flight (qTOF) mass spectrometer, time-of-flight (TOF-TOF) mass spectrometer, Orbitrap mass spectrometer or quadrupole Orbitrap mass spectrometer can be used to measure the mass and abundance of a protein of interest for quantitation. In this application, the accuracy of the analyte mass measurement can be used as selection criteria of the assay. Optionally this application can use proteolytic digestion of the protein prior to analysis by mass spectrometry. An isotopically labelled internal standard of a known composition and concentration can be used as part of the mass spectrometric quantitation methodology.

In some applications, various ionization techniques can be coupled to the mass spectrometers provided herein to generate the desired information. Non-limiting exemplary ionization techniques that can be used with the present disclosure include but are not limited to Matrix Assisted Laser Desorption Ionization (MALDI), Desorption Electrospray Ionization (DESI), Direct Assisted Real Time (DART), Surface Assisted Laser Desorption Ionization (SALDI), or Electrospray Ionization (ESI).

In some applications, HPLC and UHPLC can be coupled to a mass spectrometer a number of other protein separation techniques can be performed prior to mass spectrometric analysis. Some exemplary separation techniques which can be used for separation of the desired analyte (e.g., peptide or protein) from the matrix background include but are not limited to Reverse Phase Liquid Chromatography (RP-LC) of proteins or peptides, offline Liquid Chromatography (LC) prior to MALDI, 1 dimensional gel separation, 2-dimensional gel separation, Strong Cation Exchange (SCX) chromatography, Strong Anion Exchange (SAX) chromatography, Weak Cation Exchange (WCX), and Weak Anion Exchange (WAX). One or more of the above techniques can be used prior to mass spectrometric analysis.

In one example of the disclosure the biomarker can be detected in a biological sample using a microarray. Differential gene expression can also be identified, or confirmed using the microarray technique. Thus, the expression profile biomarkers can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. The source of mRNA typically is total RNA isolated from a biological sample, and corresponding normal tissues or cell lines may be used to determine differential expression.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labelled cDNA probes may be generated through incorporation of fluorescent nucleotides by reverse transcription of RNA extracted from tissues of interest. Labelled cDNA probes applied to the chip hybridize with specificity to each spot of DNA on the array. After stringent washing to remove non-specifically bound probes, the microarray chip is scanned by a device such as, confocal laser microscopy or by another detection method, such as a CCD camera. Quantitation of hybridization of each arrayed element allows for assessment of corresponding mRNA abundance. With dual colour fluorescence, separately labelled cDNA probes generated from two sources of RNA are hybridized pair-wise to the array. The relative abundance of the transcripts from the two sources corresponding to each specified gene is thus determined simultaneously. Microarray analysis can be performed by commercially available equipment, following manufacturer's protocols.

In one example of the disclosure, the biomarker can be detected in a biological sample using qRT-PCR, which can be used to compare mRNA levels in different sample populations, in normal and tumor tissues, with or without drug treatment, to characterize patterns of gene expression, to discriminate between closely related mRNAs, and to analyse RNA structure. The first step in gene expression profiling by RT-PCR is extracting RNA from a biological sample followed by the reverse transcription of the RNA template into cDNA and amplification by a PCR reaction. The reverse transcription reaction step is generally primed using specific primers, random hexamers, or oligo-dT primers, depending on the goal of expression profiling. The two commonly used reverse transcriptases are avilo myeloblastosis virus reverse transcriptase (AMV-RT) and Moloney murine leukemia virus reverse transcriptase (MLV-RT).

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan™ PCR typically utilizes the 5′-nuclease activity of Taq or Tth polymerase to hydrolyse a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labelled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700 Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system includes software for running the instrument and for analysing the data. 5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and Beta-Actin.

A more recent variation of the RT-PCR technique is the real time quantitative PCR, which measures PCR product accumulation through a dual-labelled fluorigenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996). Other quantitative methods include digital droplet PCR.

In a further example of the disclosure, the biomarker can be detected in a biological sample using RNA-Seq, to compare levels of RNAs in different sample populations, in normal and tumor/APA tissues, with or without drug treatment. RNA-Seq also facilitates identification of alternative gene spliced transcripts, including the definition of intron/exon boundaries and maintenance of or alterations to 5′ and 3′ ends of transcripts, post-transcriptional modifications to the bases, gene fusions and mutations/single nucleotide polymorphisms in mRNAs. It can also be used to identify variations in different populations of RNA including total RNA, small RNA, such as miRNA or tRNAs, and for ribosomal RNA profiling. RNA-Seq can be applied to RNA extracted from fresh or frozen tissue samples, e.g. tumour and adjacent normal tissue, single cells and in situ sequencing of fixed tissues as well as cells, micro-vesicles/exosomes and cell-free RNA present in blood and other bodily fluids including urine, faeces, cerebrospinal fluid and interstitial fluids.

In its most common format, RNA-Seq involves the isolation if RNA from the tissues or cell(s) or biological fluids of interest, removal of contaminating DNA using DNase, quality assessment of the RNA by gel or capillary electrophoresis and optionally, sub-selection of the RNA to be analysed. Sub-selection may include oligo dT selection to produce populations of RNAs enriched for mRNAs (the poly A containing fraction that binds to the oligo dT-containing immobilised phase (typically magnetic beads)) or enriched for ribosomal and other non-poly adenylated RNA species, enrichment for specific sequences through hybridisation selection or enrichment for small RNA targets, such as micro RNAs by size selection procedures. These latter can include sucrose/glycerol gradient centrifugation, passage through size-exclusion gels, selection via magnetic beads or using commercial kits. RNAs are converted to cDNA using reverse transcriptase as previously described. Fragmentation and size selection, performed to allow the preparation of sequences of a length suitable for the sequencing machine to be used, can be performed on the RNA prior to reverse transcription, the cDNA or both. At the time of preparing the cDNA library for a given experiment, all cDNAs in that particular library can be indexed with a six- to eight-base bar code allowing cDNAs from multiple experiments/libraries to be pooled for multiplexed sequencing. Optionally, and particularly where amounts of starting RNA or cDNA are low, the cDNA from a particular preparation can be PCR amplified prior to size selection and final preparation for sequencing. The cDNAs of any given library are then sequenced into a computer-readable format using next generation, high throughput sequencing techniques. There is a number of platforms for such sequencing including those developed by Oxford Nanopore Technologies, Pac Bio. Illumina and others. Illumina's short read sequencing is a commonly used technology for cDNA sequencing and involves the ligation of adaptors to the cDNA, attachment of the DNA to a flow cell and generation of clusters through cycles of bridge amplification and denaturation. Sequencing is then performed through multiple cycles of complementary strand synthesis and laser excitation of bases with reversible terminators.

The depth of sequencing required is dependent on the complexity of the library—the more RNA species there are in the starting sample, the deeper the sequencing required to be able to reliably identify and quantify the rarer RNA species in the sample. The abundance of an RNA in the sample can be determined from the frequency with which this sequence appears in the sequencing readout. Most often this will be compared to the frequency of sequences from RNAs encoding known housekeeping proteins such as beta actin. Where cellular RNA or small RNAs, such as miRNAs, are to be examined, the RNA is often isolated through size selection. Once isolated, linkers can be added to the 3′ and 5′ ends of the RNA, the ligated RNA molecules purified and then cDNA generated through reverse transcription. It will be understood that these technologies are continuing to evolve and improve. For example, to avoid artefacts that might result from ligation, amplification or other sample manipulations, single molecule direct RNA sequencing has been explored by a number of companies including Oxford Nanopore Technologies.

Typically, quantification of biomarkers as performed in the present disclosure will include referenced control samples. In some examples, the control reference is determined from measurements of the biomarkers in corresponding panel of biomarkers from a population of healthy individuals. The term “healthy individual” as used herein refers to a person or populations of persons who are known not to have adenoma, such knowledge being derived from clinical data on the individual which may have been determined from colonoscopy or sigmoidoscopy. In some examples, the control reference is determined from measurements of the corresponding biomarkers in a “typical population”. Preferably, a “typical population” will exhibit a spectrum of adenoma at different stages of disease progression. It is particularly preferred that a “typical population” exhibits the expression characteristics of a cohort of subjects as described herein.

In another example, the control reference may be derived from an established data set including one or more of:
1. a data set comprising measurements of the biomarkers for a population of subjects known to have adenoma;
2. a data set comprising measurements of biomarkers for the subject being tested wherein said measurements have been made previously, such as, for example, when the subject was known to be healthy or, in the case of a subject having adenoma, when the subject was diagnosed or at an earlier stage in disease progression (e.g. benign polyp); and/or
3. a data set comprising measurements of the biomarkers for a healthy individual or a population of healthy individuals.

### Data Analysis

In some examples, methods of determining whether a subject has advanced adenoma or is otherwise at an increased risk of developing advanced adenoma are based upon the biomarker panel measurement compared to a reference profile that can be made in conjunction with statistical analysis.

A quantitative score may be determined by the application of a specific algorithm. The algorithm used to calculate the quantitative score in the methods disclosed herein may group the expression level values of a biomarker or groups of biomarkers. The formation of a particular group of biomarkers, in addition, can facilitate the mathematical weighting of the contribution of various expression levels of biomarker or biomarker subsets (e.g. classifier) to the quantitative score.

In some examples, SPSS software may be used for the statistical analysis. In some examples, binary logistic regression analysis may be used to predict the diagnostic efficiency of the selected biomarkers. In some examples, a statistical algorithm used with a computer to implement the statistical algorithm may be used. In some examples, the statistical algorithm is a learning statistical classifier system. Examples of such systems include Random Forest, interactive tree, classification and regression tree classification or neural networks.

A fair evaluation of a test requires its assessment using “out-of-sample” subjects, that is, subjects not included in the construction of the initial predictive model. This is achieved by assessing the test performance using n-fold cross validation.

Tests for statistical significance include linear and non-linear regression, including ANOVA, Kruskal-Wallis, Wilcoxon, Mann-Whitney and odds ratio, Bayesian probability algorithms. As the number of biomarkers measured increases however, it can be generally more convenient to use a more sophisticated technique such as Random Forests, simple logistic regression, or Bayes Net to name a few.

In some examples, Bayesian probability may be adopted. In this circumstance a 10-fold cross-validation can be used to estimate the “out-of-sample” performance of the models in question. For each combination of biomarkers under consideration, the data can be divided randomly into 10 sub-samples, each with similar proportions of healthy subject and subjects at each stage of disease. In turn, each subsample can be excluded, and a logistic model built using the remaining 90% of the subjects. This model can then be used to estimate the probability of adenoma for the excluded sub-sample, providing an estimate of “out-of-sample” performance. By repeating this for the remaining 9 subsamples, “out-of-sample” performance can be estimated from the study data itself. These “out-of sample” predicted probabilities can then be compared with the actual disease status of the subjects to create a Receiver Operating Characteristic (ROC) Curve, from which the cross-validated sensitivity at a given specificity (e.g. 95% specificity) may be estimated.

Each estimate of “out-of-sample” performance using cross-validation (or any other method), whilst unbiased, has an element of variability to it. Hence a ranking of models (based on biomarker combinations) can be indicative only of the relative performance of such models. However a set of biomarkers which is capable of being used in a large number of combinations to generate a diagnostic test as demonstrated via “out-of-sample” performance evaluations, almost certainly contains within itself combinations of biomarkers that will withstand repeated evaluation.

In one example, a biomarkers are measured using the following algorithm:

$$log ( \frac{p}{1 - p} ) = {β}_{0} + {β}_{BM 1} {C}_{BM 1} + {β}_{BM 2} {C}_{BM 2} + {β}_{BM 3} {C}_{BM 3} + {β}_{BM 4} {C}_{BM 4} + ⋯ \cdot {β}_{BMi} {C}_{BMi}$$

wherein p represents the probability that a person has adenoma. C_{BMi} is the logarithm of concentration of the i^{th} biomarker in the plasma (or serum) of one subject in the cohort being tested. Each beta (β_{BMi}) is a coefficient applying to that biomarker in the concentration units in which it is measured-β₀ is an “offset” or “intercept”. This linear logistic model is common to all results presented herein, but is far from the only way in which a combination of biomarker concentrations may be modelled to predict the probability of adenoma. As would be appreciated by the person skilled in the art, while the base-10 logarithm of the concentration of biomarker is exemplified herein other logarithms can also be used, for example base-2 logarithm. In some examples, the algorithm may include εᵢ which is an error term associated with the model.

It will be understood that, in some examples, one or more demographic or morphometric terms may also be factored into the analysis, for example, using a logistic regression algorithm. The one or more demographic or morphometric terms may be factored into the logistic regression algorithm using any method known to the person skilled in the art. In one example, the one or more demographic or morphometric terms may be assigned an arbitrary value (e.g. 1.0 for males and 1.1 for females or 1.0 for smoker and 1.1. for non-smoker). In one example, the value of the demographic or morphometric term itself will be used in the algorithm (e.g. age in years, BMI). As would be appreciated by the person skilled in the art, BMI or Body Mass Index is a person's weight (for example, in kilograms or pounds) divided by the square of their height (for example, in meters or feet). In one example, the units for BMI is kg/m². In one example, the units for BMI is lb/feet².

In some examples, the methods of the disclosure also contemplate the inclusion of the subject's gender as a biomarker in a biomarker panel described herein. Without wishing to be bound by theory, the subject's gender can be factored into the logistic regression algorithm by assigning an arbitrary value for females and a different arbitrary value for males. As would be understood by the person skilled in the art, the numerical value of the arbitrary value is not important, however it is important that different arbitrary values are assigned for males and females. In one example, the subject's gender can be factored into the logistic regression algorithm by assigning an arbitrary value of 1 for females and 0 for males. In one example, the subject's gender can be factored into the logistic regression algorithm by assigning an arbitrary value of 1.1 for females and 1 for males.

In some examples, the methods of the disclosure also contemplate the inclusion of the subject's age as a biomarker in a biomarker panel described herein. For example, the following algorithm may be used:

$$log ( \frac{p}{1 - p} ) = {β}_{0} + {β}_{BM 1} {C}_{BM 1} + {β}_{BM 2} {C}_{BM 2} + {β}_{BM 3} {C}_{BM 3} + ⋯ \cdot {β}_{BMi} {C}_{BMi} + {β}_{Age} Age$$

wherein the terms are as described herein and Age is the subject's age in years.

Other non-linear or linear logistic algorithms that would be equally applicable include Random Forest, Linear Models for MicroArray data (LIMMA) and/or Significance Analyses of Microarray Data (SAM), Best First, Greedy Stepwise, Naive Bayes, Linear Forward Selection, Scatter Search, Linear Discriminant Analysis (LDA), Stepwise Logistic Regression, Receiver Operating Characteristic and Classification Trees (CT).

The formation of a particular group of biomarkers, in addition, can facilitate the mathematical weighting of the contribution of various expression levels of different biomarker or biomarker subsets (e.g. classifier) to the quantitative score.

The skilled person will be familiar with determination of co-efficient values in regression algorithms. In one example, the coefficients are calculated by the logistic regression software which tests a wide variety of coefficient values for each coefficient to arrive at the one that separates cases and controls with highest sensitivity at a defined specificity. While there is a “best” set of coefficients, there may a very wide variety of values for these coefficients that will give very similar performance that will not be statistically significantly inferior to the “best” one.

The algorithms described herein can be used to derive an adenoma-likelihood score. A score that is above a threshold suggests the subject has a higher likelihood to have APA than someone with a score below the threshold. The score may then inform treatment management.

The skilled person will know that sensitivity refers to the proportion of actual positives in the diagnostic test which are correctly identified as having adenoma. Specificity measures the proportion of negatives which are correctly identified as not having adenoma.

In some examples, the biomarker panel has a sensitivity of at least 5%, at least 10%, at least 15%, at least 20%, at least 25% or at least 30%.

In some examples, the biomarker panel has a specificity of at least 75%, at least 80%, at least 85%, at least 86.4%, at least 90%, or at least 95%.

### Data Handling

It will be apparent from the discussion herein that knowledge-based computer software and hardware for implementing an algorithm also form part of the present disclosure. Such computer software and/or hardware are useful for performing a method of detecting APA according the invention.

The values from the assays described herein can be calculated and stored manually. Alternatively, the statistical analysis steps can be completely or partially performed by a computer program product. The present disclosure thus provides a computer program product including a computer readable storage medium having a computer program stored on it. The program can, when read by a computer, execute relevant calculations based on values obtained from analysis of one or more biological samples from a subject (e.g., gene or protein expression levels, normalization, standardization, thresholding, and conversion of values from assays to a clinical outcome score and/or text or graphical depiction of clinical status or stage and related information). The computer program product has stored therein a computer program for performing the calculation.

The present disclosure also provides systems for executing the data collection and handling or calculating software programs described above, which system generally includes: a) a central computing environment; b) an input device, operatively connected to the computing environment, to receive patient data, wherein the patient data can include, for example, gene or protein expression level or other value obtained from an assay using a biological sample from the patient, or mass spec data or data for any of the assays provided by the present disclosure; c) an output device, connected to the computing environment, to provide information to a user (e.g., medical personnel); and d) an algorithm executed by the central computing environment (e.g., a processor), where the algorithm is executed based on the data received by the input device, and wherein the algorithm calculates an expression score, thresholding, or other functions described herein. The methods provided by the present disclosure may also be automated in whole or in part.

In one example, a method of the disclosure may be used in existing knowledge-based architecture or platforms associated with pathology services. For example, results from a method described herein are transmitted via a communications network (e.g. the internet) to a processing system in which an algorithm is stored and used to generate a predicted posterior probability value which translates to the score of disease probability which is then forwarded to an end user in the form of a diagnostic or predictive report.

The method of the disclosure may, therefore, be in the form of a kit or computer-based system which comprises the reagents necessary to detect the concentration of the biomarkers and the computer hardware and/or software to facilitate determination and transmission of reports to a clinician.

The assays described herein can be integrated into existing or newly developed pathology architecture or platform systems. For example, the present disclosure contemplates a method of allowing a user to determine a subject's risk with respect to adenoma, the method including:
(a) receiving subject data obtained from determining a measurement of each biomarker in a biomarker panel described herein;
(b) processing the data via multivariate analysis to provide a disease score;
(c) determining the status of the subject in accordance with the results of the disease score in comparison with predetermined values; and
(d) transferring an indication of the status of the subject to the user via the communications network reference to the multivariate analysis which includes an algorithm which performs the multivariate analysis function.

The present disclosure also provides software or hardware programmed to implement an algorithm that processes data obtained by performing the method of the disclosure via a multivariate analysis to provide a disease likelihood score and provide or permit a diagnosis or detection of APA in accordance with the results of the disease score in comparison with predetermined values.

### Subjects

Biological samples are collected from subjects who want to determine their likelihood of having a colon polyp or adenoma. The disclosure provides for subjects that can be healthy and asymptomatic. In some examples, the subjects are healthy, asymptomatic and between the ages 20-50. In some examples, the subject is 50 years of age or older. In some examples, the subjects are healthy and asymptomatic and have no family history of adenoma or polyps. In some examples, the subjects are healthy and asymptomatic and have never received a colonoscopy. The disclosure also provides for healthy subjects who are having a test as part of a routine examination, or to establish baseline levels of the biomarkers.

Biological samples may also be collected from subjects who have been determined to have a high risk of colorectal polyps or cancer based on their family history, a who have had previous treatment for colorectal polyps or cancer and/or are in remission. Biological samples may also be collected from subjects who present with physical symptoms known to be associated with colorectal cancer, subjects identified through screening assays (e.g., fecal occult blood testing or sigmoidoscopy) or rectal digital exam or rigid or flexible sigmoidoscopy, colonoscopy or CT scan or CT colonography or other x-ray techniques. Biological samples may also be collected from subjects currently undergoing treatment to determine the effectiveness of therapy or treatment they are receiving.

### Biological Samples

The biomarkers can be measured in different types of biological samples. The sample is preferably from a biological sample that collects and surveys the entire system. Examples of biological sample types useful in this disclosure include one or more of, but are not limited to: urine, stool, whole blood, serum, plasma, blood constituents, lymph fluid, or other fluids produced by the body. In one example, the biological sample is serum. The biomarkers can also be extracted from a biopsy sample, frozen, fixed, paraffin embedded, or fresh.

### Kits

The present invention provides kits for the detection of biomarkers. Such kits may be suitable for detection of nucleic acid species, or alternatively may be for detection of a protein or polypeptide.

For detection of polypeptides, antibodies will most typically be used as components of kits. However, any agent capable of binding specifically to a biomarker gene product will be useful. Other components of the kits will typically include labels, secondary antibodies, inhibitors, co-factors and control gene or protein product preparations to allow the user to quantitate expression levels and/or to assess whether the measurement has worked correctly. Enzyme-linked immunosorbent assay-based (ELISA) tests and competitive ELISA tests are particularly suitable assays that can be carried out easily by the skilled person using kit components.

In some examples, the kit may comprise a substrate, such as a microtitre plate, on which is immobilised capture antibodies corresponding to the biomarkers being measured.

In some examples, the kit comprises beads on which is immobilised capture antibodies corresponding to the biomarkers being measured.

Optionally, the kit further comprises means for the detection of the binding of an antibody to a biomarker polypeptide. Such means include a reporter molecule such as, for example, an enzyme (such as horseradish peroxidase or alkaline phosphatase), a dye, a radionucleotide, a luminescent group, a chemiluminescent group, a fluorescent group, biotin or a colloidal particle, such as colloidal gold or selenium. Preferably such a reporter molecule is directly linked to the antibody.

In one example, a kit may additionally comprise a reference sample. In one embodiment, a reference sample comprises a polypeptide that is detected by an antibody. Preferably, the polypeptide is of known concentration. Such a polypeptide is of particular use as a standard. Accordingly, various known concentrations of such a polypeptide may be detected using a diagnostic assay described herein.

For detection of nucleic acids, such kits may contain a first container such as a vial or plastic tube or a microtiter plate that contains an oligonucleotide probe. The kits may optionally contain a second container that holds primers. The probe may be hybridisable to DNA whose altered expression is associated with colorectal cancer and the primers are useful for amplifying this DNA. Kits that contain an oligonucleotide probe immobilised on a solid support could also be developed, for example, using arrays (see supplement of issue 21(1) Nature Genetics, 1999).

For PCR amplification of nucleic acid, nucleic acid primers may be included in the kit that are complementary to at least a portion of a biomarker gene as described herein. The set of primers typically includes at least two oligonucleotides, preferably four oligonucleotides, that are capable of specific amplification of DNA. Fluorescent-labelled oligonucleotides that will allow quantitative PCR determination may be included (e.g. TaqMan chemistry, Molecular Beacons). Suitable enzymes for amplification of the DNA, will also be included.

Control nucleic acid may also be included for the purposes of comparison or validation. Such controls could either be RNA/DNA isolated from healthy tissue, or from healthy individuals, or housekeeping genes such as β-actin or GAPDH whose mRNA levels are not affected by colorectal cancer.

In other examples, the kit includes blocking, sample dilution and washing solutions. Such buffers are known in the art and are typically optimised for detection and quantification of the biomarkers.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Materials and Methods

### Ethics

All research protocols used in this study were approved by the relevant Human Research Ethics Committees. Written informed consent was obtained from each patient prior to blood sample collection.

### Subject Samples for Examples 1, 2 and 3

Serum samples were taken and processed from a cohort of 53 subjects with a diagnosis of advanced precancerous adenomas (APA) as determined and confirmed by colonoscopy (Males, 57.6%, mean age 65.4 yr, range 38-80 yr; Females 43.4%, mean age 63.8 yr, range 42-79 yr). Subjects having a confirmed diagnosis of both colorectal cancer and APA were not included in the study. Samples were obtained from subjects treated at the Royal Adelaide Hospital (RAH), or the Lyell McEwin Hospital (LMH) in South Australia. Clear FIT results were available for 43 of the APA positive subjects

Subjects who had already received chemotherapy and/or radiotherapy were excluded from the analysis.

The characteristics of the adenomas from these subjects are summarised below in Table 1(a).

Blood was also collected and processed from a group of 143 healthy subjects (Table 1(b)) (64n males (45.1%), mean age 63.2 yr, range 45-85 yr; 79 females (54.9%) mean age 60.0 yr, range 21-84 yr) who had a negative diagnosis for colorectal neoplasia on colonoscopy, had a FIT test and biomarker concentration data for the full set of the 9 blood protein biomarkers being assessed. Of these, 132 had a clear FIT result (Table 1(b)).

TABLE 1(a)
Characteristics of subjects diagnosed with APA who had a FIT test and a complete set of biomarker test results.

| | Subject ID | Gender | Age | FIT result | Diagnosis | Colonoscopy | Adenoma histology |
|---|---|---|---|---|---|---|---|
| 1 | 99952674 | F | 61 | negative | APA | polyps | Tubulovillious Adenoma |
| 2 | 99980087 | M | 66 | negative | APA | polyps | Tubulovillious Adenoma |
| 3 | 99925186 | M | 66 | positive | APA | polyps | Tubulovillious Adenoma |
| 4 | 99011327 | F | 50 | positive | APA | polyps | Tubular Adenoma |
| 5 | 99014493 | M | 68 | negative | APA | Polyps/diverticular | Tubulovillious Adenoma |
| | | | | | | disease | |
| 6 | 90046227 | F | 82 | inconclusive | APA | polyps | ND |
| 7 | 99931549 | F | 51 | negative | APA | polyps | Tubulovillious Adenoma |
| 8 | 90056856 | M | 53 | inconclusive | APA | polyps | Tubular Adenoma/Hyperplastic |
| 9 | 90173605 | M | 66 | Positive | APA | polyps | Tubulovillious Adenoma |
| 10 | 99963399 | M | 69 | Negative | APA | polyps | ND |
| 11 | 12MH0299 | M | 42 | ND | APA | Colonic polyps | Juvenile |
| 12 | 99938062 | M | 79 | negative | APA | polyps | Tubular Adenoma |
| 13 | 90140288 | M | 65 | inconclusive | APA | Polyps | Tubular Adenoma |
| 14 | 12WH0106 | M | 55 | ND | APA | polyps | Tubular adenoma with low grade dysplasia |
| 15 | 99979764 | F | 61 | Negative | APA | polyps | Tubular Adenoma |
| 16 | 90052304 | M | 67 | negative | APA | Polyps | Tubular Adenoma |
| 17 | 99972934 | F | 71 | negative | APA | polyps | Tubulovillious Adenoma |
| 18 | 99935043 | F | 64 | positive | APA | polyps | Tubulovillious Adenoma |
| 19 | 90210046 | M | 38 | negative | APA | polyps | Tubulovillious Adenoma |
| 20 | 90031721 | F | 65 | negative | APA | polyps | Tubulovillous Adenoma |
| 21 | 99983178 | F | 65 | positive | APA | polyps | Tubular Adenoma |
| 22 | 90211628 | F | 67 | negative | APA | polyps | Hyperplastic |
| 23 | 99993220 | M | 80 | negative | APA | Polyps/Diverticula | Tubular Adenoma |
| | | | | | | Disease | |
| 24 | 99938959 | F | 57 | ND | APA | Polyps | Sessile serrated adenoma |
| 25 | 12WH0110 | M | 57 | ND | APA | polyps | Tubular adenoma with low grade dysplasia |
| 26 | 99047282 | F | 68 | negative | APA | polyps | Tubulovillous Adenoma |
| 27 | 99930062 | M | 40 | positive | APA | polyps | Tubulovillous Adenoma |
| 28 | 99937686 | M | 72 | negative | APA | Polyps/Diverticula | Sessile Serrated Adenoma/Hyperplastic |
| | | | | | | Disease | |
| 29 | 99952965 | F | 66 | negative | APA | Polyps | Tubular Adenoma/Tubulovillous Adenoma |
| 30 | 12WH0112 | M | 68 | ND | APA | polyps | ND |
| 31 | 99996942 | M | 72 | Negative | APA | Polyps | Tubulovillous Adenoma |
| 32 | 99924229 | F | 58 | Negative | APA | Polyps | Tubular Adenoma |
| 33 | 99952035 | M | 73 | Negative | APA | Polyps | ND |
| 34 | 90116436 | M | 69 | Negative | APA | Polyps | Tubulovillous Adenoma |
| 35 | 99935561 | M | 63 | Positive | APA | Polyps | Tubulovillous Adenoma |
| 36 | 12WH0046 | M | 69 | ND | APA | Polyps | Tubular adenoma with low grade dysplasia |
| 37 | 99940822 | M | 56 | Positive | APA | Polyps | Tubular Adenoma |
| 38 | 99928033 | F | 78 | Negative | APA | Polyps | Tubulovillous Adenoma |
| 39 | 12WH0115 | M | 74 | ND | APA | Polyps | Tubular adenoma with low grade dysplasia |
| 40 | 99967667 | F | 59 | Negative | APA | Polyps | Tubular Adenoma |
| 41 | 99972634 | F | 79 | Negative | APA | Polyps | Tubulovillous Adenoma |
| 42 | 99935007 | M | 75 | Negative | APA | Polyps | Tubulovillous Adenoma |
| 43 | 99958774 | F | 41 | Negative | APA | Polyps | Hyperplastic |
| 44 | 99961220 | M | 67 | Positive | APA | Polyps | Tubular Adenoma/Tubulovillous Adenoma |
| 45 | 99946921 | M | 75 | Negative | APA | Polyps | Tubular Adenoma |
| 46 | 90202262 | F | 72 | Positive | APA | Polyps | Tubular Adenoma |
| 47 | 99961719 | M | 75 | Negative | APA | Polyps | Tubulovillous Adenoma |
| 48 | 99047230 | M | 75 | Positive | APA | Polyps | Tubulovillous Adenoma |
| 49 | 99934305 | M | 68 | Negative | APA | Polyps | ND |
| 50 | 99952647 | F | 68 | null | APA | Polyps | Tubular Adenoma |
| 51 | 90030477 | F | 71 | Negative | APA | polyps | Tubular Adenoma |
| 52 | 99937433 | F | 50 | Positive | APA | polyps | Tubulovillous Adenoma |
| 53 | 99926853 | F | 64 | Negative | APA | polyps | Sessile Serrated Adenoma |

FIT = fecal immunochemical test
ND = not determined
Tubular adenomas were classified as APA if they were >1cm in the longest dimension

TABLE 1(b)
Characteristics of subjects diagnosed as “Normal” without neoplasia who had a FIT test and a
complete set of biomarker test results.

| | Sample | | | | | Colonoscopy | |
|---|---|---|---|---|---|---|---|
| | ID | Gender | Age | FIT Result | DIAGNOSIS | Findings | Histology |
| 1 | 99943979 | M | 54 | Negative | Negative | Polyps | Hyperplastic |
| 2 | 99929402 | F | 56 | Negative | Negative | Diverticula Disease | |
| 3 | 90119284 | F | 84 | Inconclusive | Negative | Normal | |
| 4 | 90118356 | F | 47 | Negative | Negative | Diverticula Disease | |
| 5 | 99928215 | M | 78 | Positive | Negative | Polyps | Hyperplastic/ |
| | | | | | | | Other = normal |
| | | | | | | | mucosa |
| 6 | 90149369 | F | 62 | Negative | Negative | Normal | |
| 7 | 99961901 | M | 69 | Negative | Negative | Diverticula Disease | |
| 8 | 99932504 | F | 35 | Negative | Negative | Normal | |
| 9 | 99022163 | M | 65 | Negative | Negative | Diverticula Disease | |
| 10 | 99938157 | F | 57 | Negative | Negative | Diverticula Disease | |
| 11 | 99933736 | M | 77 | Negative | Negative | Diverticula Disease | |
| 12 | 90073997 | F | 55 | Inconclusive | Negative | Diverticula Disease | |
| 13 | 99963629 | F | 37 | Negative | Negative | Normal | |
| 14 | 90057329 | F | 57 | Positive | Negative | Diverticula Disease | |
| 15 | 90106589 | F | 63 | Negative | Negative | Normal | |
| 16 | 99926631 | F | 65 | Negative | Negative | Normal | |
| 17 | 99962228 | M | 67 | Negative | Negative | Normal | |
| 18 | 99993093 | M | 83 | Negative | Negative | Diverticula | |
| | | | | | | Disease/Haemorrhoids | |
| 19 | 99939457 | F | 59 | Negative | Negative | Diverticula Disease | |
| 20 | 99936433 | F | 39 | Negative | Negative | Normal | |
| 21 | 90079678 | F | 79 | Inconclusive | Negative | Diverticula Disease | |
| 22 | 99923497 | M | 56 | Negative | Negative | Polyps | Hyperplastic |
| 23 | 99957286 | F | 56 | Negative | Negative | Polyps/Diverticula | Hyperplastic |
| | | | | | | Disease/Haemorrhoids | |
| 24 | 99959517 | M | 68 | Negative | Negative | Normal | |
| 25 | 90104763 | F | 60 | Negative | Negative | Diverticula Disease | |
| 26 | 99939485 | F | 65 | Negative | Negative | Normal | |
| 27 | 99919556 | F | 64 | Negative | Negative | Normal | |
| 28 | 90183306 | F | 57 | Negative | Negative | Polyps | Hyperplastic |
| 29 | 99923767 | F | 40 | Negative | Negative | Normal | |
| 30 | 99977378 | F | 61 | Negative | Negative | Normal/Diverticula | |
| | | | | | | Disease | |
| 31 | 99964296 | F | 66 | Negative | Negative | Normal | |
| 32 | 99940013 | F | 71 | Negative | Negative | Normal | |
| 33 | 99958548 | M | 69 | Negative | Negative | Haemorrhoids | |
| 34 | 99929991 | M | 54 | Negative | Negative | Diverticula Disease | |
| 35 | 90194418 | M | 67 | Negative | Negative | Polyps | Hyperplastic/ |
| | | | | | | | Inflammation |
| 36 | 90141668 | F | 54 | Negative | Negative | Diverticula Disease | |
| 37 | 99991359 | M | 69 | Negative | Negative | Normal | |
| 38 | 99933895 | F | 67 | Negative | Negative | Normal | |
| 39 | 99941591 | M | 85 | Positive | Negative | Diverticula Disease | |
| 40 | 90160185 | M | 45 | Negative | Negative | Normal | |
| 41 | 99919470 | M | 69 | Negative | Negative | Normal | |
| 42 | 90092876 | F | 64 | Negative | Negative | Diverticula Disease | |
| 43 | 99933759 | F | 54 | Negative | Negative | Polyps | Hyperplastic |
| 44 | 99973080 | F | 42 | Negative | Negative | Normal | |
| 45 | 99048761 | F | 64 | Negative | Negative | Diverticula | |
| | | | | | | Disease/Haemorrhoids | |
| 46 | 99957702 | F | 67 | Negative | Negative | Diverticula Disease | |
| 47 | 99942908 | F | 58 | Positive | Negative | Polyps | Hyperplastic |
| 48 | 90198402 | F | 73 | Negative | Negative | Normal | |
| 49 | 99953099 | M | 71 | Negative | Negative | Polyps/Normal mucosa | Other/Normal mucosa |
| 50 | 99959160 | M | 85 | Positive | Negative | Diverticula Disease | |
| 51 | 99972228 | M | 70 | Negative | Negative | Normal | |
| 52 | 90033000 | F | 71 | Negative | Negative | Diverticula Disease | |
| 53 | 99982892 | F | 67 | Negative | Negative | Diverticula Disease | |
| 54 | 99926178 | F | 43 | Negative | Negative | Normal | |
| 55 | 99937019 | F | 70 | Negative | Negative | Normal | |
| 56 | 90203373 | M | 49 | Negative | Negative | Normal | |
| 57 | 99974019 | F | 70 | Negative | Negative | Normal | |
| 58 | 90198305 | M | 46 | Negative | Negative | Polyps | Hyperplastic |
| 59 | 99993212 | M | 60 | Negative | Negative | Polyps | Hyperplastic |
| 60 | 90182929 | F | 71 | Inconclusive | Negative | Normal | |
| 61 | 90030360 | M | 50 | Negative | Negative | Normal | |
| 62 | 90116306 | F | 73 | Negative | Negative | Normal | |
| 63 | 99928824 | M | 72 | Negative | Negative | Normal | |
| 64 | 99937872 | M | 52 | Inconclusive | Negative | Normal | |
| 65 | 99967049 | F | 72 | Negative | Negative | Polyps/Diverticula | Hyperplastic |
| | | | | | | Disease | |
| 66 | 99972858 | F | 70 | Negative | Negative | Diverticula Disease | |
| 67 | 99948803 | F | 48 | Negative | Negative | Normal | |
| 68 | 99978749 | F | 71 | Negative | Negative | Diverticula Disease | |
| 69 | 90056855 | M | 52 | Negative | Negative | Normal | |
| 70 | 90180481 | F | 65 | Positive | Negative | Diverticula Disease | |
| 71 | 99011526 | N | 66 | Inconclusive | Negative | Normal | |
| 72 | 99925544 | M | 75 | Negative | Negative | Normal/Haemorrhoids | |
| 73 | 99947827 | F | 72 | Negative | Negative | Normal | |
| 74 | 90160419 | M | 72 | Negative | Negative | Polyps | Hyperplastic |
| 75 | 99978993 | F | 69 | Negative | Negative | Polyps/Diverticula | Colonic mucosa |
| | | | | | | Disease | showing mild |
| | | | | | | | oedema |
| 76 | 99970694 | F | 72 | Negative | Negative | Polyps/Diverticula | |
| | | | | | | Disease | |
| 77 | 99920398 | F | 70 | Negative | Negative | Haemorrhoids | |
| 78 | 99987812 | F | 49 | Negative | Negative | Normal | |
| 79 | 99958778 | 3 | 79 | Negative | Negative | Normal/Haemorrhoids | |
| 80 | 90149319 | F | 61 | Positive | Negative | Normal | |
| 81 | 99989468 | M | 46 | Negative | Negative | Normal | |
| 82 | 99001168 | F | 61 | Positive | Negative | Haemorrhoids | |
| 83 | 90155049 | M | 56 | Negative | Negative | Normal | |
| 84 | 99962128 | F | 52 | Negative | Negative | Normal | |
| 85 | 99983424 | F | 62 | Negative | Negative | Polyps | Hyperplastic |
| 86 | 99982873 | M | 53 | null | Negative | Normal | |
| 87 | 99045045 | F | 70 | Negative | Negative | Normal | |
| 88 | 90063851 | F | 75 | Inconclusive | Negative | Normal | |
| 89 | 99047225 | F | 54 | Negative | Negative | Normal | |
| 90 | 99978963 | F | 66 | Positive | Negative | Normal | |
| 91 | 90138520 | M | 56 | Negative | Negative | Normal | |
| 92 | 99943638 | F | 77 | Negative | Negative | Polyps | Hyperplastic |
| 93 | 99970782 | F | 53 | Negative | Negative | Normal | |
| 94 | 99936542 | M | 61 | Positive | Negative | Normal | |
| 95 | 99044738 | M | 54 | Negative | Negative | Polyps/Diverticula | Inflammation/ |
| | | | | | | Disease | Other = Normal |
| 96 | 99934313 | M | 56 | Positive | Negative | Polyps | Hyperplastic |
| 97 | 99977065 | F | 64 | Positive | Negative | Diverticula Disease | |
| 98 | 99915136 | M | 45 | Negative | Negative | Diverticula Disease | |
| 99 | 99934037 | 3 | 50 | Negative | Negative | Normal | |
| 100 | 99960843 | F | 72 | Negative | Negative | Diverticula Disease | |
| 101 | 90165092 | F | 43 | Positive | Negative | Normal | |
| 102 | 90221436 | M | 57 | Negative | Negative | Normal | |
| 103 | 90141213 | F | 60 | Inconclusive | Negative | Polyps | Folded colonic |
| | | | | | | | mucosa/without |
| | | | | | | | adenomatous or |
| | | | | | | | hyperplastic change |
| 104 | 99966100 | F | 60 | Negative | Negative | Normal | |
| 105 | 99948830 | M | 73 | Negative | Negative | Polyps/Diverticula | Other/Normal |
| | | | | | | Disease | mucosa |
| 106 | 99930655 | M | 51 | Negative | Negative | Normal/Diverticula | |
| | | | | | | Disease | |
| 107 | 99958642 | M | 51 | Negative | Negative | Normal | |
| 108 | 99000994 | F | 53 | Negative | Negative | Diverticula Disease | |
| 109 | 99941464 | M | 55 | Negative | Negative | Normal/Haemorrhoids | |
| 110 | 99007431 | M | 75 | Negative | Negative | Polyps | Hyperplastic |
| 111 | 99920417 | F | 54 | Negative | Negative | Normal | |
| 112 | 90195874 | M | 66 | Negative | Negative | Polyps/Diverticula | Hyperplastic |
| | | | | | | Disease | |
| 113 | 90199226 | M | 60 | Negative | Negative | Normal | |
| 114 | 99959497 | F | 79 | Positive | Negative | Diverticula Disease | |
| 115 | 90121750 | F | 75 | Negative | Negative | Diverticula Disease | |
| 116 | 99972696 | M | 72 | Positive | Negative | Normal | |
| 117 | 99055170 | F | 43 | Negative | Negative | Normal | |
| 118 | 99940412 | M | 60 | Negative | Negative | Diverticula Disease | |
| 119 | 99965468 | M | 78 | Inconclusive | Negative | Normal | |
| 120 | 99945209 | M | 56 | Negative | Negative | Normal | |
| 121 | 90026924 | M | 65 | Negative | Negative | Normal | |
| 122 | 99977038 | F | 54 | Inconclusive | Negative | Normal/Haemorrhoids | |
| 123 | 99977230 | M | 62 | Negative | Negative | Diverticula Disease | |
| 124 | 90068649 | F | 44 | Negative | Negative | Normal | |
| 125 | 99947991 | F | 56 | Negative | Negative | Normal | |
| 126 | 99947740 | F | 58 | Negative | Negative | Normal | |
| 127 | 90053380 | M | 51 | Negative | Negative | Diverticula Disease | |
| 128 | 90187866 | M | 62 | Negative | Negative | Normal | |
| 129 | 90024022 | M | 68 | Negative | Negative | Normal | |
| 130 | 90220986 | F | 52 | Negative | Negative | Normal | |
| 131 | 99934989 | M | 66 | Negative | Negative | Polyps | Hyperplastic |
| 132 | 90041492 | M | 58 | Positive | Negative | Polyps | Inflammation |
| 133 | 99953348 | M | 63 | Negative | Negative | Diverticula Disease | |
| 134 | 99918599 | F | 21 | Negative | Negative | Normal | |
| 135 | 99935771 | N | 84 | Positive | Negative | Normal | |
| 136 | 90223766 | F | 60 | Negative | Negative | Normal | |
| 137 | 99948949 | M | 64 | Negative | Negative | Diverticula Disease | |
| 138 | 90075570 | M | 67 | Negative | Negative | Diverticula Disease | |
| 139 | 99972259 | F | 60 | Negative | Negative | Normal | |
| 140 | 99952526 | M | 66 | Negative | Negative | Polyps | Hyperplastic |
| 141 | 99941517 | M | 65 | Negative | Negative | Normal | |
| 142 | 99923717 | F | 24 | Negative | Negative | Normal | |
| 143 | 99970494 | F | 75 | Negative | Negative | Diverticula Disease | |

FIT = fecal immunochemical test
ND = not determined
Subjects with Hyperplastic polyps <1 cm in the longest dimension were classified as negative for neoplasia. For full description of colonoscopy findings considered “Negative” for APA (and cancer) see Table 3

### Subject Samples for Example 4

The case/control cohort used for Example 4 is summarized in Table 1(c).

TABLE 1(c)
Cohort details for subjects included
in the APA case/control study

| | Cohort Details | |
|---|---|---|
| | | Advanced Precancerous |
| Characteristics | Control | Adenoma |
| N | 50 | 50 |
| Gender, N | | |
| Female | 28 | 28 |
| Male | 22 | 22 |
| Median age, yrs. | 61 (41-81) | 61 (38-84) |
| (range) | | |

Serum samples were taken and processed from a cohort of 100 subjects. Of these, 50 subjects had a diagnosis of advanced precancerous adenomas (APA) as determined and confirmed by colonoscopy. Subjects having a confirmed diagnosis of both colorectal cancer and APA were not included in the study. In this study, APA included advanced adenomas and includes adenomas of any size displaying high-grade dysplasia or that contain ≥20% villous histologic features. APA also include adenomas (including tubular adenomas and adenomas with low level dysplasia or <20% villous features) or polyps measuring ≥10 mm in the greatest dimension) and sessile serrated polyps measuring 10 mm or more in their longest dimension. Persons simultaneously carrying 3 or more adenomas of any size in their caecum, colon or rectum were also considered as to have APA. Donors of case and control samples used in this study were age and gender matched.

Serum samples used in this study were collected, processed and supplied by the Victorian Cancer Biobank according to their SOP for serum preparation and storage. Samples were freshly frozen and stored at −80° C. prior to use. Research protocols for the study were approved by the Cancer Council Victoria Human Research Ethics Committee (project #HREC 1803).

Concentrations of the five protein biomarkers were quantified in all serum samples derived from patients diagnosed with APA and healthy controls using ELISA kits targeting each individual biomarker, developed by Rhythm Biosciences Limited, Melbourne, Australia.

### Blood Collection and Processing

Serum samples from subjects were collected using a standard operating procedure as previously described (Brierley G V, et al. (2013) Cancer Biomark. 13:67-73). Blood was collected into serum gel tubes (Scientific Specialties Inc., USA) and each sample was left to stand at room temperature for at least 30 min prior to centrifugation (1,200 g, 10 min, room temperature). The serum fraction was then transferred to clean 15 mL tubes and centrifuged again (1,800 g, 10 min, room temperature) prior to being aliquoted (250 μL) and stored (−80° C.). All samples were processed and stored within 2 hrs of collection. Serum samples were only thawed once prior to use.

### Stool Samples

Subjects were also requested to provide a fresh stool sample for faecal immunochemical testing (FOBT). Consenting subjects were provided with a stool sample collection kit and instruction on how to use and return samples for testing.

Briefly, subjects were provided with a Bayer Direct bowel screen kit with instructions for use. For this test, a subject placed a biodegradable cellulose sheet above the water in the toilet bowl and passed a bowel motion. The participant then inserted the tip of a collection probe into the stool and passed it along the stool several times. The probe was then inserted into a collection tube containing storage solution and stored in the fridge. A second sample was collected from a second bowel motion on a subsequent day and both collection tubes were returned to the study site where they were de-identified and sent to a central laboratory for processing (haemoglobin assessment).

### Blood Biomarker Analysis

Sandwich ELISA analysis was used to quantify the levels of nine candidate blood biomarkers in the serum samples provided by volunteers. Details of the biomarkers assessed and the antibodies/ELISA kits used are shown in Table 2. In one example, the antibodies detect the mature polypeptide.

TABLE 2
Sources of antibodies used in the study

| Marker name | UniProtKB | |
|---|---|---|
| and synonyms | No. Protein | Antibody Source |
| DKK-3/REIC | Q9UBP4 | Human Dkk-3 DuoSet ELISA |
| | | Development System (R&D |
| | | DY1118) Head quartered |
| | | Minneapolis USA, sourced through In |
| | | Vitro Technologies, Pty Ltd, Victoria, |
| | | Australia) |
| IGFBP2/IB2/BP2 | P18065 | Human IGFBP-2 ELISA (Demeditec |
| | | DEE005) |
| IL-8/CXCL8 | P10145 | Milliplex MAP Kit High Sensitivity |
| | | Human Cytokine (multiplexing IL8 |
| | | and IL13) |
| | | (Millipore #HSCYTO-60SK) Sourced |
| | | from Merck/Millipore through Thermo |
| | | Fisher Scientific, Scoresby, Victoria, |
| | | Australia |
| IL-13/NC30 | P35225 | Milliplex MAP Kit High Sensitivity |
| | | Human Cytokine (multiplexing IL8 |
| | | and IL13) |
| | | (Millipore #HSCYTO-60SK) Sourced |
| | | from Merck/Millipore through Thermo |
| | | Fisher Scientific, Scoresby, Victoria, |
| | | Australia |
| PKM2/OIP3/ | P14618 | ScheBo Tumor M2-PK ELISA EDTA- |
| PK2/PK3 | | Plasma Test (#08)(ScheBo Biotech |
| | | AG, Giessen, Germany, sourced |
| | | through Abacus dx (9 University |
| | | Drive, Meadowbrook Qld 4131, |
| | | Australia) |
| Mac2BP/ | Q08380 | Human Mac-2BP Platinum ELISA |
| LGALS3BP | | (BMS234) (Bender MedSystems |
| | | GmbH, Austria) |
| TGF 1 beta | P011137 | Human TGF-β1 Quantikine ELISA |
| | | (R&D DB100B) Head quartered |
| | | Minneapolis USA, sourced through In |
| | | Vitro Technologies, Pty Ltd, Victoria, |
| | | Australia) |
| TIMP1/CLG1 | P01033 | Human TIMP-1 Quantikine ELISA |
| | | (R&D DTM100) Head quartered |
| | | Minneapolis USA, sourced through In |
| | | Vitro Technologies, Pty Ltd, Victoria, |
| | | Australia) |
| EpCAM/GA733- | P16422 | DuoSet ELISA kit (R&D Systems, |
| 3/M1S2/M4S1 | | Minneapolis, MN, |
| BDNF | P23560 | Human BDNF Quantikine ELISA |
| | | (R&D DBD00) (R&D Systems, |
| | | Minneapolis USA) |

The human protein sequences are provided appendix 1. The biomarkers may be processed, for example, by removal of a signal sequence, to form a mature polypeptide.

For each assay, samples were measured in duplicate and two in-house quality control (QC) samples were included. One QC sample consisted of pooled serum samples from subjects with diagnosed CRC, the other pooled serum samples from normal control subjects (41 different sera for each pool).

For the standard ELISA, the absorbance or fluorescence signal was detected using the Wallac Victor 1420 multilabel counter (Perkin Elmer, USA). Biomarker concentrations were derived from the respective standard curve using the WorkOut software (Qiagen, Hilden Germany).

### Colonoscopy Assessment

All subjects progressed to colonoscopy as part of their standard of care. Subjects were classified as APA or Negative as described in Table 3.

TABLE 3
Clinical Groups

| | Clinical description at time of |
|---|---|
| Disease group | colonoscopy and by pathology |
| Advanced pre-cancerous | Polyps with: |
| adenomas (APA) | High grade dysplasia (HGD) |
| | Sessile serrated polyps (SSA) with |
| | dysplasia |
| | With ≥20% villous histologic features |
| | Tubulovillous adenoma (TVA) |
| | Villous adenoma (VA) |
| | Any polyp measuring ≥1 cm in the |
| | greatest dimension |
| Negative colonoscopy | True normal (no abnormality) |
| result | Hyperplastic polyp (HPP) |
| | Non advanced adenoma |
| | Diverticular disease |
| | Haemorrhoids |
| | Inflammation |

### Sensitivity and Specificity Determination

The sensitivity of a test (blood biomarker or faecal) for APA is defined as the percentage of colonoscopy-diagnosed cases correctly designated by the test in question.

The specificity of a test for APA is the percentage of colonoscopy-diagnosed disease-free people correctly designated by the test in question. The criteria for diagnosing a subject as APA or Negative are described in Table 3.

To enable a head-to-head comparison between the faecal immunochemical tests (FIT) and blood biomarker panels to accurately detect APA, sensitivity values for all tests (FIT and blood biomarkers considered individually or as panels) were calculated at 86.4% specificity as this was the empirical specificity of FIT when performed in this cohort.

### Empirical Specificity was Calculated as Follows

132 subjects who had a negative diagnosis from colonoscopy also had an interpretable FIT result. Of these, 114 were also negative in FIT.

Therefore, the specificity is determined by the equation:

$$Specificity = \frac{No . Negative by the test}{No . Negative by the test + false positives} \frac{114 \times 100}{132} = 86.4 \%$$

Sensitivity is calculated by

$$Sensitivity = \frac{No . positive by the test}{No . positive by the test + false negatives} \times 100$$

The results for FIT are presented in Table 4 below.

TABLE 4
Specificity and sensitivity of the FIT test for APA

| FIT | No. Correct by test | No. total | Ratio (%) |
|---|---|---|---|
| Specificity | 114 | 132 | 86.4 |
| Sensitivity | 12 | 43 | 27.9 |

Therefore, at a specificity of 86.4% the FIT had a sensitivity for advanced adenomas of only 27.9% as assessed in this cohort. Accordingly, for a blood-based test to be as good as FIT for the detection of APA it should have the same or greater sensitivity at the same specificity for FIT. In other words, the blood-based test should display a sensitivity value greater than 28% at 86.4% specificity.

### Biomarkers Evaluated in the Study

Details of the biomarkers evaluated in the study are provided in Table 2. Specifically, the biomarkers analysed were: insulin-like growth factor binding protein 2 (IGFBP2), dickkoph-related protein 3 (DKK-3), tumour pyruvate kinase isozyme M2 (M2PK), Mac-2 binding protein (Mac2BP), transforming growth factor beta 1 (TGFβ1), tissue inhibitor matrix metalloproteinase 1 (TIMP1), interleukin 8 (IL-8), interleukin 13 (IL-13) and endothelial cell adhesion molecule (EpCAM).

### Blood Biomarker Panel Modelling and Statistical Evaluation

For each of the biomarkers a standard Receiver Operating Characteristic (ROC) analysis was performed by plotting the true positive rate (sensitivity) against the false positive rate (1-specificity) at various threshold settings across the range of concentration values in the full data set for each marker. The sensitivity can then be read off the plot at a threshold value that delivers a specificity value of choice and the standard error determined by a procedure of randomised sampling.

The performance of combinations of biomarkers for the detection of APA was assessed using logistic regression with models being developed that contained one to nine biomarkers based on the equation:

$$Yi = {β}_{0} + {β}_{1} [ {M}_{1} ] + {β}_{2} [ {M}_{2} ] ⋯ \cdot + {ε}_{i}$$

Where:
Yi is a binary indicator of presence or absence of APA, as determined by colonoscopy in the experimental cohort.
β₀ is the regression intercept value.
M1 etc. is the base-10 logarithm of the concentration of biomarker 1, as measured in specified units.
β₁ etc. are the coefficients that are multiplied by the logged biomarker concentration.
ε₁ is an error term associated with the model.

Each individual in the cohort has a diagnosis (APA or normal) determined by colonoscopy (the dependent variable) and their own specific concentrations for each biomarker being considered (the independent variables). Using a statistical software package, a very large range of values for each of the coefficients (β₀-β₉) is tested in combination with each biomarker concentration value (usually the Log of that value) for each biomarker for each subject and the resultant mathematical models most accurately predicting APA/Normal status for the greatest proportion of participants in the cohort are selected. This process is reiterated for each possible biomarker combination for each numerical panel of biomarkers being considered (e.g. 2, 3, 4, - - - , 9 biomarkers). The best candidate biomarker combinations for any given numerical panel and their determined coefficients become the lead algorithms for discriminating APA-derived samples from normal.

To counter problems like overfitting or selection bias often encountered in statistical and machine learning processes, and to give insight into how any given model will generalise to an independent data set, data for each marker were reanalysed using 10-fold cross validation. Briefly, the full data set for any marker was divided into 10 equal sized sub-samples. One sub-sample was retained as a validation data set and the remaining 9 sub-samples were used as training data. This process was repeated 10 times with each of the sub-samples used exactly once as the validation data. In this way, a diagnosis based on biomarker measurements and age, was produced for each sample in the experimental data, without using the measurements colonoscopy-based diagnosis for that sample. Comparison of these diagnoses with the colonoscopy-based diagnoses yields a (10-fold) cross-validated sensitivity estimate.

The sensitivity values in the tables 7-11 below are all tenfold cross validated values along with an associated resampling-based standard error estimate.

The same principle can be applied when additional demographic measures such as age and gender are included in addition to biomarker measurements.

The Prism software package (v6 Graphpad Software Inc., San Diego, CA, USA) and the R statistical software packages were used for statistical analysis. The non-parametric Wilcoxon rank sum test was used to determined statistical difference between cancer and control patients.

### Example 1 Performance of Individual Biomarkers Measured in the Serum of APA and Control Subjects

The clinical characteristics for the subjects analysed in this study are shown in Table 1(a) and (b). A total of 53 subjects with confirmed diagnosis of APA by colonoscopy were analysed. Of the subjects, 49 were 50 or greater years of age. The proportion of males to females was roughly 57% to 43%. 30 of the subjects had been determined to be negative according to FIT.

To enable direct comparison of the performance of FIT and blood biomarkers for the discrimination between APA and Negative, the analysis of blood biomarker sensitivity and specificity was limited to that sub-cohort with both informative FIT and blood biomarker assay results. As the FIT showed an empirical specificity of 86.4% in this cohort, sensitivities for the blood biomarkers, whether assessed individually or in combination, were also determined at specificity of 86.4% using logistic regression.

The median concentrations and range for each biomarker in the case/control data set analysed was determined. IGFBP2, MAC2BP, TGFβ1 and TIMP1 appeared to be expressed more highly in APA subjects than neoplasia-free controls. DKK3 and IL13 were lower in APA cases relative to controls while PKM2, IL8 and EpCAM appeared to be fairly similar in both.

Logistic regression analysis was applied to the raw concentration data to determine the maximum sensitivity achievable with each biomarker at 86.4% specificity. The results are shown in Table 5.

TABLE 5
APA adenoma detection sensitivity for the individual
biomarkers compared to Negative (see Table 3 for definitions)

| | | Sensitivity | Sensitivity at | Sensitivity at |
|---|---|---|---|---|
| | Sensitivity | at 86.4% | 86.4% | 86.4% |
| | at 86.4% | specificity | specificity | specificity |
| | specificity | (10- fold | (Not cross | (10-fold cross |
| | (Not cross | cross | validated) | validated) |
| Biomarker | validated | validated) | with age | with age |
| Mac2BP | 15.1% | 24.53% | 24.5% | 17.0% |
| IGFBP2 | 18.9% | 20.75% | 28.3% | 30.2% |
| IL13 | 19.0% | 18.87% | 26.4% | 18.9% |
| IL8 | 24.5% | 18.87% | 24.5% | 20.8% |
| TIMP1 | 22.6% | 16.98% | 26.4% | 20.8% |
| M2PK | 20.8% | 15.09% | 26.4% | 24.5% |
| DKK3 | 13.2% | 13.21% | 18.9% | 13.2% |
| TGFbeta1 | 11.3% | 13.21% | 26.4% | 17.0% |
| EpCAM | 13.6% | 10.88% | 18.9% | 17.0% |

Considering first the non-cross validated models, inclusion of an additional term for age appears to increase the apparent sensitivity for almost all biomarkers at 86.5% specificity. In the absence of age, IL8 appeared to be the top performing biomarker while when age was included, IGFBP2 produced the highest sensitivity at 86.4% specificity.

Considering the cross validated results, for biomarkers alone, MAC2BP then IGFBP2 followed by IL13 and IL8 showed the highest sensitivities at 86.4% specificity. For biomarkers plus age, IGFBP2 showed the highest cross validated sensitivity at 86.4% specificity followed by PKM2 then TIMP1 and IL8.

Of these single biomarkers, whether considered alone or in combination with age, only IGFBP2 in combination with age, showed a sensitivity at 86.4% specificity that was comparable to or greater than FIT (Table 4).

### Example 2 Identification of Biomarker Panels for APA Detection

In light of the results in Example 1, forward stepwise logistic regressions were performed on biomarker combinations of increasing multiplicity, testing all biomarker combinations of from 2 to 9 markers. The biomarkers examined were IGFBP2, DKK-3, Mac2BP, TGFβ1, TIMP-1, IL-8 IL-13, M2PK, and EpCAM.

The results in Tables 6 to 12 describe combinations of biomarkers only that could detect APA with a sensitivity of greater than 30% at 86.4% specificity, a performance higher than that observed for FIT in these same subjects. The sensitivity values recorded in these tables labelled (a) represent the best values obtained for any given marker combination. High performing marker combinations that also show a 10-fold cross validated Sensitivity >30% at 86.4% specificity are indicated in bold face (described above). Tables labelled (b) show just the ten-fold cross validated combinations with Sensitivities >30% at 86.4% Specificity.

No individual or pairs of biomarkers discriminated APA from Negative samples with a sensitivity exceeding 30% at a specificity of 86.4%,

TABLE 6
Three biomarker combinations having >30% sensitivity at 86.4% specificity.

| | | | Sensitivity | | Sensitivity | | Sensitivity | |
|---|---|---|---|---|---|---|---|---|
| | | | at 86.4% | | at 90% | | at 95% | |
| | | | specificity | | specificity | | specificity | |
| Biomarkers | | | Non-XV | XV | Non-XV | XV | Non-XV | XV |
| IGFBP2 | Mac2BP | TIMP1 | 37.74 | 30.19 | 32.08 | 22.64 | 9.43 | 13.2 |
| IGFBP2 | Mac2BP | TGF beta | 32.08 | 28.3 | 28.3 | 26.42 | 22.64 | 7.55 |

TABLE 7(a)
Four biomarker combinations having >30% sensitivity at 86.4% specificity. Combinations with cross
validated sensitivity >30% at 86.4% specificity are indicated in bold face. Non-XV Sensitivity value not
cross validated, XV - Cross validated sensitivity value.

| | | | | Sensitivity | | Sensitivity | | Sensitivity | |
|---|---|---|---|---|---|---|---|---|---|
| | | | | at 86.4% | | at 90% | | at 95% | |
| | | | | specificity | | specificity | | specificity | |
| Biomarkers | | | | Non-XV | XV | Non-XV | XV | Non-XV | XV |
| IGFBP2 | TIMP1 | IL8 | IL13 | 41.51% | 28.3% | 28.3% | 20.75% | 5.66% | 3.77% |
| IGFBP2 | Mac2BP | TIMP1 | EpCAM | 37.74% | 32.08% | 32.08% | 28.3% | 18.87% | 9.43% |
| IGFBP2 | Mac2BP | TGFbeta | EpCAM | 37.74% | 28.3% | 32.08% | 22.64% | 13.21% | 16.98% |
| IGFBP2 | Mac2BP | TGFbeta | TIMP1 | 37.74% | 28.3% | 28.3% | 22.64% | 15.09% | 3.77% |
| IGFBP2 | Mac2BP | TIMP1 | IL-13 | 35.85% | 32.08% | 28.3% | 22.64% | 15.09% | 3.77% |
| IGFBP2 | DKK3 | Mac2BP | TIMP1 | 35.85% | 26.42% | 32.08% | 18.87% | 18.87% | 13.21% |
| IGFBP2 | DKK3 | Mac2BP | TGFbeta | 33.96% | 26.42% | 28.3% | 26.4% | 18.87% | 11.32% |
| IGFBP2 | TGFbeta | IL8 | EpCAM | 33.96% | 22.64% | 24.53% | 18.87% | 3.77% | 1.89% |
| IGFBP2 | DKK3 | IL8 | IL13 | 33.96% | 22.64% | 20.75% | 15.09% | 5.66% | 5.66% |
| IGFBP2 | IL8 | IL 13 | EpCAM | 33.96% | 20.75% | 20.75% | 16.98% | 7.55% | 5.66% |
| IGFBP2 | Mac2BP | TGFbeta | IL-13 | 32.08% | 30.19% | 30.19% | 24.53% | 18.87% | 9.43% |
| IGFBP2 | TIMP1 | IL13 | EpCAM | 32.08% | 28.3% | 28.3% | 24.53% | 13.21% | 9.43% |
| IGFBP2 | M2PK | Mac2BP | EpCAM | 32.08% | 28.3% | 26.24% | 22.64% | 15.09% | 16.98% |
| IGFBP2 | Mac2BP | TIMP1 | IL13 | 32.08% | 26.42% | 26.42% | 26.42% | 15.09% | 13.21% |
| IGFBP2 | TGFbeta1 | TIMP1 | IL13 | 32.08% | 24.53% | 26.42% | 22.64% | 13.21% | 9.43% |
| IGFBP2 | M2PK | Mac2BP | IL13.S | 32.08% | 24.53% | 26.42% | 18.87% | 16.98% | 11.32% |
| IGFBP2 | TGFbeta1 | IL8 | IL13 | 32.08% | 24.53% | 20.75% | 15.09% | 7.55% | 3.77% |
| IGFBP2 | Dkk3 | TIMP1 | IL13 | 32.08% | 22.64% | 26.42% | 22.64% | 15.09% | 11.32% |
| IGFBP2 | M2PK | Mac2BP | IL8 | 32.08% | 22.64% | 22.64% | 16.98% | 16.98% | 13.21% |
| IGFBP2 | Mac2BP | IL8 | IL13 | 32.08% | 20.75% | 24.53% | 16.98% | 15.09% | 9.43% |
| IGFBP2 | Dkk3 | TIMP1 | EpCAM | 32.08% | 20.75% | 22.64% | 15.09% | 7.55% | 7.55% |
| IGFBP2 | M2PK | IL8 | IL13 | 32.08% | 20.75% | 20.75% | 15.09% | 5.66% | 3.77% |
| IGFBP2 | M2PK | Mac2BP | TGFbeta | 30.19% | 28.30% | 24.53% | 24.53% | 24.53% | 18.87% |
| IGFBP2 | M2PK | Mac2BP | TIMP1 | 30.19% | 26.42% | 28.30% | 26.42% | 16.98% | 11.32% |
| IGFBP2 | Mac2BP | TIMP1 | IL8 | 30.19% | 26.42% | 26.42% | 18.87% | 11.32% | 13.21% |
| IGFBP2 | M2PK | TIMP1 | EpCAM | 30.19% | 26.42% | 22.64% | 16.98% | 9.43% | 7.55% |
| IGFBP2 | Mac2BP | IL13 | EpCAM | 30.19% | 24.53% | 28.30% | 18.87% | 16.98% | 5.66% |
| IGFBP2 | Mac2BP | IL8 | EpCAM. | 30.19% | 24.53% | 26.42% | 18.87% | 13.21% | 9.43% |
| IGFBP2 | Dkk3 | Mac2BP | IL13 | 30.19% | 24.53% | 24.53% | 16.98% | 18.87% | 11.32% |
| IGFBP2 | TIMP1 | IL8 | EpCAM | 30.19% | 24.53% | 22.64% | 13.21% | 9.43% | 9.43% |
| IGFBP2 | Dkk3 | Mac2BP | IL8 | 30.19% | 22.64% | 22.64% | 18.87% | 13.21% | 11.32% |
| IGFBP2 | TGFbeta | TIMP1 | EpCAM | 30.19% | 22.64% | 22.64% | 15.09% | 7.55% | 3.77% |
| IGFBP2 | Dkk3 | IL8 | EpCAM | 30.19% | 20.75% | 18.87% | 15.09% | 3.77% | 1.89% |

TABLE 7(b)
Four biomarker, ten-fold cross validated having >30%
at 86.4% specificity

| | | | | Sensitivity | Sensitivity | Sensitivity |
|---|---|---|---|---|---|---|
| | | | | at 86.4% | at 90% | at 95% |
| | | | | specificity | specificity | specificity |
| | | | | (cross- | (cross | (cross |
| Biomarkers | | | | validated) | validated) | validated) |
| IGFBP2 | Mac2BP | TGFbeta | IL-13 | 30.19 | 24.53 | 9.4 |
| IGFBP2 | Mac2BP | TIMP1 | IL-13 | 32.08 | 22.6 | 3.7 |
| IGFBP2 | Mac2BP | TIMP1 | EpCAM | 32.08 | 28.3 | 9.4 |

TABLE 8(a)
Five biomarker combinations having >30% sensitivity at 86.4% specificity. Combinations with cross validated
sensitivity >30% at 86.4% specificity are indicated in bold face. Non-XV Sensitivity value not cross validated,
XV - Cross validated sensitivity value.

| | | | | | Sensitivity | | Sensitivity | | Sensitivity | |
|---|---|---|---|---|---|---|---|---|---|---|
| | | | | | at 86.4% | | at 90% | | at 95% | |
| | | | | | Specificity | | Specificity | | Specificity | |
| Biomarkers | | | | | Non XV | XV | Non XV | XV | Non XV | XV |
| IGFBP2 | TGFbeta | TIMP1 | IL8 | IL13 | 41.51% | 28.30% | 28.30% | 11.32% | 5.66% | 5.66% |
| IGFBP2 | Dkk3 | TIMP1 | IL8 | IL13 | 41.51% | 26.42% | 28.30% | 13.21% | 7.55% | 1.89% |
| IGFBP2 | TIMP1 | IL8 | IL13 | EpCAM | 39.62% | 28.30% | 28.30% | 20.75% | 5.66% | 1.89% |
| IGFBP2 | Mac2BP | TGFbeta | TIMP1 | EpCAM | 37.74% | 33.96% | 33.96% | 24.53% | 18.87% | 9.43% |
| IGFBP2 | Dkk3 | Mac2BP | TIMP1 | IL13 | 37.74% | 30.19% | 28.30% | 18.87% | 20.75% | 5.66% |
| IGFBP2 | M2PK | Mac2BP | TIMP1 | EpCAM | 37.74% | 28.30% | 28.30% | 24.53% | 22.64% | 15.09% |
| IGFBP2 | Dkk3 | Mac2BP | TIMP1 | EpCAM | 37.74% | 26.42% | 32.08% | 20.75% | 13.21% | 9.43% |
| IGFBP2 | M2PK | Mac2BP | IL8 | IL13 | 37.74% | 22.64% | 26.42% | 18.87% | 11.32% | 7.55% |
| IGFBP2 | M2PK | TIMP1 | IL13 | EpCAM | 35.85% | 32.08% | 28.30% | 26.42% | 11.32% | 9.43% |
| IGFBP2 | Mac2BP | TIMP1 | IL13 | EpCAM | 35.85% | 30.19% | 32.08% | 24.53% | 13.21% | 5.66% |
| IGFBP2 | Mac2BP | TGFbeta | TIMP1 | IL13 | 35.85% | 30.19% | 28.30% | 22.64% | 15.09% | 7.55% |
| IGFBP2 | M2PK | TIMP1 | IL8 | IL13 | 35.85% | 30.19% | 26.42% | 20.75% | 5.66% | 1.89% |
| IGFBP2 | Dkk3 | Mac2BP | TGFbeta | TIMP1 | 35.85% | 26.42% | 32.08% | 24.53% | 18.87% | 9.43% |
| IGFBP2 | Mac2BP | TIMP1 | IL8 | IL13 | 35.85% | 26.42% | 30.19% | 20.75% | 16.98% | 9.43% |
| IGFBP2 | M2PK | Mac2BP | IL8 | EpCAM | 35.85% | 24.53% | 30.19% | 20.75% | 15.09% | 11.32% |
| IGFBP2 | Dkk3 | Mac2BP | TGFbeta | EpCAM | 35.85% | 24.53% | 32.08% | 18.87% | 15.09% | 13.21% |
| IGFBP2 | Dkk3 | Mac2BP | IL8 | EpCAM | 35.85% | 24.53% | 22.64% | 16.98% | 15.09% | 7.55% |
| IGFBP2 | M2PK | TGFbeta | IL8 | IL13 | 35.85% | 24.53% | 24.53% | 16.98% | 9.43% | 3.77% |
| IGFBP2 | Dkk3 | TGFbeta | IL8 | IL13 | 35.85% | 24.53% | 24.53% | 13.21% | 7.55% | 3.77% |
| IGFBP2 | Dkk3 | IL8 | IL13 | EpCAM | 35.85% | 20.75% | 20.75% | 13.21% | 7.55% | 5.66% |
| IGFBP2 | Dkk3 | M2PK | IL8 | IL13 | 35.85% | 18.87% | 20.75% | 15.09% | 5.66% | 3.77% |
| IGFBP2 | M2PK | Mac2BP | TGFbeta | EpCAM | 33.96% | 30.19% | 28.30% | 26.42% | 18.87% | 15.09% |
| IGFBP2 | Dkk3 | M2PK | TIMP1 | IL13 | 33.96% | 28.30% | 28.30% | 22.64% | 16.98% | 11.32% |
| IGFBP2 | Mac2BP | TIMP1 | IL8 | EpCAM | 33.96% | 28.30% | 30.19% | 16.98% | 18.87% | 13.21% |
| IGFBP2 | TGFbeta | TIMP1 | IL13 | EpCAM | 33.96% | 26.42% | 28.30% | 22.64% | 11.32% | 5.66% |
| IGFBP2 | Mac2BP | TGFbeta | IL8 | EpCAM | 33.96% | 24.53% | 22.64% | 18.87% | 13.21% | 11.32% |
| IGFBP2 | Dkk3 | M2PK | Mac2BP | IL8 | 33.96% | 22.64% | 24.53% | 15.09% | 15.09% | 7.55% |
| IGFBP2 | M2PK | IL8 | IL13 | EpCAM | 33.96% | 18.87% | 20.75% | 15.09% | 5.66% | 3.77% |
| IGFBP2 | M2PK | Mac2BP | TIMP1 | IL13. | 32.08% | 28.30% | 32.08% | 26.42% | 18.87% | 16.98% |
| IGFBP2 | M2PK | TGFbeta | TIMP1 | IL 13 | 32.08% | 26.42% | 26.42% | 22.64% | 15.09% | 13.21% |
| IGFBP2 | Mac2BP | TGFbeta | TIMP1 | IL8 | 32.08% | 26.42% | 26.42% | 18.87% | 13.21% | 13.21% |
| IGFBP2 | Dkk3 | TGFbeta | TIMP1 | IL13 | 32.08% | 24.53% | 26.42% | 22.64% | 13.21% | 5.66% |
| IGFBP2 | Dkk3 | M2PK | Mac2BP | TIMP1 | 32.08% | 24.53% | 26.42% | 20.75% | 18.87% | 11.32% |
| IGFBP2 | Dkk3 | TIMP1 | IL13 | EpCAM | 32.08% | 22.64% | 28.30% | 22.64% | 13.21% | 9.43% |
| IGFBP2 | Mac2BP | TGFbeta | IL13 | EpCAM | 32.08% | 22.64% | 30.19% | 20.75% | 11.32% | 7.55% |
| IGFBP2 | M2PK | MAC2BP | IL13.S | EpCAM1 | 32.08% | 22.64% | 22.64% | 18.87% | 16.98% | 11.32% |
| IGFBP2 | MAC2BP | TGFbeta | IL8.S | IL13.S | 32.08% | 22.64% | 26.42% | 15.09% | 13.21% | 7.55% |
| IGFBP2 | M2PK | MAC2BP | TGFbeta | IL13.S | 32.08% | 20.75% | 26.42% | 18.87% | 18.87% | 11.32% |
| IGFBP2 | TGFbeta | IL8.S | IL13.S | EpCAM1 | 32.08% | 20.75% | 22.64% | 15.09% | 7.55% | 3.77% |
| IGFBP2 | Dkk3 | MAC2BP | IL8.S | IL13.S | 32.08% | 20.75% | 26.42% | 15.09% | 9.43% | 7.55% |
| IGFBP2 | Dkk3 | TGFbeta | TIMP1 | EpCAM1 | 32.08% | 18.87% | 22.64% | 15.09% | 7.55% | 3.77% |
| IGFBP2 | Dkk3 | M2PK | MAC2BP | TGFbeta | 30.19% | 28.30% | 24.53% | 22.64% | 18.87% | 15.09% |
| IGFBP2 | M2PK | MAC2BP | TGFbeta | TIMP1 | 30.19% | 26.42% | 28.30% | 26.42% | 16.98% | 11.32% |
| IGFBP2 | Dkk3 | MAC2BP | TGFbeta | IL13.S | 30.19% | 26.42% | 24.53% | 16.98% | 16.98% | 9.43% |
| IGFBP2 | TGFbeta | TIMP1 | IL8.S | EpCAM1 | 30.19% | 26.42% | 26.42% | 13.21% | 9.43% | 1.89% |
| IGFBP2 | Dkk3 | MAC2BP | IL13.S | EpCAM1 | 30.19% | 22.64% | 24.53% | 16.98% | 18.87% | 9.43% |
| IGFBP2 | Dkk3 | MAC2BP | TIMP1 | IL8.S | 30.19% | 20.75% | 28.30% | 18.87% | 15.09% | 11.32% |
| IGFBP2 | M2PK | TGFbeta | IL13.S | EpCAM1 | 30.19% | 18.87% | 24.53% | 16.98% | 11.32% | 9.43% |
| Dkk3 | M2PK | TIMP1 | IL13.S | EpCAM1 | 30.19% | 18.87% | 22.64% | 15.09% | 13.21% | 9.43% |
| IGFBP2 | MAC2BP | IL8.S | IL13.S | EpCAM1 | 30.19% | 16.98% | 24.53% | 13.21% | 11.32% | 9.43% |
| IGFBP2 | Dkk3 | TGFbeta | IL8.S | EpCAM1 | 30.19% | 16.98% | 16.98% | 13.21% | 3.77% | 1.89% |
| IGFBP2 | Dkk3 | TIMP1 | IL8.S | EpCAM1 | 30.19% | 16.98% | 22.64% | 9.43% | 11.32% | 7.55% |

TABLE 8(b)
Five biomarker, ten-fold cross validated combinations having >30% sensitivity at 86.4% specificity

| | | | | | Sensitivity | Sensitivity | Sensitivity |
|---|---|---|---|---|---|---|---|
| | | | | | at 86.4% | at 90% | at 95% |
| | | | | | specificity | specificity | specificity |
| Biomarkers | | | | | (cross-validated) | (cross validated) | (cross validated) |
| IGFBP2 | Mac2BP | TGFbeta | TIMP1 | EpCAM | 34.0 | 24.5 | 9.4 |
| IGFBP2 | M2PK | IL-13 | TIMP1 | EpCAM | 32.08 | 26.4 | 9.4 |
| IGFBP2 | Mac2BP | TGFbeta | M2PK | EpCAM | 30.19 | 26.4 | 15.1 |
| IGFBP2 | Mac2BP | IL-13 | TIMP1 | EpCAM | 30.19 | 24.5 | 5.6 |
| IGFBP2 | Mac2BP | TGFbeta | TIMP1 | IL-13 | 30.19 | 22.6 | 7.5 |
| IGFBP2 | M2PK | IL-13 | TIMP1 | IL-8 | 30.19 | 20.75 | 1.9 |
| IGFBP2 | Mac2BP | IL-13 | TIMP1 | DKK3 | 30.19 | 18.87 | 5.6 |

TABLE 9(a)
Six biomarker, non-cross validated combinations having >30% sensitivity at 86.4% specificity.
Combinations with cross validated sensitivity >30% at 86.4% specificity are indicated in bold face.
Non-XV - Sensitivity value not cross validated, XV - Cross validated sensitivity value.

| | | | | | | Sensitivity | | Sensitivity | | Sensitivity | |
|---|---|---|---|---|---|---|---|---|---|---|---|
| | | | | | | at 86.4% | | at 90% | | at 95% | |
| | | | | | | Specificity | | Specificity | | Specificity | |
| Biomarkers | | | | | | Non-XV | XV | Non-XV | XV | Non-XV | XV |
| IGFBP2 | Dkk3 | TGFbeta | TIMP1 | IL8 | IL13 | 41.51% | 26.42% | 28.30% | 15.09% | 15.09% | 1.89% |
| IGFBP2 | Dkk3 | TIMP1 | IL8 | IL13 | EpCAM | 41.51% | 24.53% | 28.30% | 16.98% | 16.98% | 1.89% |
| IGFBP2 | TGFbeta | TIMP1 | IL8 | IL13 | EpCAM | 39.62% | 28.30% | 28.30% | 16.98% | 16.98% | 3.77% |
| IGFBP2 | M2PK | Mac2BP | IL8 | IL13 | EpCAM | 39.62% | 22.64% | 28.30% | 16.98% | 16.98% | 3.77% |
| IGFBP2 | Dkk3 | Mac2BP | TGFbeta | TIMP1 | IL13 | 37.74% | 32.08% | 28.30% | 16.98% | 16.98% | 5.66% |
| IGFBP2 | M2PK | Mac2BP | TGFbeta | TIMP1 | EpCAM | 37.74% | 28.30% | 28.30% | 24.53% | 24.53% | 11.32% |
| IGFBP2 | Dkk3 | Mac2BP | TGFbeta | TIMP1 | EpCAM | 37.74% | 28.30% | 35.85% | 24.53% | 24.53% | 5.66% |
| IGFBP2 | Mac2BP | TIMP1 | IL8 | IL13 | EpCAM | 37.74% | 26.42% | 32.08% | 18.87% | 18.87% | 5.66% |
| IGFBP2 | Dkk3 | M2PK | Mac2BP | TIMP1 | EpCAM | 37.74% | 24.53% | 28.30% | 20.75% | 20.75% | 11.32% |
| IGFBP2 | Mac2BP | TGFbeta | TIMP1 | IL13 | EpCAM | 35.85% | 28.30% | 33.96% | 22.64% | 22.64% | 3.77% |
| IGFBP2 | M2PK | Mac2BP | TIMP1 | IL8 | IL13 | 35.85% | 28.30% | 32.08% | 20.75% | 20.75% | 9.43% |
| IGFBP2 | Dkk3 | Mac2BP | TIMP1 | IL13 | EpCAM | 35.85% | 26.42% | 32.08% | 20.75% | 20.75% | 5.66% |
| IGFBP2 | M2PK | Mac2BP | TGFbeta | IL8 | IL13 | 35.85% | 24.53% | 26.42% | 20.75% | 20.75% | 7.55% |
| IGFBP2 | M2PK | TGFbeta | IL8 | IL13 | EpCAM | 35.85% | 24.53% | 24.53% | 18.87% | 18.87% | 3.77% |
| IGFBP2 | Dkk3 | Mac2BP | TIMP1 | IL8 | IL13 | 35.85% | 24.53% | 30.19% | 16.98% | 16.98% | 3.77% |
| IGFBP2 | Dkk3 | M2PK | TGFbeta | IL8 | IL13 | 35.85% | 24.53% | 24.53% | 16.98% | 16.98% | 3.77% |
| IGFBP2 | Dkk3 | Mac2BP | TGFbeta | IL8 | EpCAM | 35.85% | 24.53% | 24.53% | 15.09% | 15.09% | 9.43% |
| IGFBP2 | M2PK | Mac2BP | TIMP1 | IL13 | EpCAM | 33.96% | 30.19% | 30.19% | 24.53% | 24.53% | 16.98% |
| IGFBP2 | M2PK | TIMP1 | IL8 | IL13 | EpCAM | 33.96% | 28.30% | 30.19% | 22.64% | 22.64% | 1.89% |
| IGFBP2 | Dkk3 | M2PK | Mac2BP | TGFbeta | EpCAM | 33.96% | 28.30% | 26.42% | 22.64% | 22.64% | 11.32% |
| IGFBP2 | Mac2BP | TGFbeta | TIMP1 | IL8 | EpCAM | 33.96% | 28.30% | 30.19% | 20.75% | 20.75% | 11.32% |
| IGFBP2 | Mac2BP | TGFbeta | TIMP1 | IL8 | IL13 | 33.96% | 26.42% | 30.19% | 20.75% | 20.75% | 9.43% |
| IGFBP2 | Dkk3 | M2PK | TIMP1 | IL8 | IL13 | 33.96% | 26.42% | 26.42% | 20.75% | 20.75% | 1.89% |
| IGFBP2 | M2PK | Mac2BP | TGFbeta | IL8 | EpCAM | 33.96% | 26.42% | 28.30% | 16.98% | 16.98% | 13.21% |
| IGFBP2 | M2PK | TGFbeta | TIMP1 | IL8 | IL13 | 33.96% | 26.42% | 28.30% | 16.98% | 16.98% | 1.89% |
| IGFBP2 | Dkk3 | Mac2BP | TIMP1 | IL8 | EpCAM | 33.96% | 24.53% | 30.19% | 18.87% | 18.87% | 11.32% |
| IGFBP2 | Dkk3 | TGFbeta | TIMP1 | IL13 | EpCAM | 33.96% | 22.64% | 28.30% | 20.75% | 20.75% | 7.55% |
| IGFBP2 | Dkk3 | M2PK | Mac2BP | IL8 | EpCAM | 33.96% | 22.64% | 30.19% | 13.21% | 13.21% | 9.43% |
| IGFBP2 | Dkk3 | M2PK | Mac2BP | IL8 | IL13 | 33.96% | 20.75% | 26.42% | 15.09% | 15.09% | 5.66% |
| IGFBP2 | Dkk3 | M2PK | Mac2BP | TIMP1 | IL13 | 32.08% | 30.19% | 32.08% | 22.64% | 22.64% | 13.21% |
| IGFBP2 | M2PK | TGFbeta | TIMP1 | IL13 | EpCAM | 32.08% | 28.30% | 30.19% | 22.64% | 22.64% | 11.32% |
| IGFBP2 | Dkk3 | M2PK | TIMP1 | IL13 | EpCAM | 32.08% | 28.30% | 30.19% | 22.64% | 22.64% | 7.55% |
| IGFBP2 | M2PK | Mac2BP | TGFbeta | TIMP1 | IL13 | 32.08% | 26.42% | 32.08% | 24.53% | 24.53% | 11.32% |
| IGFBP2 | Dkk3 | M2PK | TGFbeta | TIMP1 | IL13 | 32.08% | 26.42% | 26.42% | 20.75% | 20.75% | 9.43% |
| IGFBP2 | Dkk3 | M2PK | Mac2BP | TGFbeta | TIMP1 | 32.08% | 24.53% | 26.42% | 22.64% | 22.64% | 13.21% |
| IGFBP2 | Dkk3 | Mac2BP | TGFbeta | IL13 | EpCAM | 32.08% | 24.53% | 28.30% | 20.75% | 20.75% | 7.55% |
| IGFBP2 | Dkk3 | TGFbeta | IL8 | IL13 | EpCAM | 32.08% | 22.64% | 24.53% | 15.09% | 15.09% | 3.77% |
| IGFBP2 | Dkk3 | Mac2BP | TGFbeta | IL8 | IL13 | 32.08% | 22.64% | 30.19% | 15.09% | 15.09% | 7.55% |
| IGFBP2 | Dkk3 | M2PK | Mac2BP | TGFbeta | IL8 | 32.08% | 20.75% | 22.64% | 16.98% | 16.98% | 11.32% |
| IGFBP2 | Mac2BP | TGFbeta | IL8 | IL13 | EpCAM | 32.08% | 20.75% | 26.42% | 11.32% | 11.32% | 7.55% |
| IGFBP2 | Dkk3 | Mac2BP | IL8 | IL13 | EpCAM | 32.08% | 18.87% | 26.42% | 15.09% | 15.09% | 5.66% |
| IGFBP2 | Dkk3 | M2PK | IL8 | IL13 | EpCAM | 32.08% | 16.98% | 18.87% | 15.09% | 15.09% | 3.77% |
| IGFBP2 | M2PK | Mac2BP | TIMP1 | IL8 | EpCAM | 30.19% | 26.42% | 28.30% | 22.64% | 22.64% | 11.32% |
| IGFBP2 | Dkk3 | Mac2BP | TGFbeta | TIMP1 | IL8 | 30.19% | 22.64% | 26.42% | 18.87% | 18.87% | 13.21% |
| IGFBP2 | Dkk3 | M2PK | Mac2BP | TGFbeta | IL13 | 30.19% | 22.64% | 24.53% | 16.98% | 16.98% | 11.32% |
| IGFBP2 | M2PK | Mac2BP | TGFbeta | IL13 | EpCAM | 30.19% | 20.75% | 28.30% | 18.87% | 18.87% | 9.43% |
| IGFBP2 | Dkk3 | M2PK | Mac2BP | IL13 | EpCAM | 30.19% | 20.75% | 24.53% | 18.87% | 18.87% | 9.43% |
| IGFBP2 | Dkk3 | M2PK | TGFbeta | IL13 | EpCAM | 30.19% | 16.98% | 22.64% | 16.98% | 16.98% | 5.66% |
| IGFBP2 | Dkk3 | M2PK | TGFbeta | TIMP1 | IL8 | 30.19% | 16.98% | 18.87% | 15.09% | 15.09% | 7.55% |
| Dkk3 | M2PK | Mac2BP | TIMP1 | IL13 | EpCAM | 30.19% | 16.98% | 22.64% | 13.21% | 13.21% | 7.55% |
| IGFBP2 | Dkk3 | TGFbeta | TIMP1 | IL8 | EpCAM | 30.19% | 15.09% | 20.75% | 9.43% | 9.43% | 1.89% |
| Dkk3 | M2PK | Mac2BP | TGFbeta | IL8 | IL13 | 30.19% | 15.09% | 18.87% | 9.43% | 9.43% | 7.55% |
| Dkk3 | M2PK | Mac2BP | IL8 | IL13 | EpCAM | 30.19% | 13.21% | 20.75% | 13.21% | 13.21% | 3.77% |

TABLE 9(b)
Six biomarker, ten-fold Cross validated combinations having >30% sensitivity at 86.4% specificity.

| | | | | | | Sensitivity | Sensitivity | Sensitivity |
|---|---|---|---|---|---|---|---|---|
| | | | | | | at 86.4% | at 90% | at 95% |
| | | | | | | specificity | specificity | specificity |
| Biomarkers | | | | | | (cross-validated) | (cross validated) | (cross validated) |
| IGFBP2 | Mac2BP | TGFbeta | TIMP1 | DKK3 | IL-13 | 32.08 | 17.0 | 5.7 |
| IGFBP2 | Mac2BP | M2PK | TIMP1 | EpCAM | IL-13 | 30.19 | 24.5 | 16.9 |
| IGFBP2 | Mac2BP | M2PK | TIMP1 | DKK3 | IL-13 | 30.19 | 22.6 | 13.2 |

TABLE 10
Seven biomarker combinations having >30% sensitivity at 86.4% specificity.
No Combinations cross validated with sensitivity >30% at 86.4% specificity.
Non-XV - Sensitivity value not cross validated, XV - Cross validated sensitivity value.

| | | | | | | | Sensitivity | | Sensitivity | | Sensitivity | |
|---|---|---|---|---|---|---|---|---|---|---|---|---|
| | | | | | | | at 86.4% | | at 90% | | at 95% | |
| | | | | | | | Specificity | | Specificity | | Specificity | |
| Biomarkers | | | | | | | Non XV | XV | Non XV | XV | Non XV | XV |
| IGFBP2 | Dkk3 | TGFbeta | TIMP1 | IL8 | IL13 | EpCAM | 41.51% | 24.53% | 28.30% | 15.09% | 5.66% | 1.89% |
| IGFBP2 | Dkk3 | M2PK | Mac2BP | TGFbeta | TIMP1 | EpCAM | 37.74% | 22.64% | 28.30% | 20.75% | 20.75% | 9.43% |
| IGFBP2 | Dkk3 | Mac2BP | TIMP1 | IL8 | IL13 | EpCAM | 37.74% | 22.64% | 32.08% | 16.98% | 13.21% | 3.77% |
| IGFBP2 | Dkk3 | Mac2BP | TGFbeta | TIMP1 | IL13 | EpCAM | 35.85% | 26.42% | 33.96% | 20.75% | 15.09% | 5.66% |
| IGFBP2 | Mac2BP | TGFbeta | TIMP1 | IL8 | IL13 | EpCAM | 35.85% | 26.42% | 32.08% | 16.98% | 13.21% | 7.55% |
| IGFBP2 | Dkk3 | Mac2BP | TGFbeta | TIMP1 | IL8 | IL13 | 35.85% | 26.42% | 32.08% | 16.98% | 16.98% | 3.77% |
| IGFBP2 | Dkk3 | M2PK | Mac2BP | TIMP1 | IL8 | IL13 | 35.85% | 26.42% | 32.08% | 16.98% | 16.98% | 5.66% |
| IGFBP2 | Dkk3 | M2PK | TIMP1 | IL8 | IL13 | EpCAM | 35.85% | 24.53% | 26.42% | 20.75% | 7.55% | 1.89% |
| IGFBP2 | M2PK | Mac2BP | TGFbeta | TIMP1 | IL8 | IL13 | 35.85% | 24.53% | 32.08% | 20.75% | 18.87% | 9.43% |
| IGFBP2 | Dkk3 | M2PK | TGFbeta | TIMP1 | IL8 | IL13 | 35.85% | 24.53% | 28.30% | 18.87% | 9.43% | 1.89% |
| IGFBP2 | Dkk3 | M2PK | Mac2BP | TGFbeta | IL8 | IL13 | 35.85% | 22.64% | 28.30% | 18.87% | 15.09% | 3.77% |
| IGFBP2 | M2PK | Mac2BP | TGFbeta | IL8 | IL13 | EpCAM | 35.85% | 22.64% | 28.30% | 16.98% | 11.32% | 5.66% |
| IGFBP2 | Dkk3 | M2PK | TGFbeta | IL8 | IL13 | EpCAM | 35.85% | 20.75% | 24.53% | 16.98% | 5.66% | 3.77% |
| IGFBP2 | M2PK | Mac2BP | TGFbeta | TIMP1 | IL13 | EpCAM | 33.96% | 28.30% | 32.08% | 22.64% | 15.09% | 15.09% |
| IGFBP2 | Dkk3 | M2PK | Mac2BP | TIMP1 | IL13 | EpCAM | 33.96% | 26.42% | 32.08% | 20.75% | 18.87% | 11.32% |
| IGFBP2 | Dkk3 | Mac2BP | TGFbeta | TIMP1 | IL8 | EpCAM | 33.96% | 26.42% | 32.08% | 20.75% | 18.87% | 9.43% |
| IGFBP2 | M2PK | TGFbeta | TIMP1 | IL8 | IL13 | EpCAM | 33.96% | 26.42% | 30.19% | 18.87% | 3.77% | 1.89% |
| IGFBP2 | M2PK | Mac2BP | TIMP1 | IL8 | IL13 | EpCAM | 33.96% | 26.42% | 33.96% | 18.87% | 15.09% | 5.66% |
| IGFBP2 | Dkk3 | M2PK | TGFbeta | TIMP1 | IL13 | EpCAM | 33.96% | 22.64% | 28.30% | 20.75% | 9.43% | 9.43% |
| IGFBP2 | Dkk3 | M2PK | Mac2BP | IL8 | IL13 | EpCAM | 33.96% | 20.75% | 26.42% | 13.21% | 13.21% | 3.77% |
| IGFBP2 | M2PK | Mac2BP | TGFbeta | TIMP1 | IL8 | EpCAM | 32.08% | 26.42% | 28.30% | 22.64% | 18.87% | 11.32% |
| IGFBP2 | Dkk3 | M2PK | Mac2BP | TGFbeta | TIMP1 | IL13 | 32.08% | 26.42% | 32.08% | 18.87% | 18.87% | 9.43% |
| IGFBP2 | Dkk3 | M2PK | Mac2BP | TGFbeta | IL13 | EpCAM | 32.08% | 18.87% | 26.42% | 18.87% | 16.98% | 11.32% |
| IGFBP2 | Dkk3 | M2PK | Mac2BP | TGFbeta | IL8 | EpCAM | 32.08% | 18.87% | 28.30% | 15.09% | 15.09% | 9.43% |
| IGFBP2 | Dkk3 | Mac2BP | TGFbeta | IL8 | IL13 | EpCAM | 32.08% | 18.87% | 28.30% | 11.32% | 9.43% | 5.66% |
| Dkk3 | M2PK | Mac2BP | TGFbeta | IL8 | IL13 | EpCAM | 32.08% | 13.21% | 16.98% | 7.55% | 13.21% | 3.77% |
| IGFBP2 | Dkk3 | M2PK | Mac2BP | TIMP1 | IL8 | EpCAM | 30.19% | 22.64% | 28.30% | 15.09% | 18.87% | 9.43% |

TABLE 11
Eight biomarker combinations having >30% sensitivity at 86.4% specificity.
No Combinations cross validated with sensitivity >30% at 86.4% specificity.
Non-XV - Sensitivity value not cross validated, XV - Cross validated sensitivity value.

| | | | | | | | | Sensitivity | | Sensitivity | | Sensitivity | |
|---|---|---|---|---|---|---|---|---|---|---|---|---|---|
| | | | | | | | | at 86.4% | | at 90% | | at 95% | |
| | | | | | | | | Specificity | | Specificity | | Specificity | |
| Biomarkers | | | | | | | | Non XV | XV | Non XV | XV | Non XV | XV |
| IGFBP2 | Dkk3 | M2PK | Mac2BP | TGFbeta | IL8 | IL13 | EpCAM | 37.74% | 18.87% | 28.30% | 15.09% | 11.32% | 3.77% |
| IGFBP2 | M2PK | Mac2BP | TGFbeta | TIMP1 | IL8 | IL13 | EpCAM | 35.85% | 26.42% | 32.08% | 18.87% | 18.87% | 5.66% |
| IGFBP2 | Dkk3 | Mac2BP | TGFbeta | TIMP1 | IL8 | IL13 | EpCAM | 35.85% | 24.53% | 32.08% | 16.98% | 13.21% | 3.77% |
| IGFBP2 | Dkk3 | M2PK | TGFbeta | TIMP1 | IL8 | IL13 | EpCAM | 35.85% | 24.53% | 26.42% | 16.98% | 9.43% | 1.89% |
| IGFBP2 | Dkk3 | M2PK | Mac2BP | TGFbeta | TIMP1 | IL8 | IL13 | 35.85% | 24.53% | 32.08% | 16.98% | 20.75% | 3.77% |
| IGFBP2 | Dkk3 | M2PK | Mac2BP | TGFbeta | TIMP1 | IL13 | EpCAM | 33.96% | 26.42% | 32.08% | 24.53% | 16.98% | 9.43% |
| IGFBP2 | Dkk3 | M2PK | Mac2BP | TIMP1 | IL8 | IL13 | EpCAM | 33.96% | 24.53% | 33.96% | 18.87% | 16.98% | 3.77% |
| IGFBP2 | Dkk3 | M2PK | Mac2BP | TGFbeta | TIMP1 | IL8 | EpCAM | 30.19% | 24.53% | 28.30% | 16.98% | 18.87% | 9.43% |

TABLE 12
Nine biomarker combination having >30% sensitivity at 86.4% specificity.
No Combinations cross validated with sensitivity >30% at 86.4% specificity.
Non-XV - Sensitivity value not cross validated, XV - Cross validated sensitivity value.

| | | | | | | | | | Sensitivity | | Sensitivity | | Sensitivity | |
|---|---|---|---|---|---|---|---|---|---|---|---|---|---|---|
| | | | | | | | | | at 86.4% | | at 90% | | at 95% | |
| | | | | | | | | | Specificity | | Specificity | | Specificity | |
| Biomarkers | | | | | | | | | Non XV | XV | Non XV | XV | Non XV | XV |
| IGFBP2 | Dkk3 | M2PK | Mac2BP | TGFbeta | TIMP1 | IL8 | IL13 | EpCAM | 35.85% | 24.53% | 32.08% | 18.87% | 20.75% | 3.77% |

Although models for 7, 8 and 9, biomarker panels could be identified that produced non-cross validated sensitivities of >30% at 86.4% specificity as indicated in tables 10-12, none of these cross validated with a sensitivity of >30% at 86.4% specificity.

### Example 3 the Impact of Coupling Demographic and Biomarker Data on APA Detection

The risk of developing adenomas and colorectal cancer is impacted by a number of demographic, nutritional and lifestyle factors. Age is a key factor impacting colorectal cancer risk with the incidence of this disease rising dramatically above the age of 50 years. Other factors shown to increase colorectal cancer risk include being overweight or obese, tall, physically inactive and consuming processed meats (16% per 50 g per day), red meat (12% per 100 g per day, colon cancer only) and alcohol above 30 g/day (non-linear, 15% for 30 g per day; 25% for 40 g per day) and smoking tobacco. Also, males are more likely to develop colorectal cancer than females (World Cancer Research Fund/American Institute for Cancer Research. Continuous Updater Project Expert Panel Report 2018. Diet, Nitration, physical activity and colorectal cancer. Available at dietandcancerreport.org).

As over 90% of colorectal cancers have their origins in adenomas these factors are also expected to increase the risk of developing APA. Age was therefore included as a variable along with biomarkers, considered either singly or in combination and the impact on APA detection examined as for Example 2.

The results in Tables 13 to 20 describe combinations of biomarkers, with the addition of age, that could detect APA with a sensitivity of greater than 30% at 86.4% specificity, a performance higher than that observed for FIT in these same subjects. In tables labelled (a), biomarker combinations (plus age) are ranked from top to bottom based on their non-cross validated Sensitivity value determined at 86.4% Specificity. Corresponding cross validated Sensitivity values for these top performing combinations are also shown. Where the cross validated sensitivity for a combination also exceeds 30% at 86.4% specificity, it has been indicated in boldface. Tables labelled (b) show data only for those biomarker combinations (plus age) producing ten-fold cross validated sensitivities >30% at 86.4% specificity. (Note that high performing cross validated combinations that have a corresponding non-cross validated sensitivity of <30% at 86.4% specificity will not be represented in the relevant table (a)).

One single biomarker, IGFBP2, when modelled in combination with age, showed a ten-fold cross-validated sensitivity for differentiation between APA and Negative of 30.19% at 86.4% specificity (corresponding non-cross validated sensitivity, 28.3%).

TABLE 13
Two biomarker cross validated combinations plus age
having >30% sensitivity at 86.4% specificity.
Combinations also showing a cross validated sensitivity
>30% at 86.4% specificity are indicated in bold face.

| | | Sensitivity | Sensitivity | Sensitivity | Sensitivity |
|---|---|---|---|---|---|
| | | at 86.4% | at 86.4% | at 90% | at 95% |
| | | Specificity | specificity | specificity | specificity |
| | | (non cross- | (cross- | (cross- | (cross- |
| Biomarkers | | validated) | validated) | validated) | validated) |
| IGFBP2 | Mac2BP | 32.08 | 32.08 | 26.42 | 15.1 |
| IGFBP2 | TGFbeta | 33.96 | 32.08 | 18.87 | 5.6 |
| IGFBP2 | TIMP1 | 33.96 | 30.19 | 13.21 | 7.5 |
| IGFBP2 | EpCAM | 30.19 | 30.19 | 26.4 | 9.4 |
| IGFBP2 | DKK-3 | 30.19 | 28.30 | 24.53 | 7.55 |
| IGFBP2 | M2PK | 32.08 | 26.42 | 18.87 | 11.32 |

TABLE 14(a)
Three biomarker, non-cross validated combinations plus age having >30% sensitivity at 86.4% specificity.
Combinations also showing a cross validated sensitivity >30% at 86.4% specificity are indicated in bold face.
Non-XV - Sensitivity value not cross validated, XV - Cross validated sensitivity value.

| | | | Sensitivity | | Sensitivity | | Sensitivity | |
|---|---|---|---|---|---|---|---|---|
| | | | at 86.4% | | at 90% | | at 95% | |
| | | | Specificity | | Specificity | | Specificity | |
| Biomarker | | | Non XV | XV | Non XV | XV | Non XV | XV |
| IGFBP2 | Mac2BP | TGFbeta1 | 35.85% | 32.08% | 30.19% | 30.19% | 22.64% | 11.32% |
| IGFBP2 | Mac2BP | TIMP1 | 35.85% | 32.08% | 32.08% | 24.53% | 9.43% | 7.55% |
| IGFBP2 | IL8 | IL13 | 35.85% | 24.53% | 26.42% | 15.09% | 7.55% | 3.77% |
| IGFBP2 | TGFbeta1 | TIMP1 | 35.85% | 30.19% | 26.42% | 15.09% | 11.32% | 7.55% |
| IGFBP2 | TIMP1 | IL8 | 33.96% | 22.64% | 20.75% | 20.75% | 7.55% | 5.66% |
| IGFBP2 | TIMP1 | EpCAM | 32.08% | 26.42% | 28.30% | 24.53% | 7.55% | 7.55% |
| IGFBP2 | M2PK | TGFbeta1 | 32.08% | 28.30% | 30.19% | 24.53% | 20.75% | 9.43% |
| IGFBP2 | M2PK | Mac2BP | 32.08% | 28.30% | 24.53% | 24.53% | 20.75% | 13.21% |
| IGFBP2 | M2PK | EpCAM | 32.08% | 24.53% | 24.53% | 20.75% | 11.32% | 9.43% |
| IGFBP2 | Dkk3 | TGFbeta1 | 32.08% | 28.30% | 28.30% | 20.75% | 15.09% | 9.43% |
| IGFBP2 | IL13 | EpCAM | 32.08% | 28.30% | 26.42% | 18.87% | 9.43% | 3.77% |
| IGFBP2 | Mac2BP | IL13.S | 32.08% | 22.64% | 26.42% | 18.87% | 18.87% | 11.32% |
| IGFBP2 | M2PK | TIMP1 | 32.08% | 22.64% | 30.19% | 15.09% | 16.98% | 9.43% |
| IGFBP2 | Mac2BP | EpCAM | 30.19% | 28.30% | 24.53% | 26.42% | 18.87% | 9.43% |
| IGFBP2 | IL8 | EpCAM | 30.19% | 24.53% | 22.64% | 22.64% | 7.55% | 3.77% |
| IGFBP2 | TIMP1 | IL13 | 30.19% | 22.64% | 28.30% | 22.64% | 13.21% | 5.66% |
| IGFBP2 | Mac2BP | IL8 | 30.19% | 24.53% | 24.53% | 20.75% | 16.98% | 15.09% |
| IGFBP2 | TGFbeta1 | IL13 | 30.19% | 24.53% | 26.42% | 18.87% | 16.98% | 11.32% |
| IGFBP2 | Dkk3 | IL13 | 30.19% | 28.30% | 24.53% | 18.87% | 13.21% | 3.77% |
| TIMP1 | IL8 | IL13 | 30.19% | 18.87% | 16.98% | 15.09% | 11.32% | 7.55% |
| IGFBP2 | Dkk3 | M2PK | 30.19% | 24.53% | 20.75% | 15.09% | 13.21% | 7.55% |
| IGFBP2 | M2PK | IL13 | 30.19% | 24.53% | 18.87% | 13.21% | 13.21% | 11.32% |
| IGFBP2 | Dkk3 | TIMP1 | 30.19% | 24.53% | 22.64% | 13.21% | 16.98% | 7.55% |
| Dkk3 | Mac2BP | IL8 | 30.19% | 11.32% | 20.75% | 9.43% | 7.55% | 3.77% |

TABLE 14(b)
Three biomarker, ten-fold cross validated combinations plus
age having >30% sensitivity at 86.4% specificity.

| | | | Sensitivity | Sensitivity | Sensitivity |
|---|---|---|---|---|---|
| | | | at 86.4% | at 90% | at 95% |
| | | | specificity | specificity | specificity |
| | | | (cross- | (cross- | (cross- |
| Biomarkers | | | validated | validated) | validated) |
| IGFBP2 | Mac2BP | TIMP1 | 32.08 | 24.53 | 7.5 |
| IGFBP2 | Mac2BP | TGFbeta | 32.08 | 30.19 | 11.3 |
| IGFBP2 | Mac2BP | DKK3 | 30.19 | 24.5 | 11.3 |
| IGFBP2 | TGFbeta | TIMP1 | 30.19 | 15.1 | 7.5 |
| IGFBP2 | TGFbeta | EpCAM | 30.19 | 26.4 | 7.6 |

TABLE 15(a)
Four biomarker, non-cross validated combinations plus age having >30% sensitivity at 86.4% specificity.
Combinations also showing a cross validated sensitivity >30% at 86.4% specificity are indicated in bold face.
Non-XV - Sensitivity value not cross validated, XV - Cross validated sensitivity value.

| | | | | Sensitivity | | Sensitivity | | Sensitivity | |
|---|---|---|---|---|---|---|---|---|---|
| | | | | at 86.4% | | at 90% | | at 95% | |
| | | | | Specificity | | Specificity | | Specificity | |
| Biomarker | | | | Non XV | XV | Non XV | XV | Non XV | XV |
| IGFBP2 | Mac2BP | TGFbeta | TIMP1 | 39.62% | 32.08% | 32.08% | 28.30% | 11.32% | 9.43% |
| IGFBP2 | Mac2BP | TIMP1 | EpCAM | 39.62% | 32.08% | 30.19% | 24.53% | 20.75% | 9.43% |
| IGFBP2 | TIMP1 | IL8 | IL13 | 39.62% | 28.30% | 26.42% | 22.64% | 3.77% | 1.89% |
| IGFBP2 | TGFbeta | IL8 | IL13 | 37.74% | 28.30% | 24.53% | 18.87% | 3.77% | 1.89% |
| IGFBP2 | TIMP1 | IL8 | EpCAM | 35.85% | 26.42% | 26.42% | 16.98% | 11.32% | 3.77% |
| IGFBP2 | Dkk3.S | IL8 | IL13 | 35.85% | 24.53% | 20.75% | 16.98% | 7.55% | 1.89% |
| IGFBP2 | M2PK | TGFbeta | IL13 | 35.85% | 20.75% | 20.75% | 16.98% | 16.98% | 7.55% |
| IGFBP2 | Mac2BP | TIMP1 | IL13 | 33.96% | 30.19% | 28.30% | 24.53% | 13.21% | 7.55% |
| IGFBP2 | Mac2BP | TGFbeta | EpCAM | 33.96% | 28.30% | 26.42% | 28.30% | 20.75% | 13.21% |
| IGFBP2 | M2PK | Mac2BP | TIMP1 | 33.96% | 28.30% | 28.30% | 24.53% | 18.87% | 11.32% |
| IGFBP2 | Dkk3.S | Mac2BP | TIMP1 | 33.96% | 28.30% | 28.30% | 22.64% | 18.87% | 9.43% |
| IGFBP2 | M2PK | Mac2BP | EpCAM | 33.96% | 28.30% | 24.53% | 20.75% | 16.98% | 11.32% |
| IGFBP2 | Mac2BP | IL8 | IL13 | 33.96% | 24.53% | 26.42% | 16.98% | 13.21% | 5.66% |
| IGFBP2 | IL8 | IL13 | EpCAM | 33.96% | 20.75% | 26.42% | 15.09% | 5.66% | 5.66% |
| IGFBP2 | Dkk3.S | Mac2BP | TGFbeta | 32.08% | 32.08% | 28.30% | 26.42% | 20.75% | 11.32% |
| IGFBP2 | Mac2BP | TGFbeta | IL13 | 32.08% | 30.19% | 30.19% | 20.75% | 18.87% | 11.32% |
| IGFBP2 | Dkk3 | TGFbeta | EpCAM | 32.08% | 30.19% | 26.42% | 18.87% | 18.87% | 9.43% |
| IGFBP2 | M2PK | Mac2BP | TGFbeta | 32.08% | 26.42% | 28.30% | 26.42% | 24.53% | 15.09% |
| IGFBP2 | TGFbeta | IL8 | EpCAM | 32.08% | 26.42% | 22.64% | 24.53% | 13.21% | 5.66% |
| IGFBP2 | Dkk3.S | Mac2BP | EpCAM | 32.08% | 26.42% | 22.64% | 22.64% | 16.98% | 11.32% |
| IGFBP2 | Mac2BP | IL13 | EpCAM | 32.08% | 26.42% | 32.08% | 18.87% | 13.21% | 9.43% |
| IGFBP2 | Dkk3.S | TGFbeta | TIMP1 | 32.08% | 26.42% | 22.64% | 15.09% | 15.09% | 7.55% |
| IGFBP2 | M2PK | TIMP1 | IL13 | 32.08% | 24.53% | 26.42% | 24.53% | 16.98% | 5.66% |
| IGFBP2 | Mac2BP | TIMP1 | IL8 | 32.08% | 24.53% | 30.19% | 20.75% | 15.09% | 9.43% |
| IGFBP2 | M2PK | TGFbeta | TIMP1 | 32.08% | 24.53% | 32.08% | 16.98% | 15.09% | 9.43% |
| IGFBP2 | Dkk3.S | M2PK | IL13 | 32.08% | 24.53% | 28.30% | 13.21% | 11.32% | 7.55% |
| IGFBP2 | TIMP1 | IL13 | EpCAM | 32.08% | 22.64% | 28.30% | 22.64% | 11.32% | 1.89% |
| IGFBP2 | M2PK | TIMP1 | EpCAM | 32.08% | 22.64% | 28.30% | 20.75% | 13.21% | 13.21% |
| IGFBP2 | Dkk3.S | M2PK | TGFbeta | 32.08% | 22.64% | 28.30% | 20.75% | 20.75% | 7.55% |
| IGFBP2 | Dkk3.S | TIMP1 | EpCAM | 32.08% | 22.64% | 28.30% | 16.98% | 11.32% | 11.32% |
| IGFBP2 | Dkk3.S | M2PK | EpCAM | 32.08% | 22.64% | 26.42% | 15.09% | 9.43% | 7.55% |
| IGFBP2 | Dkk3.S | TIMP1 | IL8 | 32.08% | 20.75% | 24.53% | 18.87% | 9.43% | 3.77% |
| IGFBP2 | M2PK | IL13 | EpCAM | 32.08% | 20.75% | 20.75% | 13.21% | 11.32% | 11.32% |
| IGFBP2 | Dkk3.S | M2PK | TIMP1 | 32.08% | 18.87% | 28.30% | 16.98% | 16.98% | 13.21% |
| Mac2BP | TIMP1 | IL8 | IL13 | 32.08% | 18.87% | 20.75% | 13.21% | 11.32% | 7.55% |
| Dkk3.S | TIMP1 | IL8 | IL13 | 32.08% | 16.98% | 16.98% | 15.09% | 11.32% | 7.55% |
| IGFBP2 | Dkk3.S | M2PK | Mac2BP | 30.19% | 28.30% | 30.19% | 20.75% | 15.09% | 13.21% |
| IGFBP2 | TGFbeta | TIMP1 | EpCAM | 30.19% | 26.42% | 28.30% | 22.64% | 7.55% | 3.77% |
| IGFBP2 | Dkk3.S | TGFbeta | IL13 | 30.19% | 26.42% | 24.53% | 20.75% | 16.98% | 5.66% |
| IGFBP2 | Mac2BP | IL8 | EpCAM | 30.19% | 24.53% | 28.30% | 22.64% | 20.75% | 7.55% |
| IGFBP2 | Dkk3.S | TIMP1 | IL13 | 30.19% | 24.53% | 28.30% | 22.64% | 15.09% | 1.89% |
| IGFBP2 | M2PK | TGFbeta | EpCAM | 30.19% | 24.53% | 28.30% | 20.75% | 20.75% | 7.55% |
| IGFBP2 | M2PK | TGFbeta | IL8 | 30.19% | 24.53% | 22.64% | 20.75% | 9.43% | 9.43% |
| IGFBP2 | M2PK | Mac2BP | IL13 | 30.19% | 24.53% | 22.64% | 18.87% | 15.09% | 11.32% |
| IGFBP2 | TGFbeta | TIMP1 | IL8 | 30.19% | 24.53% | 22.64% | 18.87% | 11.32% | 7.55% |
| IGFBP2 | TGFbeta | IL13 | EpCAM | 30.19% | 24.53% | 28.30% | 15.09% | 9.43% | 5.66% |
| IGFBP2 | Mac2BP | TGFbeta | IL8 | 30.19% | 22.64% | 26.42% | 20.75% | 18.87% | 15.09% |
| IGFBP2 | M2PK | IL8 | IL13 | 30.19% | 22.64% | 26.42% | 16.98% | 5.66% | 3.77% |
| IGFBP2 | Dkk3.S | Mac2BP | IL13 | 30.19% | 22.64% | 28.30% | 16.98% | 16.98% | 9.43% |

TABLE 15(b)
Four biomarker, ten-fold cross validated combinations plus age having >30% sensitivity
at 86.4% specificity.

| | | | | Sensitivity | Sensitivity | Sensitivity |
|---|---|---|---|---|---|---|
| | | | | at 86.4% | at 90% | at 95% |
| | | | | specificity | specificity | specificity |
| Biomarkers | | | | cross-validated | (cross-validated) | (cross-validated) |
| IGFBP2 | Mac2BP | TGFbeta | DKK3 | 32.08% | 26.4% | 11.3% |
| IGFBP2 | Mac2BP | TGFbeta | TIMP1 | 32.08% | 28.3% | 9.4% |
| IGFBP2 | Mac2BP | EpCAM | TIMP1 | 32.08% | 24.5% | 9.4% |
| IGFBP2 | Mac2BP | IL-13 | TIMP1 | 30.19% | 24.5% | 7.5% |
| IGFBP2 | Mac2BP | TGFbeta | IL-13 | 30.18% | 20.7% | 11.3% |
| IGFBP2 | EpCAM | TGFbeta | DKK3 | 30.19% | 18.9% | 9.4% |

TABLE 16(a)
Five biomarker, non-cross validated combinations plus age having >30% sensitivity at 86.4% specificity.
Combinations also showing a cross validated sensitivity >30% at 86.4% specificity are indicated in bold face.
Non-XV - Sensitivity value not cross validated, XV - Cross validated sensitivity value.

| | | | | | Sensitivity | | Sensitivity | | Sensitivity | |
|---|---|---|---|---|---|---|---|---|---|---|
| | | | | | at 86.4% | | at 90% | | at 95% | |
| | | | | | Specificity | | Specificity | | Specificity | |
| Biomarker | | | | | Non XV | XV | Non XV | XV | Non XV | XV |
| IGFBP2 | Dkk3 | Mac2BP | TIMP1 | EpCAM | 39.62% | 28.30% | 30.19% | 15.09% | 20.75% | 7.55% |
| IGFBP2 | TGFbeta | TIMP1 | IL8 | IL13 | 39.62% | 28.30% | 28.30% | 13.21% | 5.66% | 1.89% |
| IGFBP2 | Mac2BP | TGFbeta | IL8 | IL13 | 39.62% | 26.42% | 26.42% | 18.87% | 13.21% | 7.55% |
| IGFBP2 | Dkk3 | TIMP1 | IL8 | IL13 | 39.62% | 26.42% | 26.42% | 13.21% | 3.77% | 1.89% |
| IGFBP2 | Dkk3 | Mac2BP | IL8 | IL13 | 39.62% | 20.75% | 22.64% | 16.98% | 11.32% | 5.66% |
| IGFBP2 | Mac2BP | TGFbeta | TIMP1 | EpCAM | 37.74% | 32.08% | 33.96% | 26.42% | 26.42% | 13.21% |
| IGFBP2 | M2PK | Mac2BP | TGFbeta | TIMP1 | 37.74% | 30.19% | 28.30% | 22.64% | 20.75% | 11.32% |
| IGFBP2 | TIMP1 | IL8 | IL13 | EpCAM | 37.74% | 28.30% | 26.42% | 20.75% | 3.77% | 1.89% |
| IGFBP2 | Mac2BP | TIMP1 | IL8 | IL13 | 37.74% | 26.42% | 30.19% | 20.75% | 15.09% | 3.77% |
| IGFBP2 | Dkk3 | TGFbeta | IL8 | IL13 | 37.74% | 24.53% | 28.30% | 16.98% | 7.55% | 3.77% |
| IGFBP2 | Mac2BP | TIMP1 | IL13 | EpCAM | 35.85% | 28.30% | 35.85% | 22.64% | 16.98% | 3.77% |
| IGFBP2 | Dkk3 | M2PK | Mac2BP | TGFbeta | 35.85% | 28.30% | 28.30% | 22.64% | 24.53% | 15.09% |
| IGFBP2 | TGFbeta | IL8 | IL13 | EpCAM | 35.85% | 28.30% | 22.64% | 16.98% | 7.55% | 1.89% |
| IGFBP2 | Mac2BP | TIMP1 | IL8 | EpCAM | 35.85% | 26.42% | 30.19% | 22.64% | 16.98% | 13.21% |
| IGFBP2 | M2PK | Mac2BP | TGFbeta | EpCAM | 35.85% | 26.42% | 24.53% | 18.87% | 22.64% | 15.09% |
| IGFBP2 | TGFbeta | TIMP1 | IL8 | EpCAM | 35.85% | 26.42% | 26.42% | 16.98% | 7.55% | 9.43% |
| IGFBP2 | Dkk3 | IL8 | IL13 | EpCAM | 35.85% | 20.75% | 20.75% | 16.98% | 7.55% | 3.77% |
| IGFBP2 | Mac2BP | TGFbeta | TIMP1 | IL13 | 33.96% | 30.19% | 28.30% | 24.53% | 16.98% | 7.55% |
| IGFBP2 | M2PK | TIMP1 | IL8 | IL13 | 33.96% | 28.30% | 26.42% | 20.75% | 9.43% | 1.89% |
| IGFBP2 | Dkk3 | Mac2BP | TIMP1 | IL13 | 33.96% | 28.30% | 30.19% | 18.87% | 15.09% | 7.55% |
| IGFBP2 | Mac2BP | IL8 | IL13 | EpCAM | 33.96% | 28.30% | 28.30% | 15.09% | 11.32% | 5.66% |
| IGFBP2 | Dkk3 | M2PK | TGFbeta | IL13 | 33.96% | 26.42% | 20.75% | 16.98% | 15.09% | 7.55% |
| IGFBP2 | M2PK | TIMP1 | IL13 | EpCAM | 33.96% | 24.53% | 28.30% | 24.53% | 18.87% | 3.77% |
| IGFBP2 | Dkk3 | M2PK | Mac2BP | TIMP1 | 33.96% | 24.53% | 26.42% | 22.64% | 18.87% | 11.32% |
| IGFBP2 | Dkk3 | TGFbeta | TIMP1 | EpCAM | 33.96% | 24.53% | 28.30% | 18.87% | 7.55% | 5.66% |
| IGFBP2 | M2PK | Mac2BP | IL8 | IL13 | 33.96% | 24.53% | 28.30% | 18.87% | 13.21% | 7.55% |
| IGFBP2 | M2PK | TIMP1 | IL8 | EpCAM | 33.96% | 18.87% | 24.53% | 18.87% | 13.21% | 7.55% |
| IGFBP2 | M2PK | TGFbeta | IL13 | EpCAM | 33.96% | 18.87% | 28.30% | 16.98% | 16.98% | 5.66% |
| IGFBP2 | Dkk3 | Mac2BP | TGFbeta | TIMP1 | 32.08% | 30.19% | 28.30% | 26.42% | 20.75% | 9.43% |
| IGFBP2 | Dkk3 | Mac2BP | TGFbeta | EpCAM | 32.08% | 28.30% | 28.30% | 24.53% | 18.87% | 15.09% |
| IGFBP2 | M2PK | Mac2BP | TIMP1 | IL13 | 32.08% | 26.42% | 32.08% | 24.53% | 18.87% | 9.43% |
| IGFBP2 | TGFbeta | TIMP1 | IL13 | EpCAM | 32.08% | 26.42% | 28.30% | 20.75% | 11.32% | 3.77% |
| IGFBP2 | Mac2BP | TGFbeta | TIMP1 | IL8 | 32.08% | 26.42% | 30.19% | 20.75% | 15.09% | 11.32% |
| IGFBP2 | Mac2BP | TGFbeta | IL13 | EpCAM | 32.08% | 26.42% | 30.19% | 18.87% | 13.21% | 7.55% |
| IGFBP2 | M2PK | TGFbeta | IL8 | EpCAM | 32.08% | 26.42% | 26.42% | 18.87% | 15.09% | 3.77% |
| IGFBP2 | M2PK | Mac2BP | IL8 | EpCAM | 32.08% | 24.53% | 26.42% | 24.53% | 16.98% | 11.32% |
| IGFBP2 | M2PK | TGFbeta | TIMP1 | IL13 | 32.08% | 24.53% | 26.42% | 22.64% | 13.21% | 7.55% |
| IGFBP2 | Dkk3 | M2PK | Mac2BP | EpCAM | 32.08% | 24.53% | 26.42% | 20.75% | 16.98% | 11.32% |
| IGFBP2 | M2PK | TGFbeta | TIMP1 | EpCAM | 32.08% | 22.64% | 28.30% | 22.64% | 18.87% | 3.77% |
| IGFBP2 | M2PK | Mac2BP | TIMP1 | IL8 | 32.08% | 22.64% | 24.53% | 22.64% | 20.75% | 11.32% |
| IGFBP2 | M2PK | Mac2BP | TGFbeta | IL13 | 32.08% | 22.64% | 24.53% | 18.87% | 16.98% | 11.32% |
| IGFBP2 | M2PK | IL8 | IL13 | EpCAM | 32.08% | 22.64% | 24.53% | 16.98% | 5.66% | 3.77% |
| IGFBP2 | M2PK | Mac2BP | IL13 | EpCAM | 32.08% | 22.64% | 26.42% | 16.98% | 15.09% | 11.32% |
| IGFBP2 | Dkk3 | TGFbeta | TIMP1 | IL8 | 32.08% | 22.64% | 24.53% | 16.98% | 11.32% | 3.77% |
| IGFBP2 | Dkk3 | M2PK | TGFbeta | TIMP1 | 32.08% | 22.64% | 32.08% | 15.09% | 16.98% | 9.43% |
| IGFBP2 | Dkk3 | M2PK | IL8 | IL13 | 32.08% | 20.75% | 22.64% | 16.98% | 5.66% | 3.77% |
| IGFBP2 | Dkk3 | M2PK | TIMP1 | IL8 | 32.08% | 20.75% | 24.53% | 16.98% | 13.21% | 7.55% |
| IGFBP2 | Dkk3 | Mac2BP | TIMP1 | IL8 | 32.08% | 18.87% | 30.19% | 18.87% | 18.87% | 16.98% |
| IGFBP2 | Dkk3 | M2PK | TIMP1 | EpCAM | 32.08% | 18.87% | 28.30% | 16.98% | 18.87% | 15.09% |
| IGFBP2 | Dkk3 | M2PK | IL13 | EpCAM | 32.08% | 18.87% | 28.30% | 13.21% | 11.32% | 7.55% |
| Dkk3 | TGFbeta | TIMP1 | IL8 | IL13 | 32.08% | 16.98% | 16.98% | 11.32% | 11.32% | 7.55% |
| Dkk3 | Mac2BP | TIMP1 | IL8 | IL13 | 32.08% | 16.98% | 18.87% | 11.32% | 11.32% | 5.66% |
| IGFBP2 | Dkk3 | Mac2BP | TGFbeta | IL13 | 30.19% | 30.19% | 28.30% | 16.98% | 18.87% | 11.32% |
| IGFBP2 | Dkk3 | TGFbeta | IL13 | EpCAM | 30.19% | 28.30% | 28.30% | 20.75% | 15.09% | 5.66% |
| IGFBP2 | Dkk3 | M2PK | TIMP1 | IL13 | 30.19% | 26.42% | 26.42% | 20.75% | 20.75% | 7.55% |
| IGFBP2 | M2PK | TGFbeta | IL8 | IL13 | 30.19% | 24.53% | 26.42% | 20.75% | 9.43% | 3.77% |
| IGFBP2 | Dkk3 | M2PK | TGFbeta | EpCAM | 30.19% | 24.53% | 30.19% | 16.98% | 20.75% | 11.32% |
| IGFBP2 | Dkk3 | TIMP1 | IL8 | EpCAM | 30.19% | 24.53% | 30.19% | 15.09% | 15.09% | 3.77% |
| IGFBP2 | Dkk3 | TIMP1 | IL13 | EpCAM | 30.19% | 22.64% | 28.30% | 22.64% | 13.21% | 3.77% |
| IGFBP2 | Dkk3 | Mac2BP | IL13 | EpCAM | 30.19% | 22.64% | 30.19% | 18.87% | 15.09% | 9.43% |
| IGFBP2 | Dkk3 | M2PK | Mac2BP | IL13 | 30.19% | 22.64% | 28.30% | 18.87% | 15.09% | 9.43% |
| Dkk3 | M2PK | Mac2BP | TGFbeta | IL8 | 30.19% | 9.43% | 16.98% | 5.66% | 9.43% | 3.77% |

TABLE 16(b)
Five biomarker, ten-fold cross validated combinations plus age having >30%
sensitivity at 86.4% specificity.

| | | | | | Sensitivity | Sensitivity | Sensitivity |
|---|---|---|---|---|---|---|---|
| | | | | | at 86.4% | at 90% | at 95% |
| | | | | | specificity | specificity | specificity |
| Biomarkers | | | | | cross-validated | (cross-validated) | (cross-validated) |
| IGFBP2 | Mac2BP | TGFbeta | TIMP1 | EpCAM | 32.08 | 26.4 | 13.2 |
| IGFBP2 | Mac2BP | TGFbeta | TIMP1 | M2PK | 30.19 | 22.6 | 11.3 |
| IGFBP2 | Mac2BP | TGFbeta | DKK3 | IL-13 | 30.19 | 17.0 | 11.3 |
| IGFBP2 | Mac2BP | TGFbeta | TIMP1 | IL-13 | 30.19 | 24.3 | 7.5 |
| IGFBP2 | Mac2BP | TGFbeta | TIMP1 | DKK3 | 30.19 | 26.4 | 9.4 |

TABLE 17(a)
Six biomarker, non-cross validated combinations plus age having >30% sensitivity at 86.4% specificity.
Combinations also showing a cross validated sensitivity >30% at 86.4% specificity are indicated in bold face.
Non-XV - Sensitivity value not cross validated, XV - Cross validated sensitivity value.

| | | | | | | Sensitivity | | Sensitivity | | Sensitivity | |
|---|---|---|---|---|---|---|---|---|---|---|---|
| | | | | | | at 86.4% | | at 90% | | at 95% | |
| | | | | | | Specificity | | Specificity | | Specificity | |
| Biomarker | | | | | | Non XV | XV | Non XV | XV | Non XV | XV |
| IGFBP2 | M2PK | Mac2BP | TGFbeta | TIMP1 | EpCAM | 39.62% | 28.30% | 32.08% | 26.42% | 18.87% | 9.43% |
| IGFBP2 | Dkk3 | TIMP1 | IL8 | IL13 | EpCAM | 39.62% | 24.53% | 26.42% | 18.87% | 3.77% | 1.89% |
| IGFBP2 | Dkk3 | M2PK | Mac2BP | TIMP1 | EpCAM | 39.62% | 22.64% | 28.30% | 18.87% | 16.98% | 7.55% |
| IGFBP2 | Dkk3 | Mac2BP | TGFbeta | IL8 | IL13 | 39.62% | 22.64% | 26.42% | 15.09% | 16.98% | 5.66% |
| IGFBP2 | Dkk3 | Mac2BP | IL8 | IL13 | EpCAM | 39.62% | 18.87% | 24.53% | 16.98% | 13.21% | 5.66% |
| IGFBP2 | Dkk3 | Mac2BP | TGFbeta | TIMP1 | EpCAM | 37.74% | 28.30% | 32.08% | 22.64% | 24.53% | 5.66% |
| IGFBP2 | TGFbeta | TIMP1 | IL8 | IL13 | EpCAM | 37.74% | 28.30% | 28.30% | 16.98% | 5.66% | 1.89% |
| IGFBP2 | Mac2BP | TGFbeta | TIMP1 | IL8 | IL13 | 37.74% | 26.42% | 30.19% | 20.75% | 16.98% | 3.77% |
| IGFBP2 | Dkk3 | TGFbeta | TIMP1 | IL8 | IL13 | 37.74% | 26.42% | 26.42% | 15.09% | 5.66% | 1.89% |
| IGFBP2 | Dkk3 | Mac2BP | TIMP1 | IL8 | IL13 | 37.74% | 24.53% | 30.19% | 15.09% | 16.98% | 3.77% |
| IGFBP2 | Mac2BP | TGFbeta | TIMP1 | IL13 | EpCAM | 35.85% | 28.30% | 33.96% | 22.64% | 13.21% | 3.77% |
| IGFBP2 | Dkk3 | TGFbeta | IL8 | IL13 | EpCAM | 35.85% | 28.30% | 28.30% | 16.98% | 7.55% | 1.89% |
| IGFBP2 | Mac2BP | TGFbeta | IL8 | IL13 | EpCAM | 35.85% | 28.30% | 24.53% | 13.21% | 15.09% | 5.66% |
| IGFBP2 | Mac2BP | TIMP1 | IL8 | IL13 | EpCAM | 35.85% | 26.42% | 32.08% | 18.87% | 9.43% | 3.77% |
| IGFBP2 | M2PK | Mac2BP | TGFbeta | IL8 | IL13 | 35.85% | 26.42% | 28.30% | 18.87% | 20.75% | 5.66% |
| IGFBP2 | M2PK | TGFbeta | IL8 | IL13 | EpCAM | 35.85% | 24.53% | 26.42% | 20.75% | 9.43% | 3.77% |
| IGFBP2 | Dkk3 | Mac2BP | TIMP1 | IL13 | EpCAM | 35.85% | 22.64% | 32.08% | 18.87% | 16.98% | 5.66% |
| IGFBP2 | Dkk3 | M2PK | Mac2BP | TGFbeta | EpCAM | 35.85% | 20.75% | 24.53% | 20.75% | 24.53% | 13.21% |
| IGFBP2 | M2PK | Mac2BP | TIMP1 | IL8 | EpCAM | 35.85% | 20.75% | 30.19% | 18.87% | 15.09% | 9.43% |
| IGFBP2 | M2PK | Mac2BP | TIMP1 | IL13 | EpCAM | 33.96% | 28.30% | 32.08% | 22.64% | 15.09% | 7.55% |
| IGFBP2 | M2PK | Mac2BP | TIMP1 | IL8 | IL13 | 33.96% | 26.42% | 28.30% | 20.75% | 22.64% | 9.43% |
| IGFBP2 | M2PK | TGFbeta | TIMP1 | IL13 | EpCAM | 33.96% | 24.53% | 28.30% | 24.53% | 13.21% | 3.77% |
| IGFBP2 | M2PK | Mac2BP | TGFbeta | IL8 | EpCAM | 33.96% | 24.53% | 30.19% | 20.75% | 18.87% | 11.32% |
| IGFBP2 | Dkk3 | M2PK | Mac2BP | TIMP1 | IL13 | 33.96% | 24.53% | 30.19% | 20.75% | 18.87% | 9.43% |
| IGFBP2 | Dkk3 | M2PK | Mac2BP | TGFbeta | IL13 | 33.96% | 24.53% | 26.42% | 18.87% | 15.09% | 11.32% |
| IGFBP2 | Dkk3 | M2PK | TGFbeta | IL13 | EpCAM | 33.96% | 24.53% | 24.53% | 16.98% | 15.09% | 5.66% |
| IGFBP2 | M2PK | TGFbeta | TIMP1 | IL8 | EpCAM | 33.96% | 22.64% | 24.53% | 18.87% | 20.75% | 7.55% |
| IGFBP2 | M2PK | Mac2BP | IL8 | IL13 | EpCAM | 33.96% | 22.64% | 28.30% | 16.98% | 11.32% | 5.66% |
| IGFBP2 | Dkk3 | M2PK | TIMP1 | IL8 | EpCAM | 33.96% | 18.87% | 22.64% | 16.98% | 15.09% | 9.43% |
| IGFBP2 | Dkk3 | Mac2BP | TGFbeta | TIMP1 | IL13 | 32.08% | 30.19% | 30.19% | 16.98% | 15.09% | 7.55% |
| IGFBP2 | M2PK | TIMP1 | IL8 | IL13 | EpCAM | 32.08% | 26.42% | 28.30% | 20.75% | 9.43% | 1.89% |
| IGFBP2 | Dkk3 | M2PK | TIMP1 | IL13 | EpCAM | 32.08% | 26.42% | 28.30% | 18.87% | 20.75% | 5.66% |
| IGFBP2 | Dkk3 | Mac2BP | TGFbeta | IL13 | EpCAM | 32.08% | 26.42% | 30.19% | 18.87% | 15.09% | 7.55% |
| IGFBP2 | Dkk3 | M2PK | TGFbeta | TIMP1 | IL13 | 32.08% | 26.42% | 26.42% | 18.87% | 13.21% | 7.55% |
| IGFBP2 | Dkk3 | M2PK | Mac2BP | TGFbeta | TIMP1 | 32.08% | 26.42% | 28.30% | 18.87% | 20.75% | 11.32% |
| IGFBP2 | M2PK | Mac2BP | TGFbeta | TIMP1 | IL13 | 32.08% | 24.53% | 28.30% | 22.64% | 16.98% | 11.32% |
| IGFBP2 | Dkk3 | M2PK | TIMP1 | IL8 | IL13 | 32.08% | 24.53% | 26.42% | 18.87% | 11.32% | 1.89% |
| IGFBP2 | Dkk3 | M2PK | Mac2BP | TGFbeta | IL8 | 32.08% | 22.64% | 24.53% | 20.75% | 16.98% | 15.09% |
| IGFBP2 | Dkk3 | Mac2BP | TGFbeta | TIMP1 | IL8 | 32.08% | 22.64% | 26.42% | 18.87% | 20.75% | 16.98% |
| IGFBP2 | Dkk3 | M2PK | Mac2BP | IL8 | IL13 | 32.08% | 22.64% | 24.53% | 16.98% | 15.09% | 7.55% |
| IGFBP2 | Dkk3 | M2PK | Mac2BP | IL13 | EpCAM | 32.08% | 20.75% | 30.19% | 15.09% | 15.09% | 9.43% |
| IGFBP2 | Dkk3 | M2PK | TGFbeta | TIMP1 | EpCAM | 32.08% | 20.75% | 28.30% | 15.09% | 24.53% | 5.66% |
| IGFBP2 | Dkk3 | M2PK | IL8 | IL13 | EpCAM | 32.08% | 16.98% | 20.75% | 15.09% | 5.66% | 3.77% |
| IGFBP2 | Mac2BP | TGFbeta | TIMP1 | IL8 | EpCAM | 30.19% | 30.19% | 30.19% | 26.42% | 20.75% | 13.21% |
| IGFBP2 | M2PK | TGFbeta | TIMP1 | IL8 | IL13 | 30.19% | 26.42% | 28.30% | 16.98% | 11.32% | 1.89% |
| IGFBP2 | Dkk3 | M2PK | TGFbeta | IL8 | IL13 | 30.19% | 24.53% | 26.42% | 20.75% | 9.43% | 1.89% |
| IGFBP2 | Dkk3 | Mac2BP | TIMP1 | IL8 | EpCAM | 30.19% | 24.53% | 30.19% | 15.09% | 20.75% | 11.32% |
| IGFBP2 | Dkk3 | M2PK | TGFbeta | IL8 | EpCAM | 30.19% | 24.53% | 28.30% | 15.09% | 15.09% | 9.43% |
| IGFBP2 | Dkk3 | TGFbeta | TIMP1 | IL13 | EpCAM | 30.19% | 22.64% | 28.30% | 20.75% | 11.32% | 5.66% |
| IGFBP2 | Dkk3 | Mac2BP | TGFbeta | IL8 | EpCAM | 30.19% | 22.64% | 26.42% | 20.75% | 22.64% | 13.21% |
| IGFBP2 | Dkk3 | M2PK | Mac2BP | IL8 | EpCAM | 30.19% | 22.64% | 26.42% | 18.87% | 15.09% | 13.21% |
| IGFBP2 | Dkk3 | TGFbeta | TIMP1 | IL8 | EpCAM | 30.19% | 22.64% | 26.42% | 15.09% | 11.32% | 3.77% |
| IGFBP2 | M2PK | Mac2BP | TGFbeta | IL13 | EpCAM | 30.19% | 20.75% | 28.30% | 16.98% | 16.98% | 11.32% |
| IGFBP2 | Dkk3 | M2PK | TGFbeta | TIMP1 | IL8 | 30.19% | 20.75% | 28.30% | 15.09% | 13.21% | 9.43% |
| Dkk3 | TGFbeta | TIMP1 | IL8 | IL13 | EpCAM | 30.19% | 15.09% | 20.75% | 13.21% | 11.32% | 1.89% |
| Dkk3 | Mac2BP | TIMP1 | IL8 | IL13 | EpCAM | 30.19% | 15.09% | 18.87% | 7.55% | 11.32% | 5.66% |

TABLE 17(b)
Six biomarker, ten-fold cross validated combinations plus age having >30% sensitivity at
86.4% specificity.

| | | | | | | Sensitivity | Sensitivity | Sensitivity |
|---|---|---|---|---|---|---|---|---|
| | | | | | | at 86.4% | at 90% | at 95% |
| | | | | | | specificity | specificity | specificity |
| Biomarkers | | | | | | cross-validated | (cross-validated) | (cross-validated) |
| IGFBP2 | Mac2BP | TGFbeta | TIMP1 | IL-8 | EpCAM | 30.19 | 26.4 | 13.2 |
| IGFBP2 | Mac2BP | TGFbeta | TIMP1 | DKK3 | IL-13 | 30.19 | 17.0 | 7.5 |

TABLE 18
Seven-biomarker, non-cross validated combinations plus age having >30% sensitivity at 86.4% specificity.
No seven biomarker panels showed cross validated sensitivity >30% at 86.4% specificity.
Non-XV - Sensitivity value not cross validated, XV - Cross validated sensitivity value.

| | | | | | | | Sensitivity | | Sensitivity | | Sensitivity | |
|---|---|---|---|---|---|---|---|---|---|---|---|---|
| | | | | | | | at 86.4% | | at 90% | | at 95% | |
| | | | | | | | specificity | | specificity | | specificity | |
| Biomarker | | | | | | | Non XV | XV | Non XV | XV | Non XV | XV |
| IGFBP2 | M2PK | Mac2BP | TGFbeta | TIMP1 | IL8 | EPCAM | 37.74% | 22.64% | 32.08% | 22.64% | 20.75% | 9.43% |
| IGFBP2 | Dkk3 | M2PK | Mac2BP | TGFbeta | TIMP1 | EPCAM | 37.74% | 22.64% | 26.42% | 20.75% | 20.75% | 5.66% |
| IGFBP2 | Dkk3 | Mac2BP | TGFbeta | TIMP1 | IL8 | IL13 | 37.74% | 22.64% | 30.19% | 16.98% | 16.98% | 3.77% |
| IGFBP2 | M2PK | Mac2BP | TGFbeta | IL8 | IL13 | EPCAM | 37.74% | 22.64% | 28.30% | 15.09% | 18.87% | 3.77% |
| IGFBP2 | M2PK | Mac2BP | TGFbeta | TIMP1 | IL13 | EPCAM | 35.85% | 26.42% | 32.08% | 22.64% | 15.09% | 11.32% |
| IGFBP2 | M2PK | Mac2BP | TGFbeta | TIMP1 | IL8 | IL13 | 35.85% | 26.42% | 30.19% | 20.75% | 18.87% | 9.43% |
| IGFBP2 | Mac2BP | TGFbeta | TIMP1 | IL8 | IL13 | EPCAM | 35.85% | 26.42% | 33.96% | 18.87% | 9.43% | 3.77% |
| IGFBP2 | Dkk3 | M2PK | Mac2BP | TGFbeta | IL8 | IL13 | 35.85% | 26.42% | 26.42% | 18.87% | 22.64% | 5.66% |
| IGFBP2 | Dkk3 | Mac2BP | TIMP1 | IL8 | IL13 | EPCAM | 35.85% | 26.42% | 32.08% | 15.09% | 15.09% | 1.89% |
| IGFBP2 | Dkk3 | M2PK | TGFbeta | TIMP1 | IL13 | EPCAM | 35.85% | 24.53% | 28.30% | 20.75% | 15.09% | 5.66% |
| IGFBP2 | Dkk3 | M2PK | Mac2BP | TIMP1 | IL13 | EPCAM | 35.85% | 22.64% | 32.08% | 18.87% | 18.87% | 7.55% |
| IGFBP2 | Dkk3 | TGFbeta | TIMP1 | IL8 | IL13 | EPCAM | 35.85% | 22.64% | 26.42% | 15.09% | 3.77% | 1.89% |
| IGFBP2 | Dkk3 | Mac2BP | TGFbeta | IL8 | IL13 | EPCAM | 35.85% | 22.64% | 30.19% | 11.32% | 18.87% | 3.77% |
| IGFBP2 | Dkk3 | M2PK | Mac2BP | TIMP1 | IL8 | EPCAM | 35.85% | 16.98% | 30.19% | 15.09% | 18.87% | 13.21% |
| IGFBP2 | Dkk3 | M2PK | Mac2BP | TGFbeta | TIMP1 | IL13 | 33.96% | 26.42% | 28.30% | 18.87% | 16.98% | 9.43% |
| IGFBP2 | M2PK | TGFbeta | TIMP1 | IL8 | IL13 | EPCAM | 33.96% | 24.53% | 30.19% | 18.87% | 9.43% | 1.89% |
| IGFBP2 | M2PK | Mac2BP | TIMP1 | IL8 | IL13 | EPCAM | 33.96% | 24.53% | 32.08% | 18.87% | 20.75% | 3.77% |
| IGFBP2 | Dkk3 | Mac2BP | TGFbeta | TIMP1 | IL13 | EPCAM | 33.96% | 24.53% | 33.96% | 16.98% | 15.09% | 5.66% |
| IGFBP2 | Dkk3 | M2PK | TIMP1 | IL8 | IL13 | EPCAM | 33.96% | 24.53% | 26.42% | 15.09% | 11.32% | 1.89% |
| IGFBP2 | Dkk3 | M2PK | Mac2BP | TIMP1 | IL8 | IL13 | 33.96% | 22.64% | 28.30% | 16.98% | 22.64% | 7.55% |
| IGFBP2 | Dkk3 | M2PK | TGFbeta | TIMP1 | IL8 | EPCAM | 33.96% | 22.64% | 26.42% | 15.09% | 18.87% | 5.66% |
| IGFBP2 | Dkk3 | M2PK | TGFbeta | TIMP1 | IL8 | IL13 | 32.08% | 22.64% | 26.42% | 18.87% | 13.21% | 1.89% |
| IGFBP2 | Dkk3 | M2PK | Mac2BP | TGFbeta | IL13 | EPCAM | 32.08% | 18.87% | 26.42% | 18.87% | 16.98% | 11.32% |
| IGFBP2 | Dkk3 | M2PK | Mac2BP | IL8 | IL13 | EPCAM | 32.08% | 16.98% | 24.53% | 15.09% | 15.09% | 7.55% |
| IGFBP2 | Dkk3 | Mac2BP | TGFbeta | TIMP1 | IL8 | EPCAM | 30.19% | 24.53% | 30.19% | 16.98% | 22.64% | 13.21% |
| IGFBP2 | Dkk3 | M2PK | TGFbeta | IL8 | IL13 | EPCAM | 30.19% | 20.75% | 28.30% | 20.75% | 9.43% | 1.89% |

TABLE 19
Eight-biomarker, non-cross validated combinations plus age having >30% sensitivity at 86.4% specificity.
No eight-biomarker panels showed a cross validated sensitivity >30% at 86.4% specificity.
Non-XV - Sensitivity value not cross validated, XV - Cross validated sensitivity value.

| | | | | | | | | Sensitivity | | Sensitivity | | Sensitivity | |
|---|---|---|---|---|---|---|---|---|---|---|---|---|---|
| | | | | | | | | at 86.4% | | at 90% | | at 95% | |
| | | | | | | | | Specificity | | Specificity | | Specificity | |
| Biomarker | | | | | | | | Non XV | XV | Non XV | XV | Non XV | XV |
| IGFBP2 | Dkk3 | M2PK | Mac2BP | TGFbeta | TIMP1 | IL8 | EPCAM | 37.74% | 20.75% | 26.42% | 16.98% | 20.75% | 13.21% |
| IGFBP2 | Dkk3 | Mac2BP | TGFbeta | TIMP1 | IL8 | IL13 | EPCAM | 35.85% | 26.42% | 33.96% | 15.09% | 13.21% | 3.77% |
| IGFBP2 | M2PK | Mac2BP | TGFbeta | TIMP1 | IL8 | IL13 | EPCAM | 35.85% | 24.53% | 32.08% | 20.75% | 18.87% | 5.66% |
| IGFBP2 | Dkk3 | M2PK | TGFbeta | TIMP1 | IL8 | IL13 | EPCAM | 35.85% | 22.64% | 26.42% | 18.87% | 13.21% | 1.89% |
| IGFBP2 | Dkk3 | M2PK | Mac2BP | TGFbeta | IL8 | IL13 | EPCAM | 35.85% | 20.75% | 26.42% | 16.98% | 20.75% | 5.66% |
| IGFBP2 | Dkk3 | M2PK | Mac2BP | TIMP1 | IL8 | IL13 | EPCAM | 33.96% | 24.53% | 28.30% | 16.98% | 20.75% | 5.66% |
| IGFBP2 | Dkk3 | M2PK | Mac2BP | TGFbeta | TIMP1 | IL13 | EPCAM | 33.96% | 22.64% | 32.08% | 20.75% | 16.98% | 9.43% |
| IGFBP2 | Dkk3 | M2PK | Mac2BP | TGFbeta | TIMP1 | IL8 | IL13 | 33.96% | 20.75% | 30.19% | 16.98% | 20.75% | 5.66% |

TABLE 20
Nine-biomarker, non-cross validated combination plus age having >30% sensitivity at 86.4%
specificity. The nine-biomarker panel did not show cross validated sensitivity >30% at 86.4%
specificity. Non-XV—Sensitivity value not cross validated, XV—Cross validated sensitivity value.

| | | | | | | | | | Sensitivity at | | Sensitivity at | | Sensitivity at | |
|---|---|---|---|---|---|---|---|---|---|---|---|---|---|---|
| | | | | | | | | | 86.4% Specificity | | 90% Specificity | | 95% Specificity | |
| Biomarker | | | | | | | | | Non XV | XV | Non XV | XV | Non XV | XV |
| IGFBP2 | Dkk3 | M2PK | Mac2BP | TGFbeta | TIMP1 | IL8 | IL13 | EpCAM | 33.96% | 18.87% | 32.08% | 16.98% | 20.75% | 3.77% |

No seven, eight and nine biomarker panels plus age produced 10-fold cross validated models that differentiated between APA and Negative with a sensitivity >30% at 86.4% specificity.

Results in Tables 21 to 28 show the impact of including gender as a demographic term in the algorithm on the number, nature and performance of biomarker combinations (plus gender) detecting APA with a sensitivity of greater than 30% at 86.4% specificity. In tables labelled (a), biomarker combinations (plus gender) are ranked from top to bottom based on their non-cross validated Sensitivity value determined at 86.4% Specificity. Corresponding cross validated Sensitivity values for these top performing combinations are also shown. Combinations for which the cross validated sensitivity also exceeds 30% at 86.4% specificity are indicated in boldface. Tables labelled (b) show data only for those biomarker combinations (plus gender) producing ten-fold cross validated sensitivities >30% at 86.4% specificity.

Gender was included in the algorithm as an additional term to the biomarker terms (comprising intercept value and coefficient-weighted biomarker concentration values) in the linear equation. In the gender term, a base value 0 was applied for maleness and 1 for femaleness, weighted with its own coefficient value. It will be apparent to those skilled in the art that base values could alternatively be 1 for maleness and 0 for femaleness without altering the generality of the approach.

In respect of Tables 21 to 28, there were no single biomarkers which, in conjunction with gender, produced a sensitivity for advanced adenoma >30% at 86.4% specificity. XV=cross-validated; non XV=non cross-validated.

TABLE 21(a)
Two biomarker combinations plus gender having >30% sensitivity at 86.4% specificity. Combinations
also showing a ten-fold cross validated sensitivity of >30% at 86.4% specificity are shown in boldface.

| | | Sensitivity at 86.5% | | Sensitivity at 90% | | Sensitivity at 95% | |
|---|---|---|---|---|---|---|---|
| | | Specificity | | Specificity | | Specificity | |
| Biomarkers | | Non XV | XV | Non XV | XV | Non XV | XV |
| IGFBP2 | TIMP1 | 41.51% | 30.19% | 32.08% | 20.75% | 9.43% | 7.55% |
| IGFBP2 | IL13 | 35.85% | 32.08% | 22.64% | 18.87% | 15.09% | 5.66% |
| IGFBP2 | Mac2BP | 30.19% | 26.42% | 26.42% | 22.64% | 20.75% | 15.09% |
| IGFBP2 | M2PK | 30.19% | 26.42% | 24.53% | 16.98% | 11.32% | 13.21% |
| IGFBP2 | IL8 | 30.19% | 22.64% | 26.42% | 18.87% | 7.55% | 3.77% |
| IGFBP2 | EpCAM | 30.19% | 22.64% | 18.87% | 16.98% | 11.32% | 7.55% |
| IGFBP2 | TGFbeta1 | 30.19% | 22.64% | 22.64% | 16.98% | 7.55% | 5.66% |

TABLE 21(b)
Two biomarker, ten-fold cross validated combinations plus
gender having >30% sensitivity at 86.4% specificity

| | | Sensitivity | Sensitivity | Sensitivity |
|---|---|---|---|---|
| | | at 86.4% | at 90% | at 95% |
| | | specificity | specificity | specificity |
| | | (cross- | (cross- | (cross- |
| Biomarkers | | validated) | validated) | validated) |
| IGFBP2 | TIMP1 | 30.19 | 20.75 | 7.5 |
| IGFBP2 | IL-13 | 32.1 | 18.9 | 3.8 |

TABLE 22(a)
Three biomarker combinations plus gender having >30% sensitivity
at 86.4% specificity. Combinations also showing a ten-fold cross
validated sensitivity of >30% at 86.4% specificity are shown in boldface.

| | | | Sensitivity at 86.5% | | Sensitivity at 90% | | Sensitivity at 95% | |
|---|---|---|---|---|---|---|---|---|
| | | | Specificity | | Specificity | | Specificity | |
| Biomarkers | | | Non XV | XV | Non XV | XV | Non XV | XV |
| IGFBP2 | Mac2BP | TIMP1 | 43.40% | 33.96% | 37.74% | 30.19% | 16.98% | 9.43% |
| IGFBP2 | Dkk3 | Mac2BP | 41.51% | 28.30% | 24.53% | 18.87% | 20.75% | 16.98% |
| IGFBP2 | Dkk3 | TIMP1 | 39.62% | 24.53% | 30.19% | 18.87% | 9.43% | 5.66% |
| IGFBP2 | Mac2BP | TGFbeta | 37.74% | 32.08% | 35.85% | 20.75% | 18.87% | 15.09% |
| IGFBP2 | Mac2BP | IL13 | 37.74% | 30.19% | 33.96% | 24.53% | 26.42% | 9.43% |
| IGFBP2 | TGFbeta | TIMP1 | 37.74% | 28.30% | 33.96% | 22.64% | 9.43% | 3.77% |
| IGFBP2 | TIMP1 | IL13 | 37.74% | 28.30% | 28.30% | 20.75% | 11.32% | 9.43% |
| IGFBP2 | Mac2BP | EpCAM | 37.74% | 24.53% | 24.53% | 16.98% | 18.87% | 13.21% |
| IGFBP2 | IL8 | IL13 | 35.85% | 33.96% | 28.30% | 16.98% | 9.43% | 3.77% |
| IGFBP2 | M2PK | Mac2BP | 35.85% | 28.30% | 30.19% | 22.64% | 20.75% | 16.98% |
| IGFBP2 | TIMP1 | IL8 | 35.85% | 26.42% | 30.19% | 18.87% | 11.32% | 7.55% |
| IGFBP2 | M2PK | TIMP1 | 35.85% | 24.53% | 26.42% | 16.98% | 13.21% | 7.55% |
| IGFBP2 | IL13 | EpCAM | 33.96% | 33.96% | 22.64% | 13.21% | 18.87% | 5.66% |
| IGFBP2 | M2PK | IL13 | 33.96% | 28.30% | 24.53% | 18.87% | 15.09% | 7.55% |
| IGFBP2 | IL8 | EpCAM | 33.96% | 26.42% | 26.42% | 18.87% | 5.66% | 3.77% |
| IGFBP2 | TGFbeta | IL13 | 33.96% | 24.53% | 20.75% | 20.75% | 15.09% | 3.77% |
| IGFBP2 | Dkk3 | IL8 | 33.96% | 20.75% | 26.42% | 16.98% | 5.66% | 3.77% |
| IGFBP2 | TIMP1 | EpCAM | 32.08% | 28.30% | 32.08% | 20.75% | 13.21% | 11.32% |
| IGFBP2 | Dkk3 | TGFbeta | 32.08% | 22.64% | 22.64% | 16.98% | 1.89% | 0.00% |
| IGFBP2 | TGFbeta | IL8 | 32.08% | 22.64% | 26.42% | 15.09% | 7.55% | 3.77% |
| IGFBP2 | M2PK | EpCAM | 32.08% | 20.75% | 22.64% | 13.21% | 13.21% | 11.32% |
| IGFBP2 | Dkk3 | IL13 | 30.19% | 33.96% | 20.75% | 15.09% | 16.98% | 3.77% |
| IGFBP2 | M2PK | TGFbeta | 30.19% | 24.53% | 24.53% | 15.09% | 9.43% | 7.55% |
| IGFBP2 | Mac2BP | IL8 | 30.19% | 20.75% | 28.30% | 20.75% | 16.98% | 16.98% |
| IGFBP2 | M2PK | IL8 | 30.19% | 20.75% | 20.75% | 13.21% | 9.43% | 5.66% |

TABLE 22(b)
Three biomarker, 10-fold cross validated combinations
plus gender having >30% sensitivity at 86.4% specificity.

| | | | Sensitivity | Sensitivity | Sensitivity |
|---|---|---|---|---|---|
| | | | at 86.4% | at 90% | at 95% |
| | | | specificity | specificity | specificity |
| | | | cross- | (cross- | (cross- |
| Biomarkers | | | validated | validated) | validated) |
| IGFBP2 | Mac2BP | TIMP1 | 34.0 | 30.2 | 9.4 |
| IGFBP2 | Mac2BP | IL-13 | 30.19 | 24.5 | 3.8 |
| IGFBP2 | Mac2BP | TGFbeta | 32.01 | 20.7 | 15.1 |
| IGFBP2 | IL-8 | IL-13 | 34.0 | 17.0 | 3.8 |
| IGFBP2 | DKK-3 | IL-13 | 34.0 | 15.1 | 3.8 |
| IGFBP2 | IL-13 | EpCAM | 34.0 | 13.2 | 5.7 |

TABLE 23(a)
Four biomarker combinations plus gender having >30% sensitivity at
86.4% specificity. Combinations also showing a ten-fold cross
validated sensitivity of >30% at 86.4% specificity are
in boldface.

| | | | | Sensitivity at 86.5% | | Sensitivity at 90% | | Sensitivity at 95% | |
|---|---|---|---|---|---|---|---|---|---|
| | | | | Specificity | | Specificity | | Specificity | |
| Biomarkers | | | | Non XV | XV | Non XV | XV | Non XV | XV |
| IGFBP2 | M2PK | Mac2BP | TIMP1 | 45.28% | 32.08% | 33.96% | 24.53% | 20.75% | 15.09% |
| IGFBP2 | Mac2BP | TGFbeta | TIMP1 | 43.40% | 32.08% | 35.85% | 22.64% | 18.87% | 13.21% |
| IGFBP2 | Mac2BP | TIMP1 | IL8 | 43.40% | 28.30% | 33.96% | 20.75% | 20.75% | 11.32% |
| IGFBP2 | Dkk3 | Mac2BP | TIMP1 | 41.51% | 32.08% | 33.96% | 20.75% | 16.98% | 9.43% |
| IGFBP2 | M2PK | Mac2BP | TGFbeta | 41.51% | 28.30% | 32.08% | 18.87% | 18.87% | 16.98% |
| IGFBP2 | TIMP1 | IL13 | EpCAM | 39.62% | 32.08% | 32.08% | 18.87% | 11.32% | 7.55% |
| IGFBP2 | Mac2BP | IL8 | IL13 | 39.62% | 30.19% | 33.96% | 26.42% | 20.75% | 7.55% |
| IGFBP2 | Mac2BP | TGFbeta | IL13 | 39.62% | 30.19% | 32.08% | 20.75% | 26.42% | 7.55% |
| IGFBP2 | Dkk3 | M2PK | Mac2BP | 39.62% | 28.30% | 28.30% | 18.87% | 16.98% | 13.21% |
| IGFBP2 | M2PK | TIMP1 | IL8 | 39.62% | 28.30% | 24.53% | 16.98% | 9.43% | 7.55% |
| IGFBP2 | Dkk3 | Mac2BP | TGFbeta | 39.62% | 26.42% | 28.30% | 20.75% | 22.64% | 15.09% |
| IGFBP2 | Dkk3 | TIMP1 | IL13 | 39.62% | 26.42% | 28.30% | 16.98% | 15.09% | 5.66% |
| IGFBP2 | Dkk3 | TIMP1 | IL8 | 39.62% | 24.53% | 30.19% | 16.98% | 11.32% | 7.55% |
| IGFBP2 | Dkk3 | Mac2BP | IL13 | 37.74% | 33.96% | 30.19% | 15.09% | 15.09% | 7.55% |
| IGFBP2 | M2PK | Mac2BP | IL13 | 37.74% | 32.08% | 32.08% | 22.64% | 16.98% | 11.32% |
| IGFBP2 | TIMP1 | IL8 | IL13 | 37.74% | 32.08% | 32.08% | 20.75% | 11.32% | 3.77% |
| IGFBP2 | M2PK | TIMP1 | IL13 | 37.74% | 30.19% | 26.42% | 22.64% | 13.21% | 7.55% |
| IGFBP2 | Mac2BP | TIMP1 | IL13 | 37.74% | 30.19% | 37.74% | 20.75% | 18.87% | 11.32% |
| IGFBP2 | Mac2BP | IL13 | EpCAM | 37.74% | 28.30% | 30.19% | 16.98% | 24.53% | 11.32% |
| IGFBP2 | M2PK | Mac2BP | EpCAM | 37.74% | 24.53% | 30.19% | 20.75% | 22.64% | 16.98% |
| IGFBP2 | TGFbeta | TIMP1 | IL13 | 37.74% | 24.53% | 30.19% | 20.75% | 11.32% | 3.77% |
| IGFBP2 | TGFbeta | TIMP1 | IL8 | 37.74% | 24.53% | 30.19% | 20.75% | 11.32% | 3.77% |
| IGFBP2 | M2PK | TGFbeta | TIMP1 | 37.74% | 24.53% | 20.75% | 18.87% | 11.32% | 5.66% |
| IGFBP2 | Dkk3 | TGFbeta | TIMP1 | 37.74% | 24.53% | 26.42% | 16.98% | 11.32% | 3.77% |
| IGFBP2 | Dkk3 | M2PK | TIMP1 | 37.74% | 16.98% | 24.53% | 16.98% | 13.21% | 5.66% |
| IGFBP2 | IL8 | IL13 | EpCAM | 35.85% | 32.08% | 28.30% | 20.75% | 9.43% | 3.77% |
| IGFBP2 | Dkk3 | IL8 | IL13 | 35.85% | 32.08% | 28.30% | 20.75% | 7.55% | 1.89% |
| IGFBP2 | Mac2BP | TGFbeta | EpCAM | 35.85% | 30.19% | 32.08% | 22.64% | 18.87% | 13.21% |
| IGFBP2 | TGFbeta | IL8 | IL13 | 35.85% | 30.19% | 22.64% | 11.32% | 9.43% | 5.66% |
| IGFBP2 | Dkk3 | Mac2BP | EpCAM | 35.85% | 28.30% | 30.19% | 24.53% | 20.75% | 13.21% |
| IGFBP2 | Mac2BP | TGFbeta | IL8 | 35.85% | 28.30% | 24.53% | 18.87% | 22.64% | 16.98% |
| IGFBP2 | M2PK | TIMP1 | EpCAM | 35.85% | 26.42% | 32.08% | 20.75% | 9.43% | 9.43% |
| IGFBP2 | Mac2BP | IL8 | EpCAM | 35.85% | 22.64% | 24.53% | 20.75% | 18.87% | 13.21% |
| IGFBP2 | Dkk3 | M2PK | IL8 | 35.85% | 22.64% | 18.87% | 15.09% | 7.55% | 5.66% |
| IGFBP2 | Dkk3 | Mac2BP | IL8 | 35.85% | 20.75% | 33.96% | 18.87% | 15.09% | 9.43% |
| IGFBP2 | TGFbeta | IL13 | EpCAM | 33.96% | 28.30% | 20.75% | 16.98% | 15.09% | 5.66% |
| IGFBP2 | M2PK | IL8 | IL13 | 33.96% | 28.30% | 24.53% | 16.98% | 11.32% | 7.55% |
| IGFBP2 | Mac2BP | TIMP1 | EpCAM | 33.96% | 26.42% | 32.08% | 26.42% | 24.53% | 13.21% |
| IGFBP2 | M2PK | Mac2BP | IL8 | 33.96% | 26.42% | 33.96% | 24.53% | 15.09% | 11.32% |
| IGFBP2 | M2PK | TGFbeta | IL13 | 33.96% | 26.42% | 24.53% | 18.87% | 13.21% | 9.43% |
| IGFBP2 | TIMP1 | IL8 | EpCAM | 33.96% | 24.53% | 32.08% | 18.87% | 11.32% | 9.43% |
| IGFBP2 | Dkk3 | TIMP1 | EpCAM | 33.96% | 22.64% | 26.42% | 18.87% | 11.32% | 7.55% |
| IGFBP2 | TGFbeta | TIMP1 | EpCAM | 32.08% | 26.42% | 30.19% | 20.75% | 16.98% | 5.66% |
| IGFBP2 | Dkk3 | IL8 | EpCAM | 32.08% | 24.53% | 26.42% | 16.98% | 3.77% | 1.89% |
| IGFBP2 | Dkk3 | TGFbeta | IL13 | 32.08% | 22.64% | 20.75% | 13.21% | 15.09% | 5.66% |
| IGFBP2 | Dkk3 | TGFbeta | IL8 | 32.08% | 22.64% | 26.42% | 13.21% | 3.77% | 3.77% |
| IGFBP2 | M2PK | IL8 | EpCAM | 32.08% | 20.75% | 24.53% | 18.87% | 7.55% | 5.66% |
| IGFBP2 | M2PK | TGFbeta | IL8 | 32.08% | 20.75% | 18.87% | 18.87% | 5.66% | 3.77% |
| IGFBP2 | Dkk3 | IL13 | EpCAM | 30.19% | 33.96% | 20.75% | 13.21% | 16.98% | 3.77% |
| IGFBP2 | Dkk3 | M2PK | IL13 | 30.19% | 28.30% | 22.64% | 15.09% | 13.21% | 1.89% |
| IGFBP2 | M2PK | IL13 | EpCAM | 30.19% | 28.30% | 24.53% | 13.21% | 13.21% | 7.55% |
| IGFBP2 | Dkk3 | TGFbeta | EpCAM | 30.19% | 24.53% | 24.53% | 15.09% | 9.43% | 1.89% |
| IGFBP2 | Dkk3 | M2PK | TGFbeta | 30.19% | 22.64% | 24.53% | 13.21% | 7.55% | 3.77% |
| M2PK | TIMP1 | IL8 | IL13 | 30.19% | 16.98% | 16.98% | 11.32% | 11.32% | 9.43% |

TABLE 23(b)
Four biomarker, ten-fold cross validated combinations plus gender
having >30% sensitivity at 86.4% specificity.

| | | | | Sensitivity at 86.4% | Sensitivity at 90% | Sensitivity at 95% |
|---|---|---|---|---|---|---|
| | | | | specificity | specificity (cross- | specificity (cross- |
| Biomarkers | | | | cross-validated | validated) | validated) |
| IGFBP2 | Mac2BP | IL-8 | IL-13 | 30.19 | 26.4 | 7.6 |
| IGFBP2 | Mac2BP | M2PK | TIMP1 | 32.01 | 24.5 | 15.1 |
| IGFBP2 | Mac2BP | TGFbeta | EpCAM | 30.19 | 22.6 | 13.2 |
| IGFBP2 | Mac2BP | M2PK | IL-13 | 32.08 | 22.6 | 11.3 |
| IGFBP2 | Mac2BP | TGFbeta | TIMP1 | 32.08 | 22.6 | 13.2 |
| IGFBP2 | IL-8 | IL-13 | EpCAM | 32.08 | 20.7 | 3.8 |
| IGFBP2 | IL-8 | IL-13 | TIMP1 | 32.08 | 20.7 | 3.8 |
| IGFBP2 | IL-8 | IL-13 | DKK3 | 32.08 | 20.7 | 18.9 |
| IGFBP2 | Mac2BP | IL-13 | TIMP1 | 30.19 | 20.7 | 11.3 |
| IGFBP2 | Mac2BP | TGFbeta | IL-13 | 30.19 | 20.7 | 7.6 |
| IGFBP2 | Mac2BP | DKK3 | TIMP1 | 32.08 | 20.7 | 9.4 |
| IGFBP2 | EpCAM | IL-13 | TIMP1 | 32.08 | 18.9 | 7.6 |
| IGFBP2 | Mac2BP | IL-13 | DKK3 | 34.0 | 15.1 | 7.6 |
| IGFBP2 | EpCAM | IL-13 | DKK3 | 34.0 | 13.2 | 3.8 |
| IGFBP2 | TGFbeta | IL-13 | IL-8 | 30.19 | 11.3 | 5.7 |
| IGFBP2 | M2PK | TIMP1 | IL-13 | 30.19 | 22.6 | 7.6 |

TABLE 24(a)
Five biomarker combinations plus gender having >30% sensitivity at
86.4% specificity. Combinations also showing a ten-fold cross
validated sensitivity of >30% at 86.4% specificity are shown in boldface.

| | | | | | Sensitivity at 86.4% | | Sensitivity at 90% | | Sensitivity at 95% | |
|---|---|---|---|---|---|---|---|---|---|---|
| | | | | | Specificity | | Specificity | | Specificity | |
| Biomarkers | | | | | Non XV | XV | Non XV | XV | Non XV | XV |
| IGFBP2 | Dkk3 | M2PK | Mac2BP | TIMP1 | 45.28% | 28.30% | 33.96% | 20.75% | 18.87% | 11.32% |
| IGFBP2 | M2PK | Mac2BP | TGFbeta | TIMP1 | 43.40% | 32.08% | 35.85% | 20.75% | 20.75% | 11.32% |
| IGFBP2 | Dkk3 | Mac2BP | TIMP1 | IL8 | 41.51% | 30.19% | 32.08% | 20.75% | 15.09% | 11.32% |
| IGFBP2 | Mac2BP | TGFbeta | IL8 | IL13 | 41.51% | 28.30% | 35.85% | 24.53% | 18.87% | 5.66% |
| IGFBP2 | M2PK | Mac2BP | TIMP1 | IL8 | 41.51% | 26.42% | 28.30% | 18.87% | 20.75% | 13.21% |
| IGFBP2 | Dkk3 | Mac2BP | TGFbeta | TIMP1 | 41.51% | 24.53% | 32.08% | 20.75% | 16.98% | 9.43% |
| IGFBP2 | TIMP1 | IL8 | IL13 | EpCAM | 39.62% | 33.96% | 33.96% | 18.87% | 11.32% | 3.77% |
| IGFBP2 | Dkk3 | Mac2BP | IL8 | IL13 | 39.62% | 32.08% | 37.74% | 24.53% | 20.75% | 5.66% |
| IGFBP2 | Dkk3 | M2PK | Mac2BP | IL13 | 39.62% | 32.08% | 30.19% | 18.87% | 18.87% | 7.55% |
| IGFBP2 | Mac2BP | TGFbeta | TIMP1 | IL8 | 39.62% | 30.19% | 33.96% | 22.64% | 20.75% | 11.32% |
| IGFBP2 | Dkk3 | Mac2BP | TIMP1 | IL13 | 39.62% | 30.19% | 37.74% | 20.75% | 15.09% | 7.55% |
| IGFBP2 | Mac2BP | TIMP1 | IL13 | EpCAM | 39.62% | 30.19% | 35.85% | 16.98% | 16.98% | 11.32% |
| IGFBP2 | M2PK | Mac2BP | IL8 | IL13 | 39.62% | 28.30% | 35.85% | 26.42% | 15.09% | 7.55% |
| IGFBP2 | Dkk3 | TIMP1 | IL13 | EpCAM | 39.62% | 28.30% | 30.19% | 16.98% | 15.09% | 9.43% |
| IGFBP2 | Mac2BP | TIMP1 | IL8 | IL13 | 39.62% | 26.42% | 39.62% | 26.42% | 13.21% | 7.55% |
| IGFBP2 | M2PK | Mac2BP | TIMP1 | IL13 | 39.62% | 26.42% | 32.08% | 24.53% | 22.64% | 13.21% |
| IGFBP2 | Mac2BP | TGFbeta | TIMP1 | IL13 | 39.62% | 26.42% | 37.74% | 20.75% | 16.98% | 9.43% |
| IGFBP2 | M2PK | TGFbeta | TIMP1 | IL8 | 39.62% | 26.42% | 24.53% | 20.75% | 11.32% | 5.66% |
| IGFBP2 | M2PK | TGFbeta | IL8 | IL13 | 39.62% | 26.42% | 24.53% | 18.87% | 9.43% | 1.89% |
| IGFBP2 | Dkk3 | M2PK | Mac2BP | TGFbeta | 39.62% | 26.42% | 30.19% | 16.98% | 18.87% | 15.09% |
| IGFBP2 | TGFbeta | TIMP1 | IL13 | EpCAM | 39.62% | 24.53% | 28.30% | 16.98% | 9.43% | 5.66% |
| IGFBP2 | Dkk3 | M2PK | Mac2BP | EpCAM | 39.62% | 20.75% | 30.19% | 18.87% | 18.87% | 11.32% |
| IGFBP2 | Dkk3 | TGFbeta | TIMP1 | IL13 | 39.62% | 20.75% | 28.30% | 16.98% | 11.32% | 3.77% |
| IGFBP2 | Dkk3 | TGFbeta | TIMP1 | IL8 | 39.62% | 20.75% | 30.19% | 16.98% | 9.43% | 3.77% |
| IGFBP2 | Dkk3 | TIMP1 | IL8 | IL13 | 37.74% | 32.08% | 33.96% | 16.98% | 13.21% | 3.77% |
| IGFBP2 | Mac2BP | IL8 | IL13 | EpCAM | 37.74% | 30.19% | 37.74% | 28.30% | 22.64% | 7.55% |
| IGFBP2 | M2PK | TIMP1 | IL13 | EpCAM | 37.74% | 30.19% | 32.08% | 24.53% | 9.43% | 7.55% |
| IGFBP2 | M2PK | Mac2BP | TGFbeta | IL13 | 37.74% | 30.19% | 32.08% | 24.53% | 22.64% | 9.43% |
| IGFBP2 | M2PK | TIMP1 | IL8 | IL13 | 37.74% | 30.19% | 30.19% | 22.64% | 9.43% | 3.77% |
| IGFBP2 | M2PK | TGFbeta | TIMP1 | IL13 | 37.74% | 30.19% | 26.42% | 20.75% | 11.32% | 7.55% |
| IGFBP2 | Dkk3 | M2PK | TIMP1 | IL13 | 37.74% | 28.30% | 28.30% | 20.75% | 13.21% | 7.55% |
| IGFBP2 | M2PK | Mac2BP | TGFbeta | IL8 | 37.74% | 28.30% | 35.85% | 20.75% | 20.75% | 11.32% |
| IGFBP2 | Dkk3 | Mac2BP | TGFbeta | IL13 | 37.74% | 28.30% | 32.08% | 15.09% | 18.87% | 7.55% |
| IGFBP2 | Dkk3 | Mac2BP | IL13 | EpCAM | 37.74% | 28.30% | 30.19% | 13.21% | 15.09% | 7.55% |
| IGFBP2 | Dkk3 | M2PK | Mac2BP | IL8 | 37.74% | 26.42% | 32.08% | 20.75% | 15.09% | 9.43% |
| IGFBP2 | Mac2BP | TGFbeta | IL8 | EpCAM | 37.74% | 24.53% | 30.19% | 24.53% | 18.87% | 13.21% |
| IGFBP2 | Dkk3 | M2PK | TIMP1 | IL8 | 37.74% | 24.53% | 32.08% | 13.21% | 9.43% | 5.66% |
| IGFBP2 | Dkk3 | TIMP1 | IL8 | EpCAM | 37.74% | 20.75% | 32.08% | 18.87% | 11.32% | 7.55% |
| IGFBP2 | Dkk3 | M2PK | TGFbeta | TIMP1 | 37.74% | 16.98% | 24.53% | 13.21% | 13.21% | 3.77% |
| IGFBP2 | TGFbeta | TIMP1 | IL8 | IL13 | 35.85% | 30.19% | 35.85% | 26.42% | 11.32% | 3.77% |
| IGFBP2 | Dkk3 | IL8 | IL13 | EpCAM | 35.85% | 30.19% | 28.30% | 22.64% | 7.55% | 1.89% |
| IGFBP2 | M2PK | Mac2BP | IL13 | EpCAM | 35.85% | 30.19% | 33.96% | 20.75% | 16.98% | 11.32% |
| IGFBP2 | TGFbeta | IL8 | IL13 | EpCAM | 35.85% | 28.30% | 26.42% | 11.32% | 9.43% | 3.77% |
| IGFBP2 | M2PK | Mac2BP | TIMP1 | EpCAM | 35.85% | 26.42% | 33.96% | 26.42% | 22.64% | 16.98% |
| IGFBP2 | M2PK | Mac2BP | TGFbeta | EpCAM | 35.85% | 26.42% | 35.85% | 22.64% | 22.64% | 15.09% |
| IGFBP2 | Dkk3 | TGFbeta | IL8 | IL13 | 35.85% | 26.42% | 28.30% | 11.32% | 9.43% | 1.89% |
| IGFBP2 | M2PK | TGFbeta | TIMP1 | EpCAM | 35.85% | 24.53% | 30.19% | 20.75% | 15.09% | 9.43% |
| IGFBP2 | Dkk3 | Mac2BP | IL8 | EpCAM | 35.85% | 24.53% | 33.96% | 18.87% | 15.09% | 9.43% |
| IGFBP2 | Mac2BP | TGFbeta | IL13 | EpCAM | 35.85% | 24.53% | 30.19% | 16.98% | 26.42% | 13.21% |
| IGFBP2 | Dkk3 | M2PK | TGFbeta | IL8 | 35.85% | 24.53% | 32.08% | 13.21% | 5.66% | 3.77% |
| IGFBP2 | Dkk3 | Mac2BP | TGFbeta | IL8 | 35.85% | 22.64% | 33.96% | 20.75% | 20.75% | 11.32% |
| IGFBP2 | Dkk3 | TGFbeta | TIMP1 | EpCAM | 35.85% | 22.64% | 28.30% | 18.87% | 15.09% | 7.55% |
| IGFBP2 | Dkk3 | Mac2BP | TIMP1 | EpCAM | 35.85% | 20.75% | 32.08% | 20.75% | 26.42% | 11.32% |
| IGFBP2 | Dkk3 | M2PK | TIMP1 | EpCAM | 35.85% | 16.98% | 32.08% | 16.98% | 9.43% | 7.55% |
| IGFBP2 | M2PK | IL8 | IL13 | EpCAM | 33.96% | 30.19% | 24.53% | 18.87% | 13.21% | 7.55% |
| IGFBP2 | Mac2BP | TIMP1 | IL8 | EpCAM | 33.96% | 28.30% | 32.08% | 20.75% | 18.87% | 15.09% |
| IGFBP2 | Dkk3 | Mac2BP | TGFbeta | EpCAM | 33.96% | 26.42% | 30.19% | 22.64% | 22.64% | 11.32% |
| IGFBP2 | Dkk3 | M2PK | IL8 | IL13 | 33.96% | 26.42% | 24.53% | 18.87% | 13.21% | 7.55% |
| IGFBP2 | Dkk3 | M2PK | TGFbeta | IL13 | 33.96% | 26.42% | 22.64% | 15.09% | 15.09% | 7.55% |
| IGFBP2 | M2PK | TIMP1 | IL8 | EpCAM | 33.96% | 24.53% | 33.96% | 20.75% | 9.43% | 7.55% |
| IGFBP2 | M2PK | TGFbeta | IL13 | EpCAM | 33.96% | 24.53% | 26.42% | 16.98% | 13.21% | 9.43% |
| IGFBP2 | M2PK | Mac2BP | IL8 | EpCAM | 33.96% | 22.64% | 32.08% | 20.75% | 18.87% | 11.32% |
| IGFBP2 | TGFbeta | TIMP1 | IL8 | EpCAM | 33.96% | 22.64% | 32.08% | 18.87% | 13.21% | 9.43% |
| IGFBP2 | Dkk3 | TGFbeta | IL13 | EpCAM | 33.96% | 22.64% | 22.64% | 11.32% | 15.09% | 5.66% |
| IGFBP2 | Dkk3 | M2PK | IL8 | EpCAM | 33.96% | 18.87% | 22.64% | 15.09% | 7.55% | 5.66% |
| IGFBP2 | Mac2BP | TGFbeta | TIMP1 | EpCAM | 32.08% | 26.42% | 32.08% | 26.42% | 24.53% | 11.32% |
| IGFBP2 | Dkk3 | M2PK | IL13 | EpCAM | 30.19% | 26.42% | 22.64% | 16.98% | 15.09% | 1.89% |
| IGFBP2 | Dkk3 | TGFbeta | IL8 | EpCAM | 30.19% | 24.53% | 28.30% | 15.09% | 7.55% | 1.89% |
| IGFBP2 | M2PK | TGFbeta | IL8 | EpCAM | 30.19% | 22.64% | 26.42% | 18.87% | 9.43% | 1.89% |
| IGFBP2 | Dkk3 | M2PK | TGFbeta | EpCAM | 30.19% | 20.75% | 24.53% | 13.21% | 7.55% | 3.77% |

TABLE 24(b)
Five biomarker, ten-fold cross validated combinations plus gender having >30% sensitivity at 86.4% specificity.

| | | | | | Sensitivity at 86.4% | Sensitivity at 90% | Sensitivity at 95% |
|---|---|---|---|---|---|---|---|
| | | | | | specificity cross- | specificity (cross- | specificity (cross- |
| Biomarkers | | | | | validated | validated) | validated) |
| IGFBP2 | Mac2BP | IL-8 | IL-13 | EpCAM | 30.19 | 28.3 | 7.6 |
| IGFBP2 | TGFbeta | IL-8 | IL-13 | TIMP1 | 30.19 | 26.4 | 3.8 |
| IGFBP2 | M2PK | EpCAM | IL-13 | TIMP1 | 30.19 | 24.5 | 7.6 |
| IGFBP2 | Mac2BP | IL-8 | IL-13 | DKK3 | 32.08 | 24.5 | 5.7 |
| IGFBP2 | Mac2BP | M2PK | IL-13 | TGFbeta | 30.19 | 24.5 | 9.4 |
| IGFBP2 | DKK3 | IL-8 | IL-13 | EpCAM | 30.19 | 22.6 | 19.0 |
| IGFBP2 | M2PK | IL-8 | IL-13 | TIMP1 | 30.19 | 22.6 | 3.8 |
| IGFBP2 | Mac2BP | IL-8 | TGFbeta | TIMP1 | 30.19 | 22.6 | 11.3 |
| IGFBP2 | Mac2BP | M2PK | IL-13 | EpCAM | 30.19 | 20.8 | 11.3 |
| IGFBP2 | M2PK | TGFbeta | IL-13 | TIMP1 | 30.19 | 20.6 | 7.6 |
| IGFBP2 | Mac2BP | DKK3 | IL-13 | TIMP1 | 30.19 | 20.8 | 7.6 |
| IGFBP2 | Mac2BP | DKK3 | IL-8 | TIMP1 | 30.19 | 20.6 | 11.3 |
| IGFBP2 | Mac2BP | M2PK | TGFbeta | TIMP1 | 32.08 | 20.6 | 11.3 |
| IGFBP2 | EpCAM | IL-8 | IL-13 | TIMP1 | 34.0 | 18.9 | 3.8 |
| IGFBP2 | M2PK | IL-8 | IL-13 | EpCAM | 30.19 | 18.9 | 7.6 |
| IGFBP2 | Mac2BP | M2PK | DKK3 | IL-13 | 32.08 | 18.9 | 7.6 |
| IGFBP2 | Mac2BP | TIMP1 | IL-13 | EpCAM | 30.19 | 17.0 | 11.3 |
| IGFBP2 | DKK3 | IL-8 | IL-13 | TIMP1 | 32.08 | 17.0 | 3.8 |

TABLE 25(a)
Six biomarker combinations plus gender having >30% sensitivity at
86.4% specificity. Combinations also showing a ten-fold cross
validated sensitivity of >30% at 86.4% specificity are shown in boldface.

| | | | | | | Sensitivity at 86.4% | | Sensitivity at 90% | | Sensitivity at 95% | |
|---|---|---|---|---|---|---|---|---|---|---|---|
| | | | | | | Specificity | | Specificity | | Specificity | |
| Biomarkers | | | | | | Non XV | XV | Non XV | XV | Non XV | XV |
| IGFBP2 | M2PK | Mac2BP | TGFbeta | TIMP1 | IL8 | 45.28% | 26.42% | 32.08% | 18.87% | 22.64% | 13.21% |
| IGFBP2 | Dkk3 | M2PK | Mac2BP | TGFbeta | TIMP1 | 45.28% | 24.53% | 39.62% | 16.98% | 16.98% | 11.32% |
| IGFBP2 | Dkk3 | M2PK | Mac2BP | TIMP1 | IL8 | 43.40% | 30.19% | 37.74% | 15.09% | 16.98% | 13.21% |
| IGFBP2 | Mac2BP | TIMP1 | IL8 | IL13 | EpCAM | 41.51% | 32.08% | 39.62% | 24.53% | 11.32% | 5.66% |
| IGFBP2 | Mac2BP | TGFbeta | TIMP1 | IL8 | IL13 | 41.51% | 28.30% | 37.74% | 24.53% | 13.21% | 5.66% |
| IGFBP2 | Mac2BP | TGFbeta | IL8 | IL13 | EpCAM | 41.51% | 26.42% | 37.74% | 26.42% | 18.87% | 7.55% |
| IGFBP2 | Dkk3 | Mac2BP | TGFbeta | TIMP1 | IL8 | 41.51% | 26.42% | 32.08% | 20.75% | 16.98% | 9.43% |
| IGFBP2 | Mac2BP | TGFbeta | TIMP1 | IL13 | EpCAM | 41.51% | 24.53% | 33.96% | 13.21% | 16.98% | 9.43% |
| IGFBP2 | Dkk3 | Mac2BP | IL8 | IL13 | EpCAM | 39.62% | 32.08% | 37.74% | 28.30% | 18.87% | 5.66% |
| IGFBP2 | Dkk3 | Mac2BP | TIMP1 | IL8 | IL13 | 39.62% | 32.08% | 39.62% | 24.53% | 15.09% | 3.77% |
| IGFBP2 | Dkk3 | M2PK | Mac2BP | IL8 | IL13 | 39.62% | 30.19% | 37.74% | 26.42% | 16.98% | 7.55% |
| IGFBP2 | M2PK | Mac2BP | TIMP1 | IL8 | IL13 | 39.62% | 28.30% | 32.08% | 24.53% | 15.09% | 11.32% |
| IGFBP2 | M2PK | Mac2BP | TGFbeta | IL8 | IL13 | 39.62% | 28.30% | 37.74% | 22.64% | 20.75% | 11.32% |
| IGFBP2 | Dkk3 | M2PK | Mac2BP | TIMP1 | IL13 | 39.62% | 28.30% | 30.19% | 20.75% | 20.75% | 9.43% |
| IGFBP2 | Dkk3 | TIMP1 | IL8 | IL13 | EpCAM | 39.62% | 28.30% | 33.96% | 16.98% | 13.21% | 1.89% |
| IGFBP2 | M2PK | Mac2BP | IL8 | IL13 | EpCAM | 39.62% | 26.42% | 35.85% | 24.53% | 15.09% | 5.66% |
| IGFBP2 | Dkk3 | Mac2BP | TGFbeta | IL8 | IL13 | 39.62% | 26.42% | 35.85% | 24.53% | 18.87% | 3.77% |
| IGFBP2 | Dkk3 | M2PK | Mac2BP | TGFbeta | IL13 | 39.62% | 26.42% | 28.30% | 22.64% | 20.75% | 7.55% |
| IGFBP2 | Dkk3 | Mac2BP | TIMP1 | IL13 | EpCAM | 39.62% | 26.42% | 37.74% | 16.98% | 13.21% | 9.43% |
| IGFBP2 | Dkk3 | M2PK | Mac2BP | IL13 | EpCAM | 39.62% | 24.53% | 28.30% | 16.98% | 20.75% | 7.55% |
| IGFBP2 | Dkk3 | M2PK | Mac2BP | TGFbeta | IL8 | 39.62% | 22.64% | 33.96% | 20.75% | 16.98% | 11.32% |
| IGFBP2 | M2PK | TGFbeta | IL8 | IL13 | EpCAM | 39.62% | 22.64% | 24.53% | 16.98% | 9.43% | 1.89% |
| IGFBP2 | M2PK | Mac2BP | TGFbeta | IL13 | EpCAM | 39.62% | 20.75% | 33.96% | 18.87% | 22.64% | 9.43% |
| IGFBP2 | Dkk3 | TGFbeta | TIMP1 | IL13 | EpCAM | 39.62% | 20.75% | 30.19% | 16.98% | 11.32% | 7.55% |
| IGFBP2 | Dkk3 | Mac2BP | TGFbeta | IL13 | EpCAM | 39.62% | 20.75% | 30.19% | 15.09% | 18.87% | 7.55% |
| IGFBP2 | M2PK | TIMP1 | IL8 | IL13 | EpCAM | 37.74% | 28.30% | 32.08% | 22.64% | 9.43% | 3.77% |
| IGFBP2 | Dkk3 | M2PK | TIMP1 | IL13 | EpCAM | 37.74% | 28.30% | 28.30% | 18.87% | 13.21% | 5.66% |
| IGFBP2 | M2PK | Mac2BP | TGFbeta | TIMP1 | EpCAM | 37.74% | 26.42% | 33.96% | 26.42% | 24.53% | 13.21% |
| IGFBP2 | M2PK | Mac2BP | TIMP1 | IL8 | EpCAM | 37.74% | 26.42% | 37.74% | 24.53% | 20.75% | 13.21% |
| IGFBP2 | M2PK | Mac2BP | TGFbeta | TIMP1 | IL13 | 37.74% | 26.42% | 32.08% | 24.53% | 22.64% | 7.55% |
| IGFBP2 | Dkk3 | M2PK | TIMP1 | IL8 | IL13 | 37.74% | 26.42% | 32.08% | 20.75% | 9.43% | 3.77% |
| IGFBP2 | Dkk3 | M2PK | TGFbeta | TIMP1 | IL13 | 37.74% | 24.53% | 28.30% | 20.75% | 11.32% | 5.66% |
| IGFBP2 | Dkk3 | M2PK | TGFbeta | IL8 | IL13 | 37.74% | 24.53% | 28.30% | 16.98% | 9.43% | 1.89% |
| IGFBP2 | Dkk3 | Mac2BP | TGFbeta | TIMP1 | IL13 | 37.74% | 24.53% | 37.74% | 15.09% | 15.09% | 7.55% |
| IGFBP2 | Dkk3 | M2PK | TGFbeta | TIMP1 | IL8 | 37.74% | 24.53% | 32.08% | 13.21% | 9.43% | 5.66% |
| IGFBP2 | Dkk3 | M2PK | Mac2BP | TGFbeta | EpCAM | 37.74% | 20.75% | 33.96% | 20.75% | 18.87% | 13.21% |
| IGFBP2 | M2PK | TGFbeta | TIMP1 | IL13 | EpCAM | 35.85% | 32.08% | 28.30% | 16.98% | 9.43% | 7.55% |
| IGFBP2 | TGFbeta | TIMP1 | IL8 | IL13 | EpCAM | 35.85% | 30.19% | 35.85% | 22.64% | 11.32% | 3.77% |
| IGFBP2 | Dkk3 | TGFbeta | TIMP1 | IL8 | IL13 | 35.85% | 26.42% | 35.85% | 24.53% | 13.21% | 3.77% |
| IGFBP2 | M2PK | Mac2BP | TIMP1 | IL13 | EpCAM | 35.85% | 26.42% | 32.08% | 22.64% | 22.64% | 11.32% |
| IGFBP2 | M2PK | Mac2BP | TGFbeta | IL8 | EpCAM | 35.85% | 26.42% | 32.08% | 22.64% | 22.64% | 15.09% |
| IGFBP2 | Dkk3 | TGFbeta | IL8 | IL13 | EpCAM | 35.85% | 26.42% | 28.30% | 11.32% | 9.43% | 1.89% |
| IGFBP2 | Dkk3 | M2PK | Mac2BP | TIMP1 | EpCAM | 35.85% | 24.53% | 35.85% | 24.53% | 18.87% | 11.32% |
| IGFBP2 | Dkk3 | Mac2BP | TGFbeta | IL8 | EpCAM | 35.85% | 24.53% | 32.08% | 22.64% | 20.75% | 13.21% |
| IGFBP2 | M2PK | TGFbeta | TIMP1 | IL8 | IL13 | 35.85% | 24.53% | 28.30% | 22.64% | 9.43% | 7.55% |
| IGFBP2 | Dkk3 | Mac2BP | TIMP1 | IL8 | EpCAM | 35.85% | 24.53% | 33.96% | 20.75% | 22.64% | 11.32% |
| IGFBP2 | Dkk3 | M2PK | Mac2BP | IL8 | EpCAM | 35.85% | 22.64% | 33.96% | 18.87% | 15.09% | 9.43% |
| IGFBP2 | Dkk3 | Mac2BP | TGFbeta | TIMP1 | EpCAM | 35.85% | 20.75% | 32.08% | 20.75% | 26.42% | 11.32% |
| IGFBP2 | Dkk3 | M2PK | TGFbeta | TIMP1 | EpCAM | 35.85% | 16.98% | 32.08% | 15.09% | 9.43% | 7.55% |
| IGFBP2 | Mac2BP | TGFbeta | TIMP1 | IL8 | EpCAM | 33.96% | 28.30% | 32.08% | 22.64% | 24.53% | 11.32% |
| IGFBP2 | M2PK | TGFbeta | TIMP1 | IL8 | EpCAM | 33.96% | 24.53% | 33.96% | 20.75% | 11.32% | 9.43% |
| IGFBP2 | Dkk3 | M2PK | TGFbeta | IL13 | EpCAM | 33.96% | 22.64% | 22.64% | 13.21% | 15.09% | 7.55% |
| IGFBP2 | Dkk3 | M2PK | TIMP1 | IL8 | EpCAM | 33.96% | 20.75% | 33.96% | 18.87% | 7.55% | 7.55% |
| IGFBP2 | Dkk3 | TGFbeta | TIMP1 | IL8 | EpCAM | 33.96% | 18.87% | 28.30% | 18.87% | 11.32% | 5.66% |
| IGFBP2 | Dkk3 | M2PK | TGFbeta | IL8 | EpCAM | 33.96% | 16.98% | 26.42% | 16.98% | 5.66% | 3.77% |
| IGFBP2 | Dkk3 | M2PK | IL8 | IL13 | EpCAM | 30.19% | 24.53% | 26.42% | 18.87% | 13.21% | 7.55% |
| Dkk3 | M2PK | TGFbeta | TIMP1 | IL8 | IL13 | 30.19% | 16.98% | 18.87% | 9.43% | 13.21% | 5.66% |

TABLE 25(b)
Six biomarker, ten-fold cross validated combinations
plus gender having >30% sensitivity at 86.4% specificity

| | | | | | | Sensitivity at | Sensitivity at 90% | Sensitivity at 95% |
|---|---|---|---|---|---|---|---|---|
| | | | | | | 86.4% specificity | specificity (cross- | specificity (cross- |
| Biomarkers | | | | | | cross-validated | validated) | validated) |
| IGFBP2 | Mac2BP | M2PK | DKK3 | IL-8 | IL-13 | 30.19 | 26.4 | 7.6 |
| IGFBP2 | Mac2BP | TIMP1 | EpCAM | IL-8 | IL-13 | 32.08 | 24.5 | 5.7 |
| IGFBP2 | Mac2BP | TIMP1 | DKK3 | IL-8 | IL-13 | 32.08 | 24.5 | 3.8 |
| IGFBP2 | Mac2BP | Dkk3 | EpCAM | IL8 | IL13 | 32.08 | 28.0 | 5.7 |
| IGFBP2 | TGFbeta | TIMP1 | EpCAM | IL-8 | IL-13 | 30.19 | 22.6 | 3.8 |
| IGFBP2 | M2PK | TIMP1 | TGFbeta | EpCAM | IL-13 | 32.08 | 17.0 | 7.6 |
| IGFBP2 | Mac2BP | M2PK | TIMP1 | IL-8 | DKK3 | 30.19 | 15.1 | 13.2 |

TABLE 26(a)
Seven biomarker combinations plus gender having >30% sensitivity at
86.4% specificity. Combinations also showing a ten-fold cross
validated sensitivity of >30% at 86.4% specificity are shown in boldface.

| | | | | | | | Sensitivity at 86.4% | | Sensitivity at 90% | | Sensitivity at 95% | |
|---|---|---|---|---|---|---|---|---|---|---|---|---|
| | | | | | | | Specificity | | Specificity | | Specificity | |
| Biomarkers | | | | | | | Non XV | XV | Non XV | XV | Non XV | XV |
| IGFBP2 | M2PK | Mac2BP | TIMP1 | IL8 | IL13 | EpCAM | 43.40% | 26.42% | 32.08% | 24.53% | 15.09% | 9.43% |
| IGFBP2 | Dkk3 | M2PK | Mac2BP | TGFbeta | TIMP1 | IL8 | 43.40% | 26.42% | 39.62% | 16.98% | 18.87% | 13.21% |
| IGFBP2 | M2PK | Mac2BP | TGFbeta | IL8 | IL13 | EpCAM | 41.51% | 28.30% | 37.74% | 22.64% | 20.75% | 9.43% |
| IGFBP2 | Dkk3 | Mac2BP | TGFbeta | TIMP1 | IL8 | IL13 | 41.51% | 28.30% | 39.62% | 22.64% | 15.09% | 3.77% |
| IGFBP2 | Mac2BP | TGFbeta | TIMP1 | IL8 | IL13 | EpCAM | 41.51% | 28.30% | 37.74% | 20.75% | 11.32% | 5.66% |
| IGFBP2 | Dkk3 | M2PK | Mac2BP | TIMP1 | IL13 | EpCAM | 41.51% | 26.42% | 30.19% | 22.64% | 22.64% | 9.43% |
| IGFBP2 | Dkk3 | M2PK | Mac2BP | TIMP1 | IL8 | IL13 | 41.51% | 26.42% | 32.08% | 22.64% | 15.09% | 7.55% |
| IGFBP2 | Dkk3 | Mac2BP | TIMP1 | IL8 | IL13 | EpCAM | 39.62% | 32.08% | 39.62% | 20.75% | 13.21% | 3.77% |
| IGFBP2 | Dkk3 | Mac2BP | TGFbeta | IL8 | IL13 | EpCAM | 39.62% | 28.30% | 35.85% | 24.53% | 18.87% | 3.77% |
| IGFBP2 | M2PK | Mac2BP | TGFbeta | TIMP1 | IL8 | IL13 | 39.62% | 26.42% | 33.96% | 24.53% | 16.98% | 9.43% |
| IGFBP2 | Dkk3 | M2PK | Mac2BP | TGFbeta | IL8 | IL13 | 39.62% | 26.42% | 37.74% | 24.53% | 18.87% | 11.32% |
| IGFBP2 | M2PK | Mac2BP | TGFbeta | TIMP1 | IL8 | EpCAM | 39.62% | 26.42% | 35.85% | 22.64% | 24.53% | 13.21% |
| IGFBP2 | Dkk3 | M2PK | Mac2BP | IL8 | IL13 | EpCAM | 39.62% | 26.42% | 37.74% | 18.87% | 16.98% | 9.43% |
| IGFBP2 | Dkk3 | M2PK | Mac2BP | TIMP1 | IL8 | EpCAM | 39.62% | 22.64% | 37.74% | 18.87% | 22.64% | 13.21% |
| IGFBP2 | Dkk3 | Mac2BP | TGFbeta | TIMP1 | IL13 | EpCAM | 39.62% | 22.64% | 37.74% | 15.09% | 16.98% | 7.55% |
| IGFBP2 | Dkk3 | M2PK | Mac2BP | TGFbeta | TIMP1 | IL13 | 37.74% | 26.42% | 32.08% | 22.64% | 20.75% | 7.55% |
| IGFBP2 | M2PK | Mac2BP | TGFbeta | TIMP1 | IL13 | EpCAM | 37.74% | 24.53% | 33.96% | 22.64% | 20.75% | 9.43% |
| IGFBP2 | Dkk3 | M2PK | Mac2BP | TGFbeta | TIMP1 | EpCAM | 37.74% | 24.53% | 35.85% | 20.75% | 22.64% | 11.32% |
| IGFBP2 | Dkk3 | M2PK | Mac2BP | TGFbeta | IL8 | EpCAM | 37.74% | 22.64% | 33.96% | 22.64% | 22.64% | 9.43% |
| IGFBP2 | Dkk3 | M2PK | TGFbeta | TIMP1 | IL8 | IL13 | 37.74% | 22.64% | 28.30% | 20.75% | 9.43% | 3.77% |
| IGFBP2 | Dkk3 | M2PK | TIMP1 | IL8 | IL13 | EpCAM | 37.74% | 22.64% | 30.19% | 18.87% | 9.43% | 5.66% |
| IGFBP2 | Dkk3 | M2PK | TGFbeta | TIMP1 | IL13 | EpCAM | 37.74% | 22.64% | 28.30% | 16.98% | 11.32% | 5.66% |
| IGFBP2 | Dkk3 | M2PK | Mac2BP | TGFbeta | IL13 | EpCAM | 37.74% | 22.64% | 32.08% | 16.98% | 20.75% | 7.55% |
| IGFBP2 | M2PK | TGFbeta | TIMP1 | IL8 | IL13 | EpCAM | 35.85% | 24.53% | 28.30% | 22.64% | 9.43% | 5.66% |
| IGFBP2 | Dkk3 | Mac2BP | TGFbeta | TIMP1 | IL8 | EpCAM | 35.85% | 24.53% | 30.19% | 22.64% | 24.53% | 9.43% |
| IGFBP2 | Dkk3 | TGFbeta | TIMP1 | IL8 | IL13 | EpCAM | 35.85% | 22.64% | 35.85% | 16.98% | 11.32% | 3.77% |
| IGFBP2 | Dkk3 | M2PK | TGFbeta | IL8 | IL13 | EpCAM | 33.96% | 24.53% | 26.42% | 18.87% | 9.43% | 1.89% |
| IGFBP2 | Dkk3 | M2PK | TGFbeta | TIMP1 | IL8 | EpCAM | 33.96% | 20.75% | 33.96% | 15.09% | 9.43% | 7.55% |
| Dkk3 | M2PK | TGFbeta | TIMP1 | IL8 | IL13 | EpCAM | 30.19% | 16.98% | 18.87% | 7.55% | 13.21% | 5.66% |

TABLE 26(b)
Seven biomarker, ten-fold cross validated combination plus gender having >30% sensitivity at 86.4% specificity.

| | | | | | | | Sensitivity | Sensitivity | Sensitivity |
|---|---|---|---|---|---|---|---|---|---|
| | | | | | | | at 86.4% | at 90% | at 95% |
| | | | | | | | specificity | specificity | specificity |
| | | | | | | | cross- | (cross- | (cross- |
| Biomarkers | | | | | | | validated | validated) | validated) |
| IGFBP2 | Mac2BP | DKK3 | IL-8 | EpCAM | TIMP1 | IL-13 | 32.08 | 20.7 | 3.8 |

TABLE 27
Eight biomarker combinations plus gender having >30% sensitivity at
86.4% specificity. No eight marker combinations showed a ten-fold
cross validated sensitivity of >30% at 86.4%.

| | | | | | | | | Sensitivity at | | Sensitivity at 90% | | Sensitivity at 95% | |
|---|---|---|---|---|---|---|---|---|---|---|---|---|---|
| | | | | | | | | 86.4% Specificity | | Specificity | | Specificity | |
| Biomarkers | | | | | | | | Non XV | XV | Non XV | XV | Non XV | XV |
| IGFBP2 | M2PK | Mac2BP | TGFbeta | TIMP1 | IL8 | IL13 | EpCAM | 43.40% | 26.42% | 33.96% | 22.64% | 16.98% | 9.43% |
| IGFBP2 | Dkk3 | Mac2BP | TGFbeta | TIMP1 | IL8 | IL13 | EpCAM | 41.51% | 28.30% | 39.62% | 16.98% | 13.21% | 3.77% |
| IGFBP2 | Dkk3 | M2PK | Mac2BP | TIMP1 | IL8 | IL13 | EpCAM | 41.51% | 26.42% | 32.08% | 18.87% | 15.09% | 9.43% |
| IGFBP2 | Dkk3 | M2PK | Mac2BP | TGFbeta | IL8 | IL13 | EpCAM | 39.62% | 26.42% | 37.74% | 20.75% | 18.87% | 11.32% |
| IGFBP2 | Dkk3 | M2PK | Mac2BP | TGFbeta | TIMP1 | IL8 | IL13 | 39.62% | 24.53% | 32.08% | 24.53% | 16.98% | 5.66% |
| IGFBP2 | Dkk3 | M2PK | Mac2BP | TGFbeta | TIMP1 | IL13 | EpCAM | 39.62% | 22.64% | 32.08% | 20.75% | 18.87% | 7.55% |
| IGFBP2 | Dkk3 | M2PK | Mac2BP | TGFbeta | TIMP1 | IL8 | EpCAM | 39.62% | 22.64% | 37.74% | 18.87% | 22.64% | 13.21% |
| IGFBP2 | Dkk3 | M2PK | TGFbeta | TIMP1 | IL8 | IL13 | EpCAM | 37.74% | 20.75% | 28.30% | 16.98% | 9.43% | 3.77% |
| Dkk3 | M2PK | Mac2BP | TGFbeta | TIMP1 | IL8 | IL13 | EpCAM | 30.19% | 13.21% | 18.87% | 7.55% | 13.21% | 3.77% |

TABLE 28
Nine biomarker combination plus gender having >30% sensitivity at 86.4% specificity.
This combination did not show a ten-fold cross validated sensitivity of >30% at 86.4%.

| | | | | | | | | | Sensitivity at | | Sensitivity at | | Sensitivity at | |
|---|---|---|---|---|---|---|---|---|---|---|---|---|---|---|
| | | | | | | | | | 86.4% Specificity | | 90% Specificity | | 95% Specificity | |
| Biomarkers | | | | | | | | | Non XV | XV | Non XV | XV | Non XV | XV |
| IGFBP2 | Dkk3 | M2PK | Mac2BP | TGFbeta | TIMP1 | IL8 | IL13 | EpCAM | 39.62% | 22.64% | 33.96% | 18.87% | 16.98% | 7.55% |

From the data presented in Tables 13 to 28 it will be apparent that inclusion of demographic terms in the algorithm optimisation process can alter both the number of biomarker combinations showing a sensitivity for APA >30% at 86.4% specificity and/or the maximum sensitivity for detection of advanced adenoma relative to models based on biomarker serum concentrations alone. They also show that the biomarker combinations in the top performing models that include demographic terms differ from those in the top-performing models determined for biomarkers alone. These findings are summarised in Table 29.

TABLE 29
Impact of including demographic terms in the algorithm on the number and maximum sensitivity
of non-cross validated and cross validated Models showing a Sensitivity for APA of >30% at 86.4%
specificity. Non XV, non-ten-fold cross validated models; XV, ten-fold cross validated models. Cells display
number of models with Sensitivity for APA of >30% at 86.4% specificity and the highest Sensitivity achieved
in parenthesis for panels comprising 1-9 biomarkers.

| Biomarker | Biomarkers only | | Biomarkers + Age | | Biomarkers + Gender | |
|---|---|---|---|---|---|---|
| Number | Non XV | XV | Non XV | XV | Non XV | XV |
| 1 | — | — | 1 (30.19%) | 1 (30.19%) | — | — |
| 2 | — | — | 6 (33.96%) | 4 (32.08%) | 7 (41.5%) | 2 (32.08%) |
| 3 | 2 (37.4%) | 1 (30.19) | 24 (35.85%) | 5 (32.08%) | 25 (43.9%) | 6 (33.96%) |
| 4 | 33 (41.51%) | 3 (32.08%) | 49 (39.62%) | 6 (32.08%) | 54 (45.28%) | 16 (33.96%) |
| 5 | 52 (41.51%) | 7 (33.96%) | 63 (39.62%) | 7 (32.08%) | 70 (45.28%) | 18 (33.96%) |
| 6 | 53 (41.51%) | 3 (32.08%) | 56 (39.62%) | 2 (30.19%) | 57 (45.28%) | 7 (32.08%) |
| 7 | 27 (41.45%) | — | 26 (37.74%) | — | 29 (43.4%) | 1 (32.08%) |
| 8 | 8 (37.73%) | — | 8 (37.4%) | — | 9 (43.4%) | — |
| 9 | 1 (35.85%) | — | 1 (33.96%) | — | 1 (39.62%) | — |

Importantly, the number of biomarkers needed in a panel to produce models showing sensitivity for APA >30% at 86.4% specificity was lower when demographic terms were included in the algorithm. Further, for panels comprising 3-5 biomarkers, the number of candidate models showing sensitivity superior to that of FIT was larger when demographic terms were included. Where gender was included as the demographic term, the number of models showing superior cross validated sensitivity was considerably higher than for biomarkers alone or biomarkers plus age. Effective models producing high sensitivity with lower numbers of biomarkers are advantageous as a test product can be produced that assesses a smaller number of biomarkers thereby potentially reducing the cost of goods for the product. Higher numbers of models producing high cross validated sensitivities are also important as it increases the number of candidate models with a high likelihood of performing strongly when applied in a clinical setting.

### Significance of the Results

Greater than 90% of colorectal cancers have their origins in adenomas. For these reasons, clinical guidelines for the management and prevention of colorectal cancer recommend that the colonoscopist remove all polyps and adenomas 5 mm or greater in diameter to reduce the risk of future cancer occurring. Therefore, for colorectal cancer screening applications, while early detection of colorectal cancer remains key, there is increasing focus on the screening tests' abilities to detect APA also.

APAs are typically difficult to detect other than by colonoscopy. The lead, non-colonoscopic colorectal cancer screening test is the fecal immunoassay test (FIT) which detects blood in the stool. As adenoma's bleed less and less often than cancers, there has been increasing focus on developing FITS that are both quantitative and more sensitive than previous tests. By reducing the cut-off level of haemoglobin in stool to trigger a colonoscopy, the sensitivity for APA can be increased but with an increasing number of false positives resulting in more colonoscopies. Published results have suggested FIT can have a sensitivity for APA of around 21% (23.8%, Imperiale et al N Engl J Med 2014; 370:1287-97; 18.8%. Symonds et al. Clinical and Translational Gastroenterology (2016) 7, e137). There is a need for assays that more reliably detect both colorectal cancer and APAs while retaining a high specificity for cancer.

Other systems have been or are being developed with the colorectal cancer screening market in mind. With the exception of a FIT/DNA test recently developed by Exact Science, early candidate blood tests for colorectal cancer are beginning to emerge. These tests particularly focus on genomic and epigenetic markers assessed in blood plasma samples (often referred to as liquid biopsies). One such test examines DNA methylation in the promoter region of the Septin 9 gene and is currently FDA approved for use in the US as a screening test in subjects who have refused to do colonoscopy. A second examines DNA methylation patterns in the promoters of two genes, BCAT1 and IKZF1. This test is currently used in the US in a CLIA lab setting for the detection of colorectal cancer recurrence after surgical and any adjunct chemo- or radiation-therapy. Both are assessed in circulating cell free DNA isolated from around 4 mL of blood plasma. While both tests detect colorectal cancer, their sensitivity for APA is very low with a published value for the Septin 9 test of 11.2% at 91.5% specificity (Church et al. Prospective evaluation of methylated SEPT9 in plasma for detection of asymptomatic colorectal cancer Gut. (2014); 63:317-25) and for the two-marker test, 9.4% at 92% specificity (Symonds et al A Blood Test for Methylated BCAT1 and IKZF1 vs. a Fecal Immunochemical Test for Detection of Colorectal Neoplasia. Clinical and Translational Gastroenterology (2016) 7, e137).

Exact Science's test is a stool test comprising FIT, mutation detection and DNA methylation detection components. This complex and expensive test has been approved for use in screening applications by the FDA in the US. Starting material is a single full stool sample. A sub-sample is removed for use in a FIT and the remainder processed to DNA. Two subsamples of the DNA are used to screen for seven signature point mutations in the K-ras gene and aberrant DNA methylation in the NDRG4 and BMP3 genes. In a trial involving 9989 subjects that yielded 757 APAs, this test differentiated APA form Negative with 42.4% sensitivity at 86.6% specificity.

It is apparent that, of the blood tests considered, the performance of the present blood protein biomarker combinations, inclusive or non-inclusive of age or gender, for the detection of APA is superior to those of the Septin 9 and two gene marker tests. Further as these epigenetic tests require the use of 4 mL of plasma, for diagnostic purposes they will need to be run on a dedicated blood sample. The present blood protein biomarker assays can be run on only a fraction of these volumes meaning that they can be run as one of a battery of serum-based tests on the on serum prepared from a single blood draw.

Of the stool tests considered above, various combinations of the present blood protein biomarker combinations, inclusive or non-inclusive of age or gender, were superior to FIT for the detection of APA. While the performance of Exact Science's FIT/DNA for the detection of APA appears to be superior to that of the present blood protein biomarker combinations, there are other significant advantages to the present test for detection of APA. Firstly, the FIT/DNA test is a stool test with all the compliance disadvantages associated with such tests. Secondly, subjects with clinical conditions that can result in the presence of blood in the stool such as haemorrhoids, colitis, inflammatory bowel diseases and diverticulitis are highly likely to produce false positive results for any test with a FIT component. Even in countries where FITs are offered as National bowel cancer screening programs with testing being available at no charge to the subject, only 40-50% of those invited to participate do so and evidence is accumulating that subjects would much prefer to use a blood test over a stool test (e.g. Adler et al. Improving compliance to colorectal cancer screening using blood and stool based tests in patients refusing screening colonoscopy in Germany. BMC Gastroenterology 2014, 14:183). There is therefore a significant unmet need for an APA screening test that can be used by subjects who can't or won't, for clinical, cultural or personal reasons, use a stool-based test.

Further the present test, based on blood protein biomarkers, plus or minus other demographic variables, is an immunoassay. Such assays are well understood and the systems and infrastructure for running them are widely distributed amongst research, hospital and diagnostic laboratory facilities worldwide. They can also be readily adapted to high throughput diagnostic platforms. Add to this that immunoassays are simple and inexpensive, it is clear that an IVD based on the present, readily accepted, blood protein biomarker technology is better placed to address the mass screening market than the expensive, specialist FIT/DNA test that needs to be run in a specialist central laboratory.

### Example 4 Performance of a 5 Biomarker Panel Including BDNF

To examine the potential utility of a blood-based, five-biomarker panel for the early detection of APA, a case/control study was performed. The 5 protein biomarker combination of tumor M2PK, TIMP-1, IGFBP2, DKK3 and BDNF was evaluated as well as the five biomarkers in combination with additional demographic biomarkers including age, gender and body mass index (BMI).

Such a panel is useful in a number of contexts: As an adjunct to current fecal immunochemical test (FIT) or colonoscopy screening, providing an alternative test for people who cannot or will not test for colorectal neoplasia (cancer or APA) using a stool test; as an additional test to facilitate triage of persons with a positive FIT result for colonoscopy or potentially as an alternative to FIT for first-line colorectal neoplasia screening applications.

The 5 protein biomarkers (M2PK tumor form, TIMP1 IGFBP2, DKK3 and BDNF) were quantified in serum samples from persons diagnosed by colonoscopy as having advanced adenoma and from healthy controls. These values were combined via an algorithm to deliver an APA likelihood score. Optionally additional terms representing values for age, gender and BMI were also included. When used clinically, persons with an APA likelihood score above a defined threshold would be advised by their healthcare professional to progress to colonoscopy for a definitive diagnosis.

To assess the highest sensitivity and specificity with which each protein biomarker individually was able to differentiate between serum samples derived from APA cases and healthy controls, logistic regression analysis was applied to the concentration values determined for each participant sample for each biomarker separately. ROC curve analysis plotting sensitivity against 1-specificity was then used to estimate the point on the ROC curve representing the shortest distance between the ROC curve and the 0:1 position in the Euclidean space represented by the plot. The sensitivities and specificities represented by these points are indicated in Table 30.

TABLE 30
Maximum sensitivity and specificity achieved with each
protein biomarker individually for
differentiating APA samples from healthy controls.

| Biomarker | Sensitivity (%) | Specificity (%) |
|---|---|---|
| PKM2 (tumour form) | 49 | 51 |
| TIMP1 | 54 | 55 |
| IGFBP2 | 49 | 48 |
| DKK3 | 45 | 44 |
| BDNF | 55 | 55 |

These results suggest that of these 5 biomarkers, none, individually, can differentiate between APA and healthy volunteer-derived serum samples with sufficient sensitivity and specificity to be useful clinically.

To determine whether these five biomarkers in combination, optionally coupled with terms for age, gender and BMI, could usefully differentiate between serum samples from APA patients and healthy controls, Logistic regression and ROC curve analysis were again applied.

High performing algorithms, combining concentration values from all 5 protein biomarkers, that differentiated APA cases from controls with highest sensitivity and specificity were trained on the full data set for all cases and controls. For training, 1000 iterations of the logistic regression/ROC analysis process were performed on the shuffled, full data set. The average sensitivities determined at a range of standard specificities are shown in the “Training” column of Table 31.

Lead algorithms identified on training were then tested in-sample using train/test split cross validation. Here the data were train-test split using split ratios of 60:40, 70:30 on shuffled data, with 100 resamples and 1000 iterations to identify the best performing algorithms combining the five biomarker panel set. The Wilson score interval with 95% confidence was calculated manually for top performing algorithm sensitivities with the number of true positives (sensitivity) represented as a binomial distribution ((E. B. Wilson, “Probable inference, the law of succession, and statistical inference,” Journal of the American Statistical Association, vol. 22, no 30 158, pp. 209-212, 1927). The average sensitivity for the best performing cross-validated algorithm is shown in the “Cross-validation” column of Table 31

TABLE 31
Train and test performance parameters for the top-performing
5 protein biomarker algorithm for
the differentiation between APA cases and healthy controls.

| | | Cross-validation** |
|---|---|---|
| | | (In-sample, 70:30 split) |
| Algorithm #1 [100:100 Split] | Training* | average |
| # | 100 | 100 |
| Area under the ROC curve | 66 | 72 |
| Sensitivity (%) [95% CI] | 62 [50-73] | 65 [59-71] |
| Specificity | 73 | 73 |
| Sensitivity (%) at 86% | 60 [47-72] | 63 [51-74] |
| Specificity [95% CI] | | |
| Sensitivity (%) at 90% | 49 [37-61] | 59 [47-70] |
| Specificity [95% CI] | | |
| Sensitivity (%) at 95% | 41 [30-53] | 40 [29-52] |
| Specificity [95% CI] | | |
| Positive Predictive Value (%) | 79.63 | 81.74 |
| Negative Predictive Value (%) | 53.01 | 59.65 |

*In the training column, the first values are discrete (i.e. the performance on the complete training set). The bracketed values are the Wilson Score confidence intervals.
**In the cross validation column, the first values are averaged across 10 different 70:30 data splits. The bracketed values are also the average.

As expected, the sensitivity for APA detection decreased as the specificity increased. Importantly, there was a high level of reproducibility between the sensitivity values determined at the different pre-set specificity values between “training” and “cross-validation” analyses. This indicates that the chosen algorithms are quite robust, suggesting that their accuracy for detecting APA is likely to be acceptably reproducible when applied to fully independent sample sets.

Comparison to the train and test performance parameters for a 4 protein biomarker (IGFBP2, DKK3, TIMP1 and M2PK) (Table 32) demonstrates that there is an improvement in the average test performance parameters (e.g. average sensitivity (%) at 86% specificity and 90% specificity).

TABLE 32
Train and test performance parameters for a 4 protein biomarker
(IGFBP2, DKK3, TIMP1 and M2PK) algorithm for the
differentiation between APA cases and healthy controls.
Train and test performance parameters for the top-performing
5 protein biomarker algorithm for the differentiation
between APA cases and healthy controls.

| | | Cross-validation** |
|---|---|---|
| | | (In-sample, 70:30 split) |
| Algorithm #1 [100:100 Split] | Training* | Average |
| # | 100 | 100 |
| Area under the ROC curve | 67 | 76 |
| Sensitivity (%) [95% CI] | 67 [55-77] | 73 [52-86] |
| Specificity | 68 | 73 |
| Sensitivity (%) at 86% | 57 [45-68] | 61 [41-78] |
| Specificity [95% CI] | | |
| Sensitivity (%) at 90% | 48 [36-60] | 51 [33-70] |
| Specificity [95% CI] | | |
| Sensitivity (%) at 95% | 38 [27-50] | 56 [37-72] |
| Specificity [95% CI] | | |
| Positive Predictive Value (%) | 78.09 | 71.73 |
| Negative Predictive Value (%) | 54.75 | 73.30 |

*In the training column, the first values are discrete (i.e. the performance on the complete training set). The bracketed values are the Wilson Score confidence intervals.
**In the cross validation column, the first values are averaged across 10 different 70:30 data splits. The bracketed values are also the average (e.g., for the 56 [41-69], 41 was the average lower confidence interval across all 10 splits).

Using logistic regression and ROC analysis in a fashion analogous to that described above, the performance of algorithms combining the 5 protein biomarkers, with or without additional demographic terms including age, gender and BMI was also analysed. Age was represented in years and BMI by the calculated index value for the relevant participant. Females were assigned an arbitrary value of 1.1 and males, a value 1.0. Results comparing the in-sample cross validated performance of top performing algorithms for combinations of 5 protein biomarkers only, these 5 biomarkers plus age, 5 biomarkers plus gender, 5 biomarkers plus BMI and 5 biomarkers plus age plus gender plus BMI are shown in Table 33.

TABLE 33
Cross validated performance of top performing algorithms for panels comprising 5 protein biomarkers
(TIMP1, DKK3, M2PK, IGFBP2 and BDNF) alone and in combination with demographic biomarkers age,
gender and BMI. The split ratio used for cross-validation is indicated in parenthesis in the column label.

| | 5 Protein | 5 Biomarkers + | 5 Biomarkers + | 5 Biomarkers + | 5 Biomarkers + | 5 Biomarkers + |
|---|---|---|---|---|---|---|
| Performance | Biomarkers | Age | Gender | BMI | Age + Gender | Age + Gender + BMI |
| Parameter | (70:30) | (60:40) | (70:30) | (60:40) | (60:40) | (60:40) |
| N | 100 | 100 | 100 | 100 | 100 | 100 |
| Area under the | 72 | 74 | 71 | 70 | 73 | 69 |
| ROC curve | | | | | | |
| Sensitivity (%) | 65 [59-71] | 78 [66-86] | 71 [59-81] | 73 [61-82] | 70 [58-80] | 63 [51-74] |
| [95% CI] | | | | | | |
| Euclidian point | | | | | | |
| Specificity % | 73 | 68 | 70 | 65 | 78 | 78 |
| (Euclidian point) | | | | | | |
| Sensitivity (%) at | 63 [51-74] | 48 [36-60] | 52 [40-64] | 56 [44-68] | 60 [48-71] | 54 [42-66] |
| 86% Specificity | | | | | | |
| [95% CI] | | | | | | |
| Sensitivity (%) at | 59 [47-70] | 44 [32-56] | 48 [36-60] | 54 [42-66] | 46 [34-58] | 44 [32-56] |
| 90% Specificity | | | | | | |
| [95% CI] | | | | | | |
| Sensitivity (%) at | 40 [29-52] | 44 [32-56] | 33 [23-45] | 52 [40-64] | 43 [31-55] | 35 [24-47] |
| 95% Specificity | | | | | | |
| [95% CI] | | | | | | |
| Positive Predictive | 81.74 | 80.58 | 80.12 | 78.03 | 84.42 | 82.98 |
| Value (%) | | | | | | |
| Negative Predictive | 59.65 | 64.48 | 58.64 | 58.57 | 60.43 | 55.32 |
| Value (%) | | | | | | |

Algorithms containing all biomarker combinations showed clinically useful differentiation between APA and heathy control samples. While this comparison did not show any significant improvement in the sensitivity for detection of APA in top performing algorithms that include demographic terms, it is possible that the impact of age and gender on the sensitivity of APA detection in this study may have been underestimated as the APA and healthy control serum donors recruited were age and gender matched. Further, the results do not rule out the possibility that inclusion of demographic terms might significantly improve algorithm performance when applied to larger cohorts, cohorts that have not been age and gender matched or in a clinical setting. They do suggest, however, that the levels of the five protein biomarkers are the major contributors to the accuracy with which the top algorithms differentiate between sera derived from patients with APA and healthy controls and that the magnitude of the contribution of any included demographic terms is likely to be lower than that of the five protein biomarkers.

Overall, these results indicate that, when considered in combination, the five biomarker panel can provide a valuable predictor of APA status when compared to other commonly used colorectal neoplasia screening tests. Importantly it seems to outperform FIT with reported performances of FIT for detection of APA varying from 23.8% sensitivity at 94.9% specificity to 49.5% sensitivity at 62.7% specificity (Daly J M et al. Which Fecal Immunochemical Test Should I Choose? Journal of Primary Care & Community Health 2017, Vol. 8 (4) 264-277).

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

APPENDIX 1
Biomarker Sequences

IGFBP2
MLPRVGCPALPLPPPPLLPLLLLLLGASGGGGGARAEVLFRCPPCTPERLAACGPPPVAPPAA
VAAVAGGARMPCAELVREPGCGCCSVCARLEGEACGVYTPRCGQGLRCYPHPGSELPLQAL
VMGEGTCEKRRDAEYGASPEQVADNGDDHSEGGLVENHVDSTMNMLGGGGSAGRKPLKS
GMKELAVFREKVTEQHRQMGKGGKHHLGLEEPKKLRPPPARTPCQQELDQVLERISTMRLPD
ERGPLEHLYSLHIPNCDKHGLYNLKQCKMSLNGQRGECWCVNPNTGKLIQGAPTIRGDPECH
LFYNEQQEARGVHTQRMQ (SEQ ID NO: 1)
DKK3
MQRLGATLLCLLLAAAVPTAPAPAPTATSAPVKPGPALSYPQEEATLNEMFREVEELMEDTQH
KLRSAVEEMEAEEAAAKASSEVNLANLPPSYHNETNTDTKVGNNTIHVHREIHKITNNQTGQM
VFSETVITSVGDEEGRRSHECIIDEDCGPSMYCQFASFQYTCQPCRGQRMLCTRDSECCGDQ
LCVWGHCTKMATRGSNGTICDNQRDCQPGLCCAFQRGLLFPVCTPLPVEGELCHDPASRLL
DLITWELEPDGALDRCPCASGLLCQPHSHSLVYVCKPTFVGSRDQDGEILLPREVPDEYEV
GSFMEEVRQELEDLERSLTEEMALREPAAAAAALLGGEEI (SEQ ID NO: 2)
M2PK (PKM2)
MSKPHSEAGTAFIQTQQLHAAMADTFLEHMCRLDIDSPPITARNTGIICTIGPASRSVETLKEMI
KSGMNVARLNFSHGTHEYHAETIKNVRTATESFASDPILYRPVAVALDTKGPEIRTGLIKGSGT
AEVELKKGATLKITLDNAYMEKCDENILWLDYKNICKVVEVGSKIYVDDGLISLQVKQKGADFLV
TEVENGGSLGSKKGVNLPGAAVDLPAVSEKDIQDLKFGVEQDVDMVFASFIRKASDVHEVRKV
LGEKGKNIKIISKIENHEGVRRFDEILEASDGIMVARGDLGIEIPAEKVFLAQKMMIGRCNRAGKP
VICATQMLESMIKKPRPTRAEGSDVANAVLDGADCIMLSGETAKGDYPLEAVRMQHLIAREAE
AAIYHLQLFEELRRLAPITSDPTEATAVGAVEASFKCCSGAIIVLTKSGRSAHQVARYRPRAPIIA
VTRNPQTARQAHLYRGIFPVLCKDPVQEAWAEDVDLRVNFAMNVGKARGFFKKGDVVIVLTG
WRPGSGFTNTMRVVPVP (SEQ ID NO: 3)
TGFβ
MPPSGLRLLLLLLPLLWLLVLTPGRPAAGLSTCKTIDMELVKRKRIEAIRGQILSKLRLASPPSQG
EVPPGPLPEAVLALYNSTRDRVAGESAEPEPEPEADYYAKEVTRVLMVETHNEIYDKFKQSTH
SIYMFFNTSELREAVPEPVLLSRAELRLLRLKLKVEQHVELYQKYSNNSWRYLSNRLLAPSDSP
EWLSFDVTGVVRQWLSRGGEIEGFRLSAHCSCDSRDNTLQVDINGFTTGRRGDLATIHGMNR
PFLLLMATPLERAQHLQSSRHRRALDTNYCFSSTEKNCCVRQLYIDFRKDLGWKWIHEPKGY
HANFCLGPCPYIWSLDTQYSKVLALYNQHNPGASAAPCCVPQALEPLPIVYYVGRKPKVEQLS
NMIVRSCKCS (SEQ ID NO: 4)
TIMP
MAPFEPLASGLLLLWLIAPSRACTCVPPHPQTAFCNSDLVIRAKFVGTPEVNQTTLYQRYEIKM
TKMYKGFQALGDAADIRFVYTPAMESVCGYFHRSHNRSEEFLIAGKLQDGLLHITTCSFVAPW
NSLSLAQRRGFTKTYTVGCEECTVFPCLSIPCKLQSGTHCLWTDQLLQGSEKGFQSRHLACL
REPGLCTWQSLRSQIA (SEQ ID NO: 5)
IL-8
MTSKLAVALLAAFLISAALCEGAVLPRSAKELRCQCIKTYSKPFHPKFIKELRVIESGPHCANTEII
VKLSDGRELCLDPKENWVQRVVEKFLKRAENS (SEQ ID NO: 6)
IL-13
MHPLLNPLLLALGLMALLLTTVIALTCLGGFASPGPVPPSTALRELIEELVNITQNQKAPLQNGS
MVWSINLTAGMYCAALESLINVSGCSAIEKTQRMLSGFCPHKVSAGQFSSLHVRDTKIEVAQF
VKDLLLHLKKLFREGREN (SEQ ID NO: 7)
Mac2BP
MTPPRLFWVWLLVAGTQGVNDGDMRLADGGATNQGRVEIFYRGQWGTVCDLWDLTDASVV
CRALGFENATQALGRAAFGQGSGPIMLDEVQCTGTEASLADCKSLGWLKSNCRHERDAGVV
CTNETRSTHTLDLSRELSEALGQIFDSQRGCDLSISVNVQGEDALGFCGHTVILTANLEAQALW
KEPGSNVTMSVDAECVPMVRDLLRYFYSRRIDITLSSVKCFHKLASAYGARQLQGYCASLFAIL
LPQDPSFQMPLDLYAYAVATGDALLEKLCLQFLAWNFEALTQAEAWPSVPTDLLQLLLPRSDL
AVPSELALLKAVDTWSWGERASHEEVEGLVEKIRFPMMLPEELFELQFNLSLYWSHEALFQKK
TLQALEFHTVPFQLLARYKGLNLTEDTYKPRIYTSPTWSAFVTDSSWSARKSQLVYQSRRGPL
VKYSSDYFQAPSDYRYYPYQSFQTPQHPSFLFQDKRVSWSLVYLPTIQSCWNYGFSCSSDEL
PVLGLTKSGGSDRTIAYENKALMLCEGLFVADVTDFEGWKAAIPSALDTNSSKSTSSFPCPAG
HFNGFRTVIRPFYLTNSSGVD (SEQ ID NO: 8)
EPCAM
MAPPQVLAFGLLLAAATATFAAAQEECVCENYKLAVNCFVNNNRQCQCTSVGAQNTVICSKLA
AKCLVMKAEMNGSKLGRRAKPEGALQNNDGLYDPDCDESGLFKAKQCNGTSMCWCVNTAG
VRRTDKDTEITCSERVRTYWIIIELKHKAREKPYDSKSLRTALQKEITTRYQLDPKFITSILYENNV
ITIDLVQNSSQKTQNDVDIADVAYYFEKDVKGESLFHSKKMDLTVNGEQLDLDPGQTLIYYVDE
KAPEFSMQGLKAGVIAVIVVVVIAVVAGIVVLVISRKKRMAKYEKAEIKEMGEMHRELNA (SEQ
ID NO: 9)
BDNF
MTILFLTMVISYFGCMKAAPMKEANIRGQGGLAYPGVRTHGTLESVNGPKAGSRGLTSLADTF
EHVIEELLDEDQKVRPNEENNKDADLYTSRVMLSSQVPLEPPLLFLLEEYKNYLDAANMSMRV
RRHSDPARRGELSVCDSISEWVTAADKKTAVDMSGGTVTVLEKVPVSKGQLKQYFYETKCNP
MGYTKEGCRGIDKRHWNSQCRTTQSYVRALTMDSKKRIGWRFIRIDTSCVCTLTIKRGR (SEQ
ID NO: 10)

1. A method for the detection of colorectal pre-cancerous adenomas (APA) in a subject, the method comprising:
determining a measurement for a panel of biomarkers in a biological sample obtained from the subject, the panel comprising
at least IGFBP2 and one or more further biomarkers selected from the group consisting of DKK-3, tumour M2PK, Mac2BP, TGFβ1, TIMP1, IL-8, IL-13 and EpCAM; and
optionally BDNF
wherein the measurement comprises measuring a level of each of the biomarkers in the panel.

2. The method according to claim 1, the panel comprising at least IGFBP2 and one or more further biomarkers selected from the group consisting of DKK-3, tumour M2PK, Mac2BP, TGFβ1, TIMP1, IL-8, IL-13 and EpCAM.

3. The method according to claim 1 or 2, comprising detecting IGFBP2 and two, three, four, five, six or seven further biomarkers selected from the group consisting of DKK-3, tumour M2PK, Mac2BP, TGFβ1, TIMP1, IL-8, IL-13 and EpCAM.

4. The method according to any one of claims 1 to 3, wherein the biomarker panels are selected from:
(i) IGFBP2, Mac2BP, TIMP1; and
(ii) IGFBP2, Mac2BP, TGFβ1.

5. The method according to any one of claims 1 to 4, wherein the biomarker panels are selected from:
(i) IGFBP2, Mac2BP, TGFβ1, IL-13;
(ii) IGFBP2, Mac2BP, TIMP1, IL-13; and
(iii) IGFBP2, Mac2BP, TIMP1, EpCAM.

6. The method according to any one of claims 1 to 5, wherein the biomarker panels are selected from:
(i) IGFBP2, Mac2BP, TGFβ1, TIMP1, EpCAM;
(ii) IGFBP2, M2PK, IL-13, TIMP1, EpCAM;
(iii) IGFBP2, Mac2BP, TGFβ1, M2PK, EpCAM;
(iv) IGFBP2, Mac2BP, IL-13, TIMP1, EpCAM;
(v) IGFBP2, Mac2BP, TGFβ1, TIMP1, IL-13;
(vi) IGFBP2, M2PK, IL-13, TIMP1, IL-8; and
(vii) IGFBP2, Mac2BP, IL-13, TIMP1, DKK3.

7. The method according to any one of claims 1 to 6, wherein the biomarker panel comprises BDNF.

8. The method according to claim 1, wherein the biomarker panels comprise IGFBP2 and TIMP1 and a further one or more biomarker selected from the group consisting of DKK3, BDNF, M2PK, Mac2BP, IL-13 or EpCAM.

9. The method according to claim 1, wherein the biomarker panels comprise IGFBP2, TIMP1 and DKK3 and a further one or more biomarker selected from the group consisting of M2PK, BDNF, Mac2BP, IL-13 and EpCAM.

10. The method according to claim 1, wherein the biomarker panel comprises or consists of IGFBP2, TIMP1, DKK3, M2PK and BDNF.

11. The method according to any one of claims 1 to 10, wherein the subject's age is included as an additional biomarker.

12. A method for the detection of pre-cancerous colorectal adenomas in a subject, the method comprising:
determining a measurement for a panel of biomarkers in a biological sample obtained from the subject, the panel comprising:
at least IGFBP2 and the subject's age as a biomarker and one or more further biomarkers selected from the group consisting of DKK-3, tumour M2PK, Mac2BP, TGFβ1, TIMP1, IL-8, IL-13 and EpCAM; and
optionally BDNF;
wherein the measurement comprises measuring a level of each of the biomarkers in the panel.

13. The method of claim 12, wherein the panel comprises at least IGFBP2 and the subject's age as a biomarker and one or more further biomarkers selected from the group consisting of DKK-3, tumour M2PK, Mac2BP, TGFβ1, TIMP1, IL-8, IL-13 and EpCAM.

14. The method according to claim 12 or 13 wherein the biomarker panels are selected from:
(i) IGFBP2 and Mac2BP;
(ii) IGFBP2 and TGFβ1;
(iii) IGFBP2 and TIMP1;
(iv) IGFBP2 and EpCAM;
(v) IGFBP2 and DKK-3; and
(vi) IGFBP2 and M2PK.

15. The method according to any one of claims 12 to 14, wherein the biomarker panels are selected from:
(i) IGFBP2, Mac2BP and TIMP1;
(ii) IGFBP2, Mac2BP and TGFβ1;
(iii) IGFBP2, Mac2BP and DKK3;
(iv) IGFBP2, TGFβ1 and TIMP1; and
(v) IGFBP2, TGFβ1 and EpCAM.
(vi) IGFBP2, M2PK and IL13

16. The method according to any one of claims 12 to 15, wherein the biomarker panels are selected from:
(i) IGFBP2, Mac2BP, TGFβ1, DKK3;
(ii) IGFBP2, Mac2BP, TGFβ1, TIMP1;
(iii) IGFBP2, Mac2BP, EpCAM, TIMP1;
(iv) IGFBP2, Mac2BP, IL-13, TIMP1;
(v) IGFBP2, Mac2BP, TGFβ1, IL-13; and
(vi) IGFBP2, EpCAM, TGFβ1, DKK3.
(vii) IGFBP2, M2PK, TGFβ1, IL13

17. The method according to any one of claims 12 to 16, wherein the biomarker panels are selected from:
(i) IGFBP2, Mac2BP, TGFβ1, TIMP1, EpCAM;
(ii) IGFBP2, Mac2BP, TGFβ1, TIMP1, M2PK;
(iii) IGFBP2, Mac2BP, TGFβ1, DKK3, IL-13;
(iv) IGFBP2, Mac2BP, TGFβ1, TIMP1, IL-13; and
(v) IGFBP2, Mac2BP, TGFβ1, TIMP1, DKK3.

18. The method according to any one of claims 12 to 17, wherein the biomarker panel comprises BDNF.

19. The method according to claim 12, wherein the biomarker panel comprises IGFBP2 and TIMP1 and a further one or more biomarkers selected from the group consisting of DKK3, BDNF, M2PK, Mac2BP, IL-13 or EpCAM.

20. The method according to claim 12, wherein the biomarker panel comprises IGFBP2, TIMP1 and DKK3 and a further one or more biomarkers selected from the group consisting of M2PK, BDNF, Mac2BP, IL-13 and EpCAM.

21. The method according to claim 12, wherein the biomarker panel comprises or consists of IGFBP2, TIMP1, DKK3, M2PK and BDNF.

22. A method for the detection of pre-cancerous colorectal adenomas in a subject, the method comprising:
determining a measurement for a panel of biomarkers in a biological sample obtained from the subject, the panel comprising:
at least IGFBP2 and the subject's gender as a biomarker and one or more further biomarkers selected from the group consisting of DKK-3, tumour M2PK, Mac2BP, TGFβ1, TIMP1, IL-8, IL-13 and EpCAM; and
optionally BDNF,
wherein the measurement comprises measuring a level of each of the biomarkers in the panel.

23. The method of claim 22, where in the panel comprises at least IGFBP2 and the subject's age as a biomarker and one or more further biomarkers selected from the group consisting of DKK-3, tumour M2PK, Mac2BP, TGFβ1, TIMP1, IL-8, IL-13 and EpCAM.

24. The method according to claim 22 or 23, wherein the biomarker panels are selected from:
(i) IGFBP2 and TIMP1; and
(ii) IGFBP2 and IL-13.

25. The method according to any one of claims 22 to 24, wherein the biomarker panels are selected from:
(i) IGFBP2, Mac2BP, TIMP1;
(ii) IGFBP2, Mac2BP, IL-13;
(iii) IGFBP2, Mac2BP, TGFβ1;
(iv) IGFBP2, IL-8, IL-13;
(v) IGFBP2, DKK-3, IL-13; and
(vi) IGFBP2, IL-13, EpCAM.
(vii) IGFBP2, M2PK, Mac2BP

26. The method according to any one of claims 22 to 25, wherein the biomarker panels are selected from:
(i) IGFBP2, Mac2BP, IL-8, IL-13;
(ii) IGFBP2, Mac2BP, M2PK, TIMP1;
(iii) IGFBP2, Mac2BP, TGFβ1, EpCAM;
(iv) IGFBP2, M2PK, TIMP1, IL-13;
(v) IGFBP2, Mac2BP, M2PK, IL-13;
(vi) IGFBP2, Mac2BP, TGFβ1, TIMP1;
(vii) IGFBP2, IL-8, IL-13, EpCAM;
(viii) IGFBP2, IL-8, IL-13, TIMP1;
(ix) IGFBP2, IL-8, IL-13, DKK3;
(x) IGFBP2, Mac2BP, IL-13, TIMP1;
(xi) IGFBP2, Mac2BP, TGFβ1, IL-13;
(xii) IGFBP2, Mac2BP, DKK3, TIMP1;
(xiii) IGFBP2, EpCAM, IL-13, TIMP1;
(xiv) IGFBP2, Mac2BP, IL-13, DKK3;
(xv) IGFBP2, EpCAM, IL-13, DKK3; and
(xvi) IGFBP2, TGFβ1, IL-13, IL-8.

27. The method according to any one of claims 22 to 26, wherein the biomarker panels are selected from:
(i) IGFBP2, Mac2BP, IL-8, IL-13, EpCAM;
(ii) IGFBP2, TGFβ1, IL-8, IL-13, TIMP1;
(iii) IGFBP2, M2PK, EpCAM, IL-13, TIMP1;
(iv) IGFBP2, Mac2BP, IL-8, IL-13, DKK3;
(v) IGFBP2, Mac2BP, M2PK, IL-13, TGFβ1;
(vi) IGFBP2, DKK3, IL-8, IL-13, EpCAM;
(vii) IGFBP2, M2PK, IL-8, IL-13, TIMP1;
(viii) IGFBP2, Mac2BP, IL-8, TGFβ1, TIMP1;
(ix) IGFBP2, Mac2BP, M2PK, IL-13, EpCAM;
(x) IGFBP2, M2PK, TGFβ1, IL-13, TIMP1;
(xi) IGFBP2, Mac2BP, DKK3, IL-13, TIMP1;
(xii) IGFBP2, Mac2BP; DKK3, IL-8, TIMP1;
(xiii) IGFBP2, Mac2BP, M2PK, TGFβ1, TIMP1;
(xiv) IGFBP2, EpCAM, IL-8, IL-13, TIMP1;
(xv) IGFBP2, M2PK, IL-8, IL-13, EpCAM;
(xvi) IGFBP2, Mac2BP, M2PK, DKK3, IL-13;
(xvii) IGFBP2, Mac2BP, TIMP1, IL-13, EpCAM; and
(xviii) IGFBP2, DKK3, IL-8, IL-13, TIMP1.

28. The method according to any one of claims 22 to 27, wherein the biomarker panel comprises BDNF.

29. The method according to claim 22, wherein the biomarker panel comprises IGFBP2 and TIMP1 and a further one or more biomarkers selected from the group consisting of DKK3, BDNF, M2PK, Mac2BP, IL-13 or EpCAM.

30. The method according to claim 22, wherein the biomarker panel comprises IGFBP2, TIMP1 and DKK3 and a further one or more biomarkers selected from the group consisting of M2PK, BDNF, Mac2BP, IL-13 and EpCAM.

31. The method according to claim 22, wherein the biomarker panel comprises or consists of IGFBP2, TIMP1, DKK3, M2PK and BDNF.

32. The method according to any preceding claim, wherein determining a measurement comprises detecting biomarkers in the biological sample by contacting the sample with detectable binding agents that specifically bind to the biomarkers.

33. The method according to claim 32, wherein the method comprises detecting specific binding between the specific binding agents and the biomarkers using a detection assay.

34. The method according to claim 32 or 33, wherein the binding agent is an antibody.

35. The method according to any preceding claim, wherein determining a measurement comprises measuring the concentration of biomarker in the biological sample.

36. The method according to any preceding claim, wherein determining a measurement comprises performing a statistical analysis.

37. The method according to any preceding claim, wherein the biomarkers are protein biomarkers.

38. The method according to any preceding claim, wherein the biological sample is whole blood, plasma or serum.

39. A method of identifying a subject with APA, the method comprising:
(i) contacting a biological sample obtained from the subject with compounds that specifically and individually bind to a panel of biomarkers according to any one of claims 4-6, 10, 14-17, 21, 24-27 and 31;
(ii) determining the expression or concentration of each biomarker in the sample to obtain a value for each biomarker;
(iii) inputting the values obtained in step (ii) into a logistic regression algorithm;
(iv) comparing the values obtained in step (iii) to a value obtained from the concentration of the same biomarkers in a corresponding biomarker reference panel of case and control samples; and
(v) obtaining a disease likelihood score.

40. A method of screening a subject to identify whether the subject requires further investigation by diagnostic colonoscopy or sigmoidoscopy, comprising:
(i) performing the method according to claim 39; and
(ii) based on the disease score obtained, providing a recommendation for definitive diagnosis by colonoscopy or sigmoidoscopy.

41. A kit for detecting APA in a subject comprising:
(i) one or more compounds that specifically bind to the biomarkers in a biomarker panel according to any one of claims 4-6, 10, 14-17, 21, 24-27 and 31;
(ii) optionally one or more labelled probes that specifically bind to the biomarkers;
(iii) optionally a detection reagent for detecting binding of the one or more labelled probes and/or the one or more compounds to the biomarkers; and
(iv) optionally instructions for use.

42. A method of treating a subject, the method comprising:
(i) performing the method according to claim 39 to obtain a disease score for the subject's risk of APA;
(ii) administering to the subject one or more of colonoscopy with concomitant polypectomy or referral for surgical polyp removal.

43. A method for detecting the presence and/or level of protein biomarkers in a subject suspected of having APA or a patient having APA, the method comprising:
(a) providing a blood, plasma or serum sample obtained from the subject;
(b) contacting the sample with antibodies that specifically bind to IGFBP2 and one or more protein biomarkers in the sample, wherein the one or more protein biomarkers are selected from the group consisting of DKK-3, tumour M2PK, Mac2BP, TGFβ1, TIMP1, IL-8, IL-13 and EpCAM, and optionally BDNF or wherein the protein biomarkers comprise a panel of biomarkers according to any one of claims 4-6, 10, 14-17, 21, 24-27 and 31; and
(c) detecting antibody binding to the protein biomarkers, thereby detecting the presence and/or level of the biomarkers.

44. A composition when used for identifying a subject at risk of APA, the composition comprising one or more labelled compounds that specifically bind to the biomarkers in a biomarker panel according to any one of claims 4-6, 10, 14-17, 21, 24-27 and 31.

45. The method of claim 39 or 43 or the kit of claim 41 or the composition of claim 44, wherein the protein biomarkers comprise a panel of biomarkers according to any one of claims 4-6, 14-17 and 24-27.


SEQUENCE LISTING (10 sequences)

SEQ ID NO: 1 - protein, 325 aa, Homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 2 - protein, 350 aa, Homo sapiens
MQRLGATLLCLLLAAAVPTAPAPAPTATSAPVKPGPALSYPQEEATLNEMFREVEELMEDTQHKLRSAVEEMEAEEAAAKASSEVNLANLPPSYHNETNTDTKVGNNTIHVHREIHKITNNQTGQMVFSETVITSVGDEEGRRSHECIIDEDCGPSMYCQFASFQYTCQPCRGQRMLCTRDSECCGDQLCVWGHCTKMATRGSNGTICDNQRDCQPGLCCAFQRGLLFPVCTPLPVEGELCHDPASRLLDLITWELEPDGALDRCPCASGLLCQPHSHSLVYVCKPTFVGSRDQDGEILLPREVPDEYEVGSFMEEVRQELEDLERSLTEEMALREPAAAAAALLGGEEI

SEQ ID NO: 3 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 4 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 5 - protein, 205 aa, Homo sapiens
MAPFEPLASGLLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLREPGLCTWQSLRSQIA

SEQ ID NO: 6 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 7 - protein, 146 aa, Homo sapiens [sequence omitted]

SEQ ID NO: 8 - protein, 584 aa, Homo sapiens
MTPPRLFWVWLLVAGTQGVNDGDMRLADGGATNQGRVEIFYRGQWGTVCDLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 9 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 10 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR